SEQUENCE LISTING (27 sequences)

SEQ ID NO: 1 - DNA, 1011 bp, Canis familiaris
  CDS (1)..(1011)
atgagggccctgtgggtgctgggcctctgctgcgtcctgctgaccttcgggtcagtccgagctgacgatgaagtcgatgtggatggtacagtggaagaggatctgggtaaaagtagagaaggctccaggacagatgatgaagtagtgcagagagaggaagaagctattcagttggatggattaaatgcatcccaaataagagaacttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaat

SEQ ID NO: 2 - protein, 337 aa, Canis familiaris
MRALWVLGLCCVLLTFGSVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMN

SEQ ID NO: 3 - DNA, 654 bp, Homo sapiens
  CDS (1)..(654)
atgcctgaggaaacccagacccaagaccaaccgatggaggaggaggaggttgagacgttcgcctttcaggcagaaattgcccagttgatgtcattgatcatcaatactttctactcgaacaaagagatctttctgagagagctcatttcaaattcatcagatgcattggacaaaatccggtatgaaagcttgacagatcccagtaaattagactctgggaaagagctgcatattaaccttataccgaacaaacaagatcgaactctcactattgtggatactggaattggaatgaccaaggctgacttgatcaataaccttggtactatcgccaagtctgggaccaaagcgttcatggaagctttgcaggctggtgcagatatctctatgattggccagttcggtgttggtttttattctgcttatttggttgctgagaaagtaactgtgatcaccaaacataacgatgatgagcagtacgcttgggagtcctcagcagggggatcattcacagtgaggacagacacaggtgaacctatgggtcgtggaacaaaagttatcctacacctgaaagaagaccaaactgagtacttggaggaacgaagaataaaggagattgtgaagaaacattctcagtttattggatatcccatt

SEQ ID NO: 4 - protein, 218 aa, Homo sapiens
MPEETQTQDQPMEEEEVETFAFQAEIAQLMSLIINTFYSNKEIFLRELISNSSDALDKIRYESLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPI

SEQ ID NO: 5 - DNA, 2415 bp, Canis familiaris
  CDS (1)..(2415)
atgagggccctgtgggtgctgggcctctgctgcgtcctgctgaccttcgggtcagtccgagctgacgatgaagtcgatgtggatggtacagtggaagaggatctgggtaaaagtagagaaggctccaggacagatgatgaagtagtgcagagagaggaagaagctattcagttggatggattaaatgcatcccaaataagagaacttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 6 - protein, 804 aa, Canis familiaris
MRALWVLGLCCVLLTFGSVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 7 - DNA, 2259 bp, Homo sapiens
  CDS (61)..(2259)
cagttgcttcagcgtcccggtgtggctgtgccgttggtcctgtgcggtcacttagccaagatgcctgaggaaacccagacccaagaccaaccgatggaggaggaggaggttgagacgttcgcctttcaggcagaaattgcccagttgatgtcattgatcatcaatactttctactcgaacaaagagatctttctgagagagctcatttcaaattcatcagatgcattggacaaaatccggtatgaaagcttgacagatcccagtaaattagactctgggaaagagctgcatattaaccttataccgaacaaacaagatcgaactctcactattgtggatactggaattggaatgaccaaggctgacttgatcaataaccttggtactatcgccaagtctgggaccaaagcgttcatggaagctttgcaggctggtgcagatatctctatgattggccagttcggtgttggtttttattctgcttatttggttgctgagaaagtaactgtgatcaccaaacataacgatgatgagcagtacgcttgggagtcctcagcagggggatcattcacagtgaggacagacacaggtgaacctatgggtcgtggaacaaaagttatcctacacctgaaagaagaccaaactgagtacttggaggaacgaagaataaaggagattgtgaagaaacattctcagtttattggatatcccattactctttttgtggagaaggaacgtgataaagaagtaagcgatgatgaggctgaagaaaaggaagacaaagaagaagaaaaagaaaaagaagagaaagagtcggaagacaaacctgaaattgaagatgttggttctgatgaggaagaagaaaagaaggatggtgacaagaagaagaagaagaagattaaggaaaagtacatcgatcaagaagagctcaacaaaacaaagcccatctggaccagaaatcccgacgatattactaatgaggagtacggagaattctataagagcttgaccaatgactgggaagatcacttggcagtgaagcatttttcagttgaaggacagttggaattcagagcccttctatttgtcccacgacgtgctccttttgatctgtttgaaaacagaaagaaaaagaacaacatcaaattgtatgtacgcagagttttcatcatggataactgtgaggagctaatccctgaatatctgaacttcattagaggggtggtagactcggaggatctccctctaaacatatcccgtgagatgttgcaacaaagcaaaattttgaaagttatcaggaagaatttggtcaaaaaatgcttagaactctttactgaactggcggaagataaagagaactacaagaaattctatgagcagttctctaaaaacataaagcttggaatacacgaagactctcaaaatcggaagaagctttcagagctgttaaggtactacacatctgcctctggtgatgagatggtttctctcaaggactactgcaccagaatgaaggagaaccagaaacatatctattatatcacaggtgagaccaaggaccaggtagctaactcagcctttgtggaacgtcttcggaaacatggcttagaagtgatctatatgattgagcccattgatgagtactgtgtccaacagctgaaggaatttgaggggaagactttagtgtcagtcaccaaagaaggcctggaacttccagaggatgaagaagagaaaaagaagcaggaagagaaaaaaacaaagtttgagaacctctgcaaaatcatgaaagacatattggagaaaaaagttgaaaaggtggttgtgtcaaaccgattggtgacatctccatgctgtattgtcacaagcacatatggctggacagcaaacatggagagaatcatgaaagctcaagccctaagagacaactcaacaatgggttacatggcagcaaagaaacacctggagataaaccctgaccattccattattgagaccttaaggcaaaaggcagaggctgataagaacgacaagtctgtgaaggatctggtcatcttgctttatgaaactgcgctcctgtcttctggcttcagtctggaagatccccagacacatgctaacaggatctacaggatgatcaaacttggtctgggtattgatgaagatgaccctactgctgatgataccagtgctgctgtaactgaagaaatgccaccccttgaaggagatgacgacacatcacgcatggaagaagtagactaa

SEQ ID NO: 8 - protein, 732 aa, Homo sapiens
MPEETQTQDQPMEEEEVETFAFQAEIAQLMSLIINTFYSNKEIFLRELISNSSDALDKIRYESLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD

SEQ ID NO: 9 - DNA, 1725 bp, Homo sapiens
  CDS (63)..(1592)
ggccggtagctgttgctgttgggggaccccctcattcctgccgctgccgtccctgctgcctcatggcggccatcggagttcacctgggctgcacctcagcctgtgtggccgtctataaggatggccgggctggtgtggttgcaaatgatgccggtgaccgagttactccagctgttgttgcttactcagaaaatgaagagattgttggattggcagcaaaacaaagtagaataagaaatatttcaaatacagtaatgaaagtaaagcagatcctgggcagaagctccagtgatccacaagctcagaaatacatcgcggaaagtaaatgtttagtcattgaaaaaaatgggaaattacgatatgaaatagatactggagaagaaacaaaatttgttaacccagaagatgttgccagactgatatttagtaaaatgaaagaaacggcacattctgtattgggctcagatgcaaatgatgtagttattactgtcccgtttgattttggagaaaagcaaaaaaatgctcttggagaagcagctagagctgctggatttaatgttttgcgattaattcacgaaccgtctgcagctcttcttgcttatggaattggacaagactcccctactggaaaaagcaatattttggtgtttaagcttggaggaacatccttatctctcagcgtcatggaagttaacagtggaatatatcgggttctttcaacaaacactgatgataacatcggtggtgcacatttcacagaaaccttagcacagtatctagcttctgagttccaaagatccttcaaacatgatgtgagaggaaatgcgcgagccatgatgaaattaacgaacagtgctgaagtagcgaaacattctttgtcaaccttgggaagtgccaactgttttcttgactcattatatgaaggtcaagattttgattgcaatgtgtccagagcaagatttgaacttctttgttctccactttttaataagtgtatagaagcaatcagaggactcttagatcaaaatggatttacagcagatgatatcaacaaggttgtcctttgtggagggtcttctcgaatcccaaagctacagcaactgattaaagatcttttcccagctgttgagcttctcaattctatccctcctgatgaagtgatccctattggtgcagctatagaagcaggaattcttattgggaaagaaaacctgttggtggaagactctcttatgatagagtgttcagccagagatattttagttaagggtgtggacgaatcaggagccagtagattcacagtgctgtttccatcagggactcctttgccagctcgaagacaacacacattgcaagcccctggaagcatatcttcagtgtgccttgaactctatgagtctgatgggaagaactctgccaaagaggaaaccaagtttgcacaggttgtactccaggatttagataaaaaagaaaatggattacgtgatatattagctgttcttactatgaaaagggatggatctttacatgtgacatgcacagatcaagaaactggaaaatgtgaagcaatctctattgagatagcatcttagtgttttagagaaatcaagaatttttaaaaacaagaatatcaacatttggttttgtgtataagtggtgtttgtattaaaatactttttcaatgaactgtataaactatgttttattaaactacaatatatcagt

SEQ ID NO: 10 - protein, 509 aa, Homo sapiens
MAAIGVHLGCTSACVAVYKDGRAGVVANDAGDRVTPAVVAYSENEEIVGLAAKQSRIRNISNTVMKVKQILGRSSSDPQAQKYIAESKCLVIEKNGKLRYEIDTGEETKFVNPEDVARLIFSKMKETAHSVLGSDANDVVITVPFDFGEKQKNALGEAARAAGFNVLRLIHEPSAALLAYGIGQDSPTGKSNILVFKLGGTSLSLSVMEVNSGIYRVLSTNTDDNIGGAHFTETLAQYLASEFQRSFKHDVRGNARAMMKLTNSAEVAKHSLSTLGSANCFLDSLYEGQDFDCNVSRARFELLCSPLFNKCIEAIRGLLDQNGFTADDINKVVLCGGSSRIPKLQQLIKDLFPAVELLNSIPPDEVIPIGAAIEAGILIGKENLLVEDSLMIECSARDILVKGVDESGASRFTVLFPSGTPLPARRQHTLQAPGSISSVCLELYESDGKNSAKEETKFAQVVLQDLDKKENGLRDILAVLTMKRDGSLHVTCTDQETGKCEAISIEIAS

SEQ ID NO: 11 - DNA, 2202 bp, Homo sapiens
  CDS (25)..(1746)
cacgcttgccgccgccccgcagaaatgcttcggttacccacagtctttcgccagatgagaccggtgtccagggtactggctcctcatctcactcgggcttatgccaaagatgtaaaatttggtgcagatgcccgagccttaatgcttcaaggtgtagaccttttagccgatgctgtggccgttacaatggggccaaagggaagaacagtgattattgagcagggttggggaagtcccaaagtaacaaaagatggtgtgactgttgcaaagtcaattgacttaaaagataaatacaagaacattggagctaaacttgttcaagatgttgccaataacacaaatgaagaagctggggatggcactaccactgctactgtactggcacgctctatagccaaggaaggcttcgagaagattagcaaaggtgctaatccagtggaaatcaggagaggtgtgatgttagctgttgatgctgtaattgctgaacttaaaaagcagtctaaacctgtgaccacccctgaagaaattgcacaggttgctacgatttctgcaaacggagacaaagaaattggcaatatcatctctgatgcaatgaaaaaagttggaagaaagggtgtcatcacagtaaaggatggaaaaacactgaatgatgaattagaaattattgaaggcatgaagtttgatcgaggctatatttctccatactttattaatacatcaaaaggtcagaaatgtgaattccaggatgcctatgttctgttgagtgaaaagaaaatttctagtatccagtccattgtacctgctcttgaaattgccaatgctcaccgtaagcctttggtcataatcgctgaagatgttgatggagaagctctaagtacactcgtcttgaataggctaaaggttggtcttcaggttgtggcagtcaaggctccagggtttggtgacaatagaaagaaccagcttaaagatatggctattgctactggtggtgcagtgtttggagaagagggattgaccctgaatcttgaagacgttcagcctcatgacttaggaaaagttggagaggtcattgtgaccaaagacgatgccatgctcttaaaaggaaaaggtgacaaggctcaaattgaaaaacgtattcaagaaatcattgagcagttagatgtcacaactagtgaatatgaaaaggaaaaactgaatgaacggcttgcaaaactttcagatggagtggctgtgctgaaggttggtgggacaagtgatgttgaagtgaatgaaaagaaagacagagttacagatgcccttaatgctacaagagctgctgttgaagaaggcattgttttgggagggggttgtgccctccttcgatgcattccagccttggactcattgactccagctaatgaagatcaaaaaattggtatagaaattattaaaagaacactcaaaattccagcaatgaccattgctaagaatgcaggtgttgaaggatctttgatagttgagaaaattatgcaaagttcctcagaagttggttatgatgctatggctggagattttgtgaatatggtggaaaaaggaatcattgacccaacaaaggttgtgagaactgctttattggatgctgctggtgtggcctctctgttaactacagcagaagttgtagtcacagaaattcctaaagaagagaaggaccctggaatgggtgcaatgggtggaatgggaggtggtatgggaggtggcatgttctaactcctagactagtgctttacctttattaatgaactgtgacaggaagcccaaggcagtgttcctcaccaataacttcagagaagtcagttggagaaaatgaagaaaaaggctggctgaaaatcactataaccatcagttactggtttcagttgacaaaatatataatggtttactgctgtcattgtccatgcctacagataatttattttgtatttttgaataaaaaacatttgtacattcctgatactgggtacaagagccatgtaccagtgtactgctttcaacttaaatcactgaggcatttttactactattctgttaaaatcaggattttagtgcttgccaccaccagatgagaagttaagcagcctttctgtggagagtgagaataattgtgtacaaagtagagaagtatccaattatgtgacaacctttgtgtaataaaaatttgtttaa

SEQ ID NO: 12 - protein, 573 aa, Homo sapiens
MLRLPTVFRQMRPVSRVLAPHLTRAYAKDVKFGADARALMLQGVDLLADAVAVTMGPKGRTVIIEQGWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 13 - DNA, 1940 bp, Homo sapiens
  CDS (63)..(1316)
gcagagccgctgccggagggtcgttttaaagggcccgcgcgttgccgccccctcggcccgccatgctgctatccgtgccgctgctgctcggcctcctcggcctggccgtcgccgagcctgccgtctacttcaaggagcagtttctggacggagacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctgtagagaggcctgcctccagggctggactgaggcctgagcgctcctgccgcagagcttgccgcgccaaataatgtctctgtgagactcgagaactttcatttttttccaggctggttcggatttggggtggattttggttttgttcccctcctccactctcccccaccccctccccgccctttttttttttttttttaaactggtattttatctttgattctccttcagccctcacccctggttctcatctttcttgatcaacatcttttcttgcctctgtccccttctctcatctcttagctcccctccaacctggggggcagtggtgtggagaagccacaggcctgagatttcatctgctctccttcctggagcccagaggagggcagcagaagggggtggtgtctccaaccccccagcactgaggaagaacggggctcttctcatttcacccctccctttctcccctgcccccaggactgggccacttctgggtggggcagtgggtcccagattggctcacactgagaatgtaagaactacaaacaaaatttctattaaattaaattttgtgtctccaaaaaaaaaaaaaaaaaaaaaaaaaaaaaaccaaaaaaaaaaaaaaaa

SEQ ID NO: 14 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 15 - DNA, 207 bp, Canis familiaris
  CDS (1)..(207)
agtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacg

SEQ ID NO: 16 - protein, 69 aa, Canis familiaris
SEKTKESREAIEKEFEPLLNWMKDKALKDKIEKAVVSQRLTESPCALVASQYGWSGNMERIMKAQAYQT

SEQ ID NO: 17 - DNA, 201 bp, Homo sapiens
  CDS (1)..(201)
gatgaagaagagaaaaagaagcaggaagagaaaaaaacaaagtttgagaacctctgcaaaatcatgaaagacatattggagaaaaaagttgaaaaggtggttgtgtcaaaccgattggtgacatctccatgctgtattgtcacaagcacatatggctggacagcaaacatggagagaatcatgaaagctcaagccctaaga

SEQ ID NO: 18 - protein, 67 aa, Homo sapiens
DEEEKKKQEEKKTKFENLCKIMKDILEKKVEKVVVSNRLVTSPCCIVTSTYGWTANMERIMKAQALR

SEQ ID NO: 19 - DNA, 666 bp, Homo sapiens
  CDS (1)..(666)
gtgctgctccttgatgtcactcccctgtctctgggtattgaaactctaggaggtgtctttaccaaacttattaataggaataccactattccaaccaagaagagccaggtattctctactgccgctgatggtcaaacgcaagtggaaattaaagtgtgtcagggtgaaagagagatggctggagacaacaaactccttggacagtttactttgattggaattccaccagcccctcgtggagttcctcagattgaagttacatttgacattgatgccaatgggatagtacatgtttctgctaaagataaaggcacaggacgtgagcagcagattgtaatccagtcttctggtggattaagcaaagatgatattgaaaatatggttaaaaatgcagagaaatatgctgaagaagaccggcgaaagaaggaacgagttgaagcagttaatatggctgaaggaatcattcacgacacagaaaccaagatggaagaattcaaggaccaattacctgctgatgagtgcaacaagctgaaagaagagatttccaaaatgagggagctcctggctagaaaagacagcgaaacaggagaaaatattagacaggcagcatcctctcttcagcaggcatcactgaagctgttcgaaatggcatacaaaaagatggca

SEQ ID NO: 20 - protein, 222 aa, Homo sapiens
VLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTGREQQIVIQSSGGLSKDDIENMVKNAEKYAEEDRRKKERVEAVNMAEGIIHDTETKMEEFKDQLPADECNKLKEEISKMRELLARKDSETGENIRQAASSLQQASLKLFEMAYKKMA

SEQ ID NO: 21 - DNA, 2400 bp, Canis familiaris
  CDS (1)..(2400)
atgagggccctgtgggtgctgggcctctgctgcgtcctgctgaccttcgggtcagtccgagctgacgatgaagtcgatgtggatggtacagtggaagaggatctgggtaaaagtagagaaggctccaggacagatgatgaagtagtgcagagagaggaagaagctattcagttggatggattaaatgcatcccaaataagagaacttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaa

SEQ ID NO: 22 - protein, 800 aa, Canis familiaris
MRALWVLGLCCVLLTFGSVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAE

SEQ ID NO: 23 - protein, 4 aa, synthetic construct
KDEL

SEQ ID NO: 24 - DNA, 26 bp, Canis familiaris
gcgtcgacagggccctgtgggtgctg

SEQ ID NO: 25 - DNA, 31 bp, Canis familiaris
gcgcggccgctcattcagctgtagatttctt

SEQ ID NO: 26 - DNA, 26 bp, Canis familiaris
gcgtcgacagggccctgtgggtgctg

SEQ ID NO: 27 - DNA, 34 bp, Canis familiaris
gcgcggccgctcaattcataagctcccaatccca

(19) United States
(12) Patent Application Publication
(10) Pub. No.: US 2003/0216315 A1
(43) Pub. Date: Nov. 20, 2003

(54) Modulation of immune response by non-peptide binding stress response polypeptides

(75) Inventors: Christopher V. Nicchitta, Durham, NC (US); Julie C. Baker-LePain, Durham, NC (US)

Correspondence Address: JENKINS & WILSON, PA, 3100 TOWER BLVD, SUITE 1400, DURHAM, NC, 27707, US

(73) Assignee: Duke University

(21) Appl. No.: 10/367,093

(22) Filed: Feb. 13, 2003

Related U.S. Application Data
(60) Provisional application No. 60/356,293, filed on Feb. 13, 2002.

(51) Int. Cl. A61K 38/17; C07K 14/71

(52) U.S. Cl. 514/12; 530/350

(57) ABSTRACT

A recombinant stress response polypeptide that lacks an antigen binding domain, and methods for using the recombinant stress response polypeptide to elicit an immune response, for example an anti-tumor response, in a subject.

### CROSS REFERENCE TO RELATED APPLICATIONS

This application is based on and claims priority to U.S. Provisional Application Serial No. 60/356,293, filed Feb. 13, 2002, herein incorporated by reference in its entirety.

### GRANT STATEMENT

This work was supported by grant number DK53058 from the United States National Institutes of Health. Thus, the U.S. Government has certain rights in the invention.

### TECHNICAL FIELD

The present invention relates to compositions and methods pertaining to the modulation of an immune response by a stress response polypeptide free of an antigen binding domain. In a preferred embodiment, the present invention relates to a recombinant GRP94 polypeptide free of an antigen binding domain, and therapeutic methods associated therewith.

Table of Abbreviations

| | |
|---|---|
| 4T1 | mammary carcinoma cells |
| APCs | antigen presenting cells |
| BSA | bovine serum albumin |
| CD40 | APO co-stimulatory molecule |
| CD80 | APC co-stimulatory molecule |
| CD86 | APC co-stimulatory molecule |
| CD91 | Hsp receptor on APCs |
| CTL | cytotoxic T lymphocyte(s) |
| DCs | dendritic cells |
| DMEM | Dulbecco's modified Eagle's |
| | medium |
| Endo H | endonuclease H |
| ER | endoplasmic reticulum |
| ERD-2 | Event-Related Desynchronization; |
| | an endoplasmic reticulum |
| | retention protein |
| Fc | antibody antigen-binding fragment |
| GRP94 | glucose regulated protein of 94 |
| | kDa, ER paralog of the Hsp90 |
| | family of chaperones |
| GRPΔKDEL or | secreted form of GRP94 |
| GRP94ΔKDEL | |
| Hsp(s) | heat shock protein(s) |
| Hsp70 | any member of the Hsp70 family of |
| | heat shock proteins |
| HSP70 | heat shock protein of 70 kDa |
| Hsp90 | any member of the Hsp90 family of |
| | heat shock protein |
| HSP90 | heat shock protein of 90 kDa |
| IFN | interferon |
| Ig | immunoglobulin |
| IGF-1 | insulin-like growth factor |
| IgG | immunoglobulin G |
| IL | interleukin |
| MHC | major histocompatability complex |
| MLTC | mixed lymphocyte tumor cell assay |
| myc | antigenic peptide tag |
| NIH3T3 | fibroblast cells |
| NK | natural killer cell |
| NTD | NH2-terminal geldanamycin-binding |
| | domain |
| PAGE | polyacrylamide gel electrophoresis |
| PCR | Polymerase Chain Reaction |
| PBS | phosphate buffered saline |
| pEF/my/cyto | vector |
| PNGase-F | peptide N-glycosidase F |
| rpm | revolutions per minute |
| SDS | sodium dodecyl sulfate |
| TNF | tumor necrosis factor |

### BACKGROUND ART

Modulation of immune response has become an important strategy for combating infection and disease. A significant effort in the design of vaccines and therapeutics has focused on identification of antigens selectively present in tumor cells and pathogen infected-cells. The role of stress response polypeptides (also called chaperone proteins and heat shock proteins) in providing tumor immunity has been attributed to their role as chaperone proteins and the antigenicity of peptides bound thereto.

Within cell, stress response proteins are bound to diverse peptide antigens, and thus bear the immunological identity of the cell of origin (Udono & Srivastava, 1993; Blachere & Srivastava, 1995; Nieland et al., 1996; Lammert et al., 1997; Spee & Neefjes, 1997; Breloer et al., 1998). Following their release from cells, chaperone-peptide complexes are internalized by professional antigen presenting cells (APCs) via a receptor-mediated process (Arnold-Schild et al., 1999; Wassenberg et al., 1999; Binder et al., 2000a; Castellino et al., 2000; Singh-Jasuja et al., 2000b; Basu et al., 2001). Subsequent to internalization, bound peptides are transferred to major histocompatability molecules for re-presentation and subsequent T lymphocyte activation (Arnold et al., 1995; Suto & Srivastava, 1995; Arnold et al., 1997; Blachere et al., 1997; Schild et al., 1999).

Despite the importance of antigenic peptides in eliciting an anti-tumor response, the identity of a single or small group of peptides that can confer immunity has remained elusive. Vaccines prepared from cancers, including cancers induced by chemical carcinogens or ultraviolet radiation as well as spontaneous cancers, are immunogenic in syngenic hosts. However, immunity appears to be limited to the cancer of vaccine origin.

A current interpretation of these data reflects the following: (1) the immunogenicity of cancers results not from one or a few cancer-specific peptides but from a large and complex array of them; (2) the continuous cell division and genomic instability of cancer cells facilitates the accumulation of mutated peptides, which become antigenic by virtue of their presentation by MHC alleles; (3) the randomness of genetic mutation leads to an individually specific “antigenic fingerprint” for each cancer; and (4) the mutational repertoire that becomes immunogenic is incidental to the transformation process. See e.g., Basu & Srivastava (2000) Cell Stress Chaperones 5:443-451.

In addition to their function as peptide binding proteins, recent results suggest that stress response proteins can also activate expression of co-stimulatory molecules on dendritic cells, which is required to elicit a CTL response (Chen et al., 1999; Todryk et al., 1999; Asea et al., 2000b; Basu et al., 2000; Binder et al., 2000b; Kol et al., 2000; Ohashi et al., 2000; Singh-Jasuja et al., 2000a). Such activities are not dependent on the identity of bound peptide antigens. Thus, the mechanism of action of chaperone-peptide complexes includes both innate and adaptive immune responses.

Based on the foregoing observations, immunization approaches for eliciting anti-tumor and anti-infective immunity have chaperone-peptide complexes purified from tissue homogenates. Using this strategy, preliminary outcomes in human clinical trials are promising. See Janetzki et al. (2000) Int J Cancer 88:232-238; Amato et al. (1999) ASCO Meeting abstract; Amato et al. (2000) ASCO Meeting abstract; and Eton et al. (2000) Proc Am Assoc Canc Res 41:543.

Still, there exists a long-felt need in the art to develop safe and broadly applicable immunostimulatory therapies. To meet this need, the present invention provides a stress response polypeptide free of an antigen binding domain. As disclosed herein, administration of a stress response polypeptide to a subject, wherein the stress response polypeptide is free of an antigen binding domain, can elicit both non-specific and specific immune responses.

### SUMMARY OF THE INVENTION

The present invention provides a recombinant stress response polypeptide free of an antigen binding domain. When expressed in a host cell, the recombinant stress response polypeptide polypeptide is transported extracellularly. Alternatively, a recombinant stress response polypeptide of the present invention can be provided extracellularly to a cell in need of treatment.

A recombinant stress response polypeptide of the present invention can be prepared based on the sequence of a Hsp 60 polypeptide, a Hsp70 polypeptide, a Hsp9O polypeptide, or a calreticulin polypeptide and can be obtained from any organism. In preferred embodiments of the invention, the recombinant stress response polypeptide comprises a recombinant GRP94 polypeptide or a recombinant HSP90 polypeptide.

A recombinant GRP94 polypeptide of the present invention, wherein the recombinant GRP94 polypeptide lacks an antigen binding site, can comprise: (a)a polypeptide comprising an amino acid sequence of SEQ ID NO:2; (b) a polypeptide substantially identical to SEQ ID NO:2; (c) a polypeptide encoded by a nucleic acid of SEQ ID NO:1; or (d) a polypeptide peptide encoded by a nucleic acid substantially identical to SEQ ID NO:1.

A recombinant GRP94 polypeptide of the present invention can also comprise: (a) an isolated nucleic acid molecule that hybridizes to a nucleic acid comprising a nucleotide sequence of SEQ ID NO:1 under wash stringency conditions represented by a wash solution having less than about 200 mM salt concentration and a wash temperature of greater than about 45° C., and that encodes a GRP94 polypeptide free of an antigen binding domain; and (b) an isolated nucleic acid differing by at least one functionally equivalent codon from the isolated nucleic acid molecule of (a) above in nucleic acid sequence due to the degeneracy of the genetic code, and that encodes a GRP94 polypeptide encoded by the isolated nucleic acid of (a) above.

The present invention further provides a composition for eliciting an immune response in a subject. In a preferred embodiment, the composition comprises: (a) an immunostimulatory amount of a recombinant stress response polypeptide free of an antigen binding domain; and (b) a pharmaceutically acceptable carrier.

Also provided is a method for eliciting an immune response in a subject by administering to a subject a recombinant stress response polypeptide free of an antigenic peptide binding site.

An immune response elicited by a recombinant stress response polypeptide of the present invention can comprise an innate immune response, an adaptive immune response, or a combination thereof. Preferably, an innate immune response comprises dendritic cell maturation, and an adaptive immune response comprises an anti-tumor or anti-infection response.

The present invention further provides a method for inhibiting tumor growth in a subject, the method comprising: (a) transfecting a culture of healthy cells with a construct encoding a stress response polypeptide, wherein the stress response polypeptide comprises an extracellularly transported polypeptide when expressed in the healthy cell; and (b) administering to a subject the culture of transfected healthy cells, whereby tumor growth in the subject is inhibited.

Also provided is a method for inhibiting tumor metastasis in a subject, the method comprising: (a) transfecting a culture of healthy cells with a construct encoding a stress response polypeptide, wherein the stress response polypeptide comprises an extracellularly transported polypeptide when expressed in the healthy cell; and (b) administering to a subject the culture of transfected healthy cells, whereby tumor metastasis in the subject is inhibited.

Thus, the present invention further provides a method for inhibiting tumor growth via administering to a subject a recombinant stress response polypeptide free of an antigen binding site. Also provided is a method for inhibiting tumor metastases via administering to a subject a recombinant stress response polypeptide free of an antigenic peptide binding site.

The compositions and methods of the present invention are suitable for administration to any subject in need of treatment, including mammals and humans.

Accordingly, it is an object of the present invention to provide novel compositions comprising recombinant stress response polypeptides that are useful for eliciting an immune response in a subject. The object is achieved in whole or in part by the present invention.

An object of the invention having been stated hereinabove, other objects will become evident as the description proceeds when taken in connection with the accompanying Drawings and Laboratory Examples as best described herein below.

### BRIEF DESCRIPTION OF THE DRAWINGS

FIGS. 1A-1J show that vaccination with 4T1 mammary carcinoma cells or NIH3T3 fibroblast cells secreting GRPΔKDEL leads to delayed tumor growth rates and decreased tumor metastasis.

FIG. 1A is a picture of a polyacrylamide gel showing that transfected, irradiated cells secrete GRPΔKDEL. 4T1 cells were transfected with GRPΔKDEL (T and T,I) or mock-transfected (Mock). At 24 hours post-transfection, cells were either irradiated with 10,000 rads (T,I) or left non-irradiated (Mock and T). At 72 hours post-transfection, cells were metabolically labeled, and GRP94 was recovered from the media by immunoprecipitation. Immunoprecipitated proteins were resolved by SDS-PAGE.

FIGS. 1B-1I are graphs depicting tumor volume (mm³) or lung weight following vaccination and tumor challenge. Female BALB/c mice were vaccinated weekly for four consecutive weeks by intradermal injection of PBS (negative control), mock-transfected 4T1 cells, GRPΔKDEL-transfected 4T1 cells, mock-transfected NIH3T3 cells, or GRPΔKDEL-transfected NIH3T3 cells. On the fifth week, animals in each group were challenged with 1×10⁶ non-irradiated 4T1 cells by intradermal injection at a remote site. Following sacrifice, lungs were resected from mice in each group and weighed as a measure of tumor metastasis. Tumor volume and lung weight were determined as described in Example 5.

FIG. 1B is a graph depicting tumor volume (mm³) following vaccination with PBS (negative control). Tumor volume was determined at each of the days following post-transfection, as indicated. Each line represents a growth curve for an individual subject.

FIG. 1C is a graph depicting tumor volume (mm³) following vaccination with 2-4×10⁶ mock-transfected 4T1 cells. Tumor volume was determined at each of the days following post-transfection, as indicated. Each line represents a growth curve for an individual subject.

FIG. 1D is a graph depicting tumor volume (mm³) following vaccination with 2-4×10⁶ GRPΔKDEL-transfected 4T1 cells. Tumor volume was determined at each of the days following post-transfection, as indicated. Each line represents a growth curve for an individual subject.

FIG. 1E is a graph depicting average tumor volume (mm³) following vaccination with PBS (PBS, solid line), mock-transfected 4T1 cells (4T1-Mock, dashed line), or GRPΔKDEL-transfected 4T1 cells (4T1-ΔKDEL, dashed line marked with circles (●)). Tumor volume was determined at each of the days following post-transfection, as indicated.

FIG. 1F is a graph depicting tumor volume (mm³) following vaccination with 2-4×10⁶ mock-transfected NIH3T3 cells. Tumor volume was determined at each of the days following post-transfection, as indicated. Each line represents a growth curve for an individual subject.

FIG. 1G is a graph depicting tumor volume (mm³) following vaccination with 2-4×10⁶ GRPΔKDEL-transfected NIH3T3 cells. Tumor volume was determined at each of the days following post-transfection, as indicated. Each line represents a growth curve for an individual subject.

FIG. 1H is a graph depicting average tumor volume (mm³) following vaccination with PBS (PBS, solid line), mock-transfected NIH3T3 cells (NIH-Mock, dashed line), or GRPΔKDEL-transfected NIH3T3 cells (NIH-ΔKDEL, dashed line marked with circles (●)). Tumor volume was determined at each of the days following post-transfection, as indicated.

FIG. 11 is a bar graph depicting average lung weight (g) following vaccination and tumor challenge. Asterisks indicate a significantly lower average lung weight following vaccination with GRPΔKDEL-transfected 4T1 cells or GRPΔKDEL-transfected NIH3T3 cells when compared to controls (p=0.0012 for 4T1-ΔKDEL, p=0.025 for NIH-ΔKDEL by Wilcoxon rank sum test).

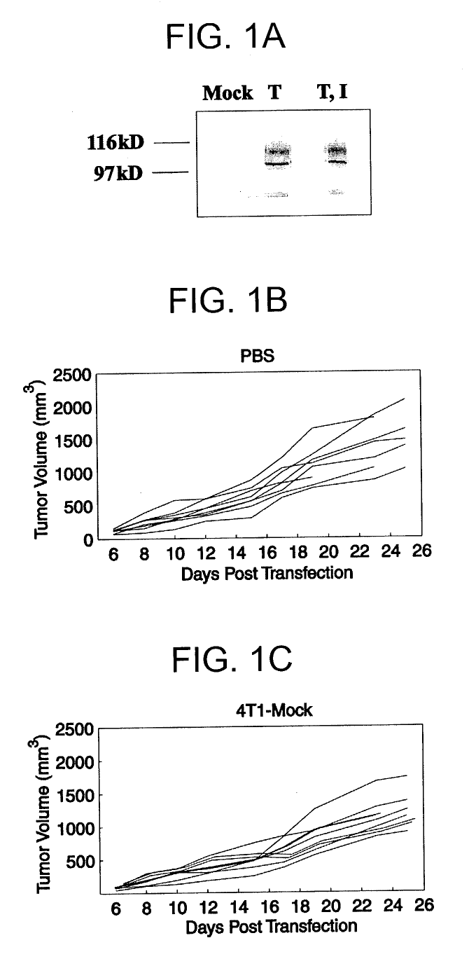

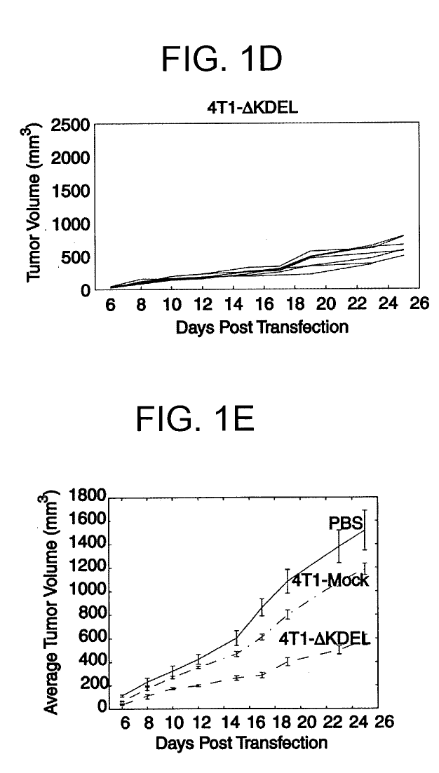

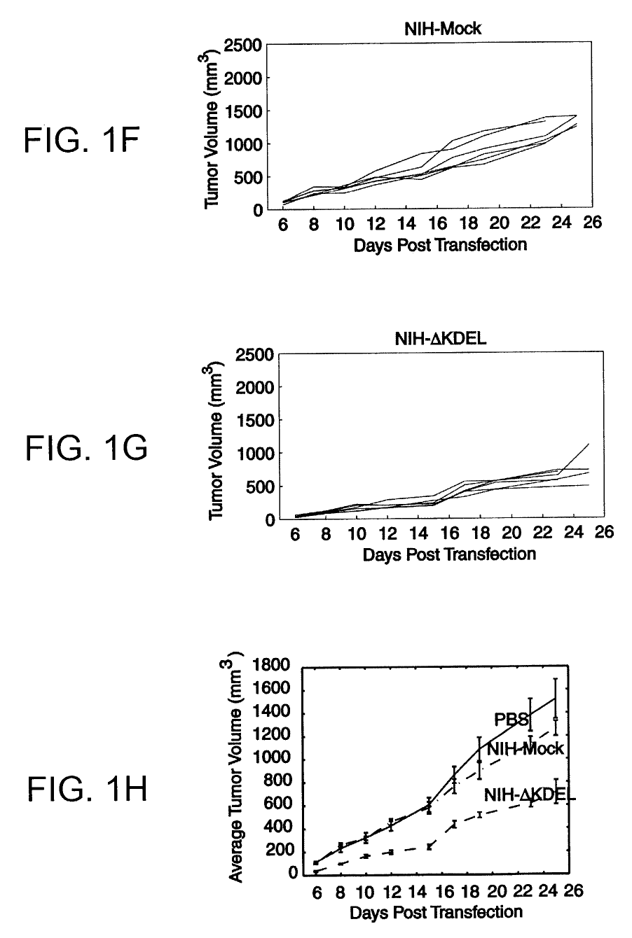

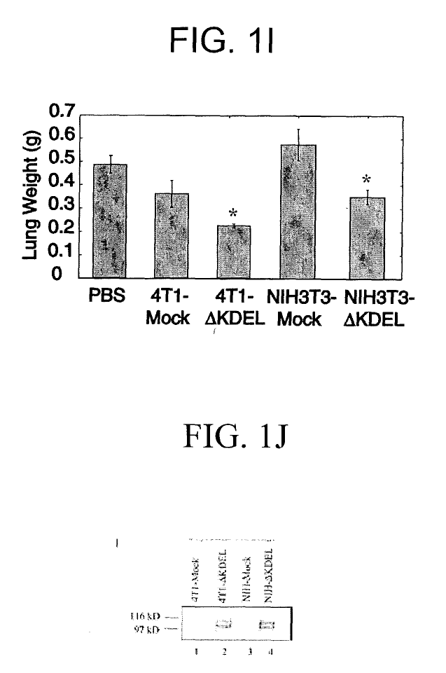

FIG. 1J shows a comparison of the relative levels of GRPΔKDEL secretion by 4T1 and NIH-3T3 cells. Equal numbers (10⁶ cells) of 4T1 or NIH3T3 cells were transfected with GRPΔKDEL (ΔKDEL samples) or mock-transfected (mock samples). 24 hours after transfection, cells were metabolically labeled with [35S] Promix and GRPΔKDEL was recovered from the media by immunoprecipitation. Proteins were resolved by SDS-PAGE on 6% gels andvisualized by Phosphorlmager analysis.

FIGS. 2A-2F demonstrate that vaccination with 4T1 mammary carcinoma cells secreting GRP(1-337) leads to delayed tumor growth rates and decreased tumor metastasis.

FIG. 2A is a picture of a polyacrylamide gel of proteins immunoprecipitated with an anti-GRP94 antibody. 4T1 cells were transfected with GRP(1-337) or with GRPΔKDEL, as indicated, or were mock-transfected (Mock). At 24 hours post-transfection, cells were metabolically labeled, conditioned chase media were collected and GRP94 domains were recovered by immunoprecipitation.

FIGS. 2B-2F are graphs depicting tumor volume (mm³) and lung weight following vaccination and tumor challenge. Female BALB/c mice were vaccinated weekly for four consecutive weeks by intradermal injection of mock-transfected 4T1 cells, GRP(1-337)-transfected 4T1 cells, or PBS (negative control). On the fifth week, animals in each group were challenged with 1×10⁶ non-irradiated 4T1 cells by intradermal injection at a remote site. Following sacrifice, lungs were resected from mice in each group and weighed as a measure of tumor metastasis. Tumor growth volume and lung weight were determined as described in Example 5.

FIG. 2B is a graph depicting tumor volume (mm³) following vaccination with PBS (negative control). Tumor volume was determined at each of the days following post-transfection, as indicated. Each line represents a growth curve for an individual subject.

FIG. 2C is a graph depicting tumor volume (mm³) following vaccination with 2-4×10⁶ mock-transfected 4T1 cells. Tumor volume was determined at each of the days following post-transfection, as indicated. Each line represents a growth curve for an individual subject.

FIG. 2D is a graph depicting tumor volume (mm³) following vaccination with 2-4×10⁶ GRP(1-337)-transfected 4T1 cells. Tumor volume was determined at each of the days following post-transfection, as indicated. Each line represents a growth curve for an individual subject.

FIG. 2E is a graph depicting average tumor volume (mm³) following vaccination with PBS (PBS, solid line), mock-transfected 4T1 cells (4T1-Mock, dashed line), or GRPΔKDEL-transfected 4T1 cells (4T1-GRP(1-337), dotted line). Tumor volume was determined at each of the days following post-transfection, as indicated.

FIG. 2F is a bar graph depicting average lung weight (g) following vaccination and tumor challenge. Asterisks indicate a significantly lower average lung weight following vaccination with GRP(1-337)-transfected 4T1 cells or when compared to controls (p=0.00031 for 4T1-GRP(1-337) by Wilcoxon rank sum test).

FIGS. 3A-3C demonstrate that GRP94ΔKDEL and GRP(1-337) elicit dendritic cell maturation following secretion from NIH3T3 fibroblast cells. Conditioned media were prepared from mock-transfected NIH3T3 cells and from NIH3T3 cells transfected with GRPΔKDEL. Conditioned media were collected for 72 hours following transfection and incubated with day 6 dendritic cells (DCs). On day 7, DCs were collected, stained with PE-conjugated anti-CD86 antibody, and analyzed by flow cytometry. Relative cell number was determined using FACSCANTM software (Becton, Dickinson & Company of Franklin Lakes, N.J., United States of America) and CELLQUESTTM software (Becton, Dickinson & Company of Franklin Lakes, N.J., United States of America) as described in Example 7.

FIG. 3A is a log plot of relative cell number of DCs incubated in media alone (dashed line) or in media plus 100 ng/ml LPS (solid line).

FIG. 3B is a log plot of relative cell number of DCs incubated in conditioned media prepared from mock-transfected NIH3T3 cells (dashed line) or in conditioned media prepared from GRPΔKDEL-transfected NIH3T3 cells (solid line).

FIG. 3C is a log plot of relative cell number of DCs incubated in conditioned media prepared from mock-transfected NIH3T3 cells (dashed line) or in conditioned media prepared from GRP(1-337)-transfected NIH3T3 cells (solid line).

FIGS. 4A-4E show that GRPΔKDEL and GRP94 NH2-terminal domain secreted by syngeneic KBALB fibroblasts yield suppression of 4T1 tumor growth and metastasis. Female BALB/c mice were immunized with PBS or with irradiated, mock-transfected, GRPΔKDEL-transfected, or GRP94 NTD-transfected KBALB fibroblasts as indicated. Animals were then challenged with unirradiated 4T1 cells as described in the Examples, and tumor volumes were followed over time. Tumor growth curves for individual mice in each group are shown in FIGS. 4A-4D and average tumor volumes with standard error are shown in FIG. 4E.

FIG. 4F shows that GRPΔKDEL or GRP94 NH2-terminal domain secretion from K-BALB fibroblasts yields decreased tumor metastasis. After animals were killed, lungs were resected from mice as shown in FIGS. 4A-4E and weighed. Average weights with standard error are shown, with groups differing significantly from PBS control denoted by an asterisk (P≦0.0003 for KBALBΔKDEL and P≦0.0002 for KBALBNTD).

FIG. 4G shows a comparison of GRPΔKDEL and GRP94 NTD secretion by 4T1 and KBALB cells. Equal numbers (10⁶ cells) of 4T1 KBALB cells were transfected with GRPΔKDEL (ΔKDEL samples), GRP94 NH2-terminal domain (NTD samples) or mock-transfected (mock samples). 24 hours after transfection, cells were metabolically labeled with [35S] Promix and GRP94 species were recovered from the media by immunoprecipitation. Proteins were resolved by SDS-PAGE on 12.5% gels and visualized by Phosphorlmager analysis.

### BRIEF DESCRIPTION OF SEQUENCES IN THE SEQUENCE LISTING

Odd-numbered SEQ ID NOs:1-21 are nucleotide sequences described in Table 1.

Even-numbered SEQ ID NOs:2-22 are protein sequences encoded by the immediately preceding nucleotide sequence, e.g., SEQ ID NO:2 is the protein encoded by the nucleotide sequence of SEQ ID NO:1, SEQ ID NO:4 is the protein encoded by the nucleotide sequence of SEQ ID NO:3, etc.

SEQ ID NO:23 is a polypeptide sequence comprising an endoplasmic reticulum retention signal.

SEQ ID NOs:24-27 are PCR primers.

TABLE 1
Sequence Listing Summary

| SEQ ID NO. | description |
|---|---|
| 1-2 | canine GRP94 N-terminal region |
| 3-4 | human HSP90 N-terminal region |
| 5-6 | canine GRP94 |
| 7-8 | human HSP90 |
| 9-10 | human HSP70 |
| 11-12 | human HSP60 |
| 13-14 | human calreticulin |
| 15-16 | canine GRP94 antigen-binding domain |
| 17-18 | human HSP90 antigen-binding domain |
| 19-20 | human HSP70 antigen-binding domain |
| 21-22 | secreted GRP94 |
| 23 | KDEL |
| 24 | primer 1 |
| 25 | primer 2 |
| 26 | primer 3 |
| 27 | primer 4 |

### DETAILED DESCRIPTION OF THE INVENTION

I. Stress Response Polypeptides

The present invention provides a recombinant stress response polypeptide free of an antigen binding domain. Also disclosed are compositions comprising a recombinant stress response polypeptide. The disclosed polypeptides are useful for eliciting immune responses, including innate and adaptive responses, as described further herein below.

The term “recombinant” generally refers to an isolated nucleic acid that is replicable in a non-native environment. Thus, a recombinant nucleic acid can comprise a non-replicable nucleic acid in combination with additional nucleic acids, for example vector nucleic acids, that enable its replication in a host cell.

The term “recombinant” as used herein also refers to a modified stress response polypeptide, wherein the modifications eliminate one or more antigen binding domains of a stress response polypeptide and/or direct its secretion from a host cell.

The terms “stress response polypeptide,” “stress response protein,” “chaperone protein,” “chaperone polypeptide,” “heat shock protein,” and “heat shock polypeptide” are used interchangeably to refer to a polypeptide involved in directing the proper folding and trafficking of newly synthesized proteins and in conferring protection to the cell during conditions of heat shock, oxidative stress, hypoxic/anoxic conditions, nutrient deprivation, other physiological stresses, and disorders or traumas that promote such stress conditions such as, for example, stroke and myocardial infarction. See e.g., Santoro (2000) Biochem Pharmacol 59:55-63; Feder & Hofmann (1999) Annu Rev Physiol 61:243-282; Robert et al. (2001) Adv Exp Med Biol 484:237-249; and Whitley et al. (1999) J Vasc Surg 29:748-751.

A recombinant stress response polypeptide of the present invention can be prepared based on the sequence of a stress response protein of any organism, including but not limited to a GRP94 polypeptide, a Hsp 90 polypeptide, a Hsp70 polypeptide, a Hsp60 polypeptide. A recombinant stress response polypeptide of the invention can also be derived from a calreticulin polypeptide. In a preferred embodiment of the invention, the recombinant stress response polypeptide comprises a recombinant GRP94 polypeptide.

The term “Hsp90 protein” refers to any of the Hsp90 class of molecular chaperones and to polypeptides substantially identical to a Hsp90 polypeptide, as defined herein below. The term “Hsp90” also encompasses any of the Grp94 class of molecular chaperones found in endoplasmic reticulum and to polypeptides substantially identical to a Grp94 polypeptide, as defined herein below.

The term “HSP90 protein” refers to an individual member of the Hsp90 class, exemplified by human HSP90, which is set forth as SEQ ID NO:8 and is encoded by a nucleic acid of SEQ ID NO:7.

The term “GRP94 protein” refers to an individual member of the Grp94 class, exemplified by canine GRP94, which is set forth as SEQ ID NO:6 and is encoded by a nucleic acid of SEQ ID NO:5.

The term “Hsp70 protein” is meant to refer to any of the Hsp70 class of molecular chaperones and to polypeptides substantially identical to a Hsp70 polypeptide, as defined herein below. A representative Hsp70 polypeptide is set forth as SEQ ID NO:10, which is encoded by a nucleic acid of SEQ ID NO:9.

The term “Hsp60 protein” is meant to refer to any of the Hsp60 class of molecular chaperones and to polypeptides substantially identical to a Hsp60 polypeptide, as defined herein below. A representative Hsp60 polypeptide is set for as SEQ ID NO:12, which is encoded by a nucleic acid of SEQ ID NO:11.

The term “calreticulin” refers to any of the class of endoplasmic reticulum proteins that comprise a calreticulin polypeptide or a polypeptide substantially identical to a calreticulin polypeptide, as defined herein below. A representative calreticulin polypeptide is set for as SEQ ID NO: 14.

I.A. Antigen Binding Domain

The present invention is markedly distinguished from current perception in the art as to the mechanism for therapy mediated by administration of a stress response polypeptide. In current views, the therapeutic activity of stress response proteins is thought to rely on the antigen binding role of the stress response protein. See e.g., Basu & Srivastava (2000) Cell Stress Chaperones 5:443-451. Recent studies have also uncovered stress response protein functions that do not require antigen binding and that appear to facilitate the antigen-specific, immunostimulatory functions of HSP-antigen complexes. However, these studies do not show or suggest a therapeutic benefit of a stress response polypeptide lacking an antigen binding domain.

Thus, the present invention provides a novel composition comprising a stress response polypeptide free of an antigen binding domain. Unexpectedly, compositions of the present invention can elicit innate and immune responses as well as other responses that reduce tumor growth and metastatic progression. While inventors do not intend to be limited to any particular theory of operation, such other responses can include an adaptive immune response.

The term “antigen” refers to a substance that activates lymphocytes (positively or negatively) by interacting with T cell or B cell receptors. Positive activation leads to immune responsiveness, and negative activation leads to immune tolerance. An antigen can comprise a protein, a carbohydrate, a lipid, a nucleic acid, or combinations thereof. An antigen can comprise a heterologous or autologous antigen (self antigen).

The term “heterologous antigen” refers to an antigen that is typically not found in a host subject. For example, an antigen derived from a pathogen is heterologous to a healthy human subject.

The term “self antigen” or “autoantigen” are used interchangeably herein and each refer to an autologous substance that behaves as an antigen. For example, necrotic cells can comprise an autologous antigen.

Heterologous and autologous antigens can further comprise an immune complex, for example a peptide that endogenously associates with a stress response protein in vivo (e.g., in infected cells or pre-cancerous or cancerous tissue). The term “antigen” can also comprise an exogenous antigen/immunogen (i.e., not complexed with GRP94 or HSP90 in vivo).

The tem “antigenic binding domain” refers to a portion of a stress response polypeptide that specifically binds an antigenic molecule. Methods for determining antigen binding activity of a stress response polypeptide are known in the art.

To assay antigen binding activity, stress response proteins can be purified from a biological sample by standard methods. See e.g., Whitley et al. (1999) J Vasc Surg 29:748-751; Walter & Blobel (1983) Methods Enzymol 96:84-93. Alternatively, stress response proteins can be recombinantly produced by heterologous expression of a nucleic acid encoding a stress response protein in a host cell.

The peptide binding activity of isolated stress response proteins can be determined by detection of bound antigens using any suitable method. For example, peptide antigens bound to purified stress response proteins can be eluted by acid extraction (Li & Srivastava, 1993), and eluted peptides can be detected by mass spectrometry. See Chapman (2000) Mass Spectrometry of Protein and Peptides. Humana Press, Totowa, N.J., United States of America. Antigens used in binding assays can also be labeled to facilitate detection of antigens bound to a stress response protein. Representative methods are described by Wearsch & Nicchitta (1997) J Biol Chem 272:5152-5156 and Suto & Srivastava (1995) Science 269:1585-1588.

An antigen binding domain of a stress response polypeptide can be mapped by analysis of recombinant stress response polypeptide variants using the peptide-binding assays summarized above. For example, stress response polypeptide fragments can be generated by expression of nucleic acids encoding a stress response polypeptide. Such modifications can include but are not limited to truncation, deletion, and mutagenesis. Standard recombinant DNA and molecular cloning techniques used to prepare nucleic acids encoding polypeptide variants are known in the art. Exemplary, non-limiting methods are described by Sambrook et al. (eds.) (1989) Molecular Cloning: A Laboratory Manual. Cold Spring Harbor Laboratory Press, Cold Spring Harbor; Silhavy et al. (1984) Experiments with Gene Fusions. Cold Spring Harbor Laboratory, Cold Spring Harbor, N.Y.; Glover & Hames (1995) DNA Cloning: A Practical Approach, 2nd ed. IRL Press at Oxford University Press, Oxford/N.Y.; Ausubel (ed.) (1995) Short Protocols in Molecular Biology, 3rd ed. Wiley, New York.

An antigen binding domain of a stress response protein can also be mapped by constructing a model based on crystallographic data of a stress response protein bound to an antigen. Programs such as RASMOL (Biomolecular Structures Group, Glaxo Wellcome Research & Development Stevenage, Hertfordshire, United Kingdom Version 2.6, August 1995, Version 2.6.4, December 1998, Copyright© Roger Sayle 1992-1999) can be used with the atomic structural coordinates from crystals generated by practicing the invention or used to practice the invention by generating three-dimensional models and/or determining the structures involved in antigen binding.

Using the methods described herein above, the antigen binding domains of several stress response proteins has been determined. For example, the peptide binding domain of GRP94 was mapped to a region near the carboxyl end of the protein (SEQ ID NO:16) (Linderoth et al., 2000). A highly conserved region was also identified in Hsp90 stress response proteins (e.g., SEQ ID NO:18).

The antigen binding domain of Hsp70 proteins and bacterial DnaK similarly maps to the carboxyl terminal half of the protein (Chappell et al., 1987; Wang et al., 1993; Gragerov et al., 1994; Zhu et al., 1996). A representative Hsp70 antigen binding domain is set forth as SEQ ID NO:20.

Based on the highly conserved nature of stress response proteins, an antigen binding domain can also be defined by determining a polypeptide domain that is substantially identical to a known antigen binding domain. Thus, a recombinant stress response polypeptide of the present invention specifically lacks an antigen binding domain, wherein the antigen binding domain binds an antigen and further comprises: (a) a polypeptide comprising an amino acid sequence of any one of even-numbered SEQ ID NOs:16-22; (b) a polypeptide substantially identical to any one of even-numbered SEQ ID NOs:16-22; (c) a polypeptide encoded by a nucleic acid of any one of odd-numbered SEQ ID NOs:15-21; or (d) a polypeptide peptide encoded by a nucleic acid substantially identical to any one of odd-numbered SEQ ID NOs:15-21. The term “substantially identical,” as used herein to describe nucleic acids and polypeptides is defined herein below.

Similarly, stress response polypeptide of the present invention can also comprise a polypeptide free of an antigen binding domain, wherein the antigen binding domain binds an antigen and further comprises a polypeptide comprising: (a) an isolated nucleic acid molecule that hybridizes to a nucleic acid comprising a nucleic acid of any one of odd-numbered SEQ ID NOs:15-21 under wash stringency conditions represented by a wash solution having less than about 200 mM salt concentration and a wash temperature of greater than about 45° C., and that encodes a GRP94 polypeptide free of an antigen binding domain; and (b) an isolated nucleic acid differing by at least one functionally equivalent codon from the isolated nucleic acid molecule of (a) above in nucleic acid sequence due to the degeneracy of the genetic code, and that encodes an antigen binding domain encoded by the isolated nucleic acid of (a) above.

I.B. Extracellular Transport

Stress response proteins can perform an immunostimulatory response when present in the extracellular milieu or expressed on the cell surface. For example, immunization of tumor-derived HSP-peptide complexes have been shown to elicit potent CTL (CD8+) and T-helper (CD4+) cell-mediated responses that result in the reduction of tumor burden (Tamura et al., 1997). In addition, treatment of antigen-presenting cells with HSP70, HSP90, or GRP94 was shown to induce potent cytokine production in macrophages (Chen et al., 1999; Kol et al., 1999; Asea et al., 2000a). Further, exogenous stress response protein is also correlated with an increased sensitivity to NK cell-mediated killing (Botzler et al., 1996a; Botzler et al., 1996b; Multhoff et al., 1997).

In a heretofore unrecognized approach, the present invention provides a recombinant stress response polypeptide that is transported extracellularly when expressed in a host cell. The host cell can comprise a cell in vivo, for example a cell in need of treatment or a cell that can assist in treatment of cells in need thereof. The host cell can also comprise a cell of a heterologous expression system, for example a cell maintained in vitro for the production of a stress response polypeptide that can be isolated and thereafter administered to a subject in need of treatment. Methods for expression of a stress response polypeptide are described further herein below.

The term “extracellular transport” refers to localization of a recombinant stress polypeptide at the cell exterior. Thus, the term “extracellular transport” encompasses insertion in a cell membrane, tethering to a cell membrane via a membranous anchor, any other association with the cell membrane, and/or secretion from a host cell.

The term “heterologous expression system” refers to a host cell comprising a heterologous nucleic acid and the polypeptide encoded by the heterologous nucleic acid. For example, a heterologous expression system can comprise a host cell transfected with a construct comprising a recombinant nucleic acid, or a cell line produced by introduction of heterologous nucleic acids into a host cell genome.

Recombinant expression of a heterologous stress response polypeptide can be variably accomplished by employing any suitable construct design, representative approaches being described herein below.

The term “recombinant” generally refers to an isolated nucleic acid that is replicable in a non-native environment. Thus, a recombinant nucleic acid can comprise a non-replicable nucleic acid in combination with additional nucleic acids, for example vector nucleic acids, that enable its replication in a host cell.

The term “vector” is used herein to refer to a nucleic acid molecule having nucleotide sequences that enable its replication in a host cell. A vector can also include nucleotide sequences to permit ligation of nucleotide sequences within the vector, wherein such nucleotide sequences are also replicated in a host cell. Representative vectors include plasmids, cosmids, and viral vectors. A vector can also mediate recombinant production of a stress response polypeptide, as described further herein below.

The term “construct”, as used herein to describe an expression construct, refers to a vector further comprising a nucleotide sequence operatively inserted with the vector, such that the nucleotide sequence is expressed. To enable expression, the nucleotide sequence to be expressed is operatively linked to a promoter region.

The term “operatively linked”, as used herein, refers to a functional combination between a promoter region and a nucleotide sequence such that the transcription of the nucleotide sequence is controlled and regulated by the promoter region. Techniques for operatively linking a promoter region to a nucleotide sequence are known in the art.

A stress response polypeptide can be expressed under the direction of any suitable promoter, including both constitutive promoters, inducible promoters, and tissue-specific promoters. Representative inducible promoters include chemically regulated promoters (e.g., the tetracycline-inducible expression system, (Gossen & Bujard, 1992; Gossen & Bujard, 1993; Gossen et al., 1995), a radiosensitive promoter (e.g., the egr-1 promoter, (Weichselbaum et al., 1994; Joki et al., 1995)), and heat-responsive promoters (Csermely et al., 1998; Easton et al., 2000; Ohtsuka & Hata, 2000). For expression of a stress response polypeptide in host cells in vivo, a tissue-specific promoter can also be used, for example the CEA promoter, which is selectively expressed in cancer cells (Hauck & Stanners, 1995; Richards et al., 1995).

A construct for expression of a stress response polypeptide of the present invention is also designed to achieve extracellular transport of the stress response polypeptide. This can be accomplished by any suitable method known in the art. Representative approaches are described herein below.

Secretion can be facilitated by mutating or eliminating portions of the heat shock protein that serve to retain the heat shock protein in the cell. For example, a sequence for retention in the endoplasmic reticulum, such as KDEL (SEQ ID NO:23) or a functionally similar sequence recognized by the erd-2 receptor, can be deleted as described in Example 1. Alternatively, retention of a stress response polypeptide in the endoplasmic reticulum can be blocked by provision of an agent that interferes with binding of the stress response polypeptide to erd-2) or by masking the retention signal sequence. See e.g., Munro & Pelham (1987) Cell 48:899-907.

A stress response polypeptide can also be targeted for extracellular transport by fusion of the encoded polypeptide to a signal peptide domain (von Heijne, 1990; Martoglio & Dobberstein, 1998; von Heijne, 1998). For example, fusion of a stress response polypeptide to an immunoglobulin Fc region can direct secretion of the polypeptide. See e.g., Yamazaki et al. (1999) J Immunol 163:5178-5182. Alternatively, a signal peptide can further comprise a transmembrane domain to direct insertion of the polypeptide in the cellular membrane. See e.g., Simonova et al. (1999) Biochem Biophys Res Commun 262:638-642 and Zheng et al. (2001) J Immunol 167:6731-6735.

Membrane localization can also be mediated by design of a stress response polypeptide comprising a domain that binds to lipid ligands embedded in the cell membrane, for example a pleckstrin homology domain, a protein kinase C homology-1 or -2 domain, and a FYVE domain. See Lemmon & Ferguson (2000) Biochem J 350 Pt 1:1-18; Johnson et al. (2000) Biochemistry 39:11360-11369; and Hurley & Misra (2000) Annu Rev Biophys Biomol Struct 29:49-79.

I.C. Polypeptides

In one embodiment, the present invention provides a construct encoding a stress response polypeptide free of an antigen binding domain. The present invention also provides a recombinantly expressed and isolated stress response polypeptide free of an antigen binding domain. Representative stress response polypeptides free of an antigen binding domain are set forth as SEQ ID NOs:2 and 4.

The term “substantially identical”, as used herein to describe a level of similarity between a stress response polypeptide and a protein substantially identical to a stress response polypeptide, refers to a sequence that is at least 35% identical to any one of even-numbered SEQ ID NOs:16-22 and that lacks an antigen binding domain. Preferably, a protein substantially identical to a stress response polypeptide comprises an amino acid sequence that is at lease about 35% to about 45% identical to any one of even-numbered SEQ ID NOs:16-22, more preferably at least about 45% to about 55% identical to any one of even-numbered SEQ ID NOs:16-22, and even more preferably at least about 55% to about 65% identical to any one of even-numbered SEQ ID NOs:16-22, wherein the polypeptide is free of an antigen binding domain. Methods for determining percent identity are defined herein below under the heading “Nucleotide and Amino Acid Sequence Comparisons.”

Substantially identical polypeptides also encompass two or more polypeptides sharing a conserved three-dimensional structure. Computational methods can be used to compare structural representations, and structural models can be generated and easily tuned to identify similarities around important active sites or ligand binding sites. See Saqi et al. (1999) Bioinformatics 15:521-522; Barton (1998) Acta Crystallogr D Biol Crystallogr 54:1139-1146; Henikoff et al. (2000) Electrophoresis 21:1700-1706; and Huang et al. (2000) Pac Symp Biocomput:230-241.

Substantially identical proteins also include proteins comprising amino acids that are functionally equivalent to amino acids of any one of even-numbered SEQ ID NOs:16-22. The term “functionally equivalent” in the context of amino acid sequences is known in the art and is based on the relative similarity of the amino acid side-chain substituents. See Henikoff & Henikoff (2000) Adv Protein Chem 54:73-97. Relevant factors for consideration include side-chain hydrophobicity, hydrophilicity, charge, and size. For example, arginine, lysine, and histidine are all positively charged residues; alanine, glycine, and serine are all of similar size; and phenylalanine, tryptophan, and tyrosine all have a generally similar shape. By this analysis, described further herein below, arginine, lysine, and histidine; alanine, glycine, and serine; and phenylalanine, tryptophan, and tyrosine; are defined herein as biologically functional equivalents.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, 1982). It is known that certain amino acids can be substituted for other amino acids having a similar hydropathic index or score and still retain a similar biological activity. In making changes based upon the hydropathic index, the substitution of amino acids whose hydropathic indices are within ±2 of the original value is preferred, those which are within ±1 of the original value are particularly preferred, and those within ±0.5 of the original value are even more particularly preferred.

It is also understood in the art that the substitution of like amino acids can be made effectively on the basis of hydrophilicity. U.S. Pat. No. 4,554,101 describes that the greatest local average hydrophilicity of a protein, as governed by the hydrophilicity of its adjacent amino acids, correlates with its immunogenicity and antigenicity, e.g., with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 of the original value is preferred, those which are within ±1 of the original value are particularly preferred, and those within ±0.5 of the original value are even more particularly preferred.

The term “substantially identical” also encompasses polypeptides that are biologically functional equivalents. The term “functional” includes activity of a stress response polypeptide free of an antigen binding domain in eliciting an immune response or an anti-cancer response, as described herein. Methods for assessing an immune response or an anti-cancer response are described in the Examples.

The present invention also provides functional fragments of a stress response polypeptide free of an antigen binding domain. For example, a functional portion need not comprise all or substantially all of an amino acid sequence of any one of even-numbered SEQ ID NOs:16-22.

The present invention also includes functional polypeptide sequences that are longer sequences than that of a stress response polypeptide free of an antigen binding domain. For example, one or more amino acids can be added to the N-terminus or C-terminus of a stress response polypeptide. Methods of preparing such proteins are known in the art.

I.D. Nucleic Acids

The terms “nucleic acid molecule” and “nucleic acid” each refer to deoxyribonucleotides or ribonucleotides and polymers thereof in single-stranded, double-stranded, or triplexed form. Unless specifically limited, the term encompasses nucleic acids containing known analogues of natural nucleotides that have similar properties as the reference natural nucleic acid. The terms “nucleic acid molecule” and “nucleic acid” can also be used in place of “gene”, “cDNA”, or “mRNA”. Nucleic acids can be synthesized, or can be derived from any biological source, including any organism.

The term “substantially identical”, as used herein to describe a degree of similarity between nucleotide sequences, refers to two or more sequences that have at least about least 60%, preferably at least about 70%, more preferably at least about 80%, more preferably about 90% to about 99%, still more preferably about 95% to about 99%, and most preferably about 99% nucleotide identity, when compared and aligned for maximum correspondence, as measured using a sequence comparison algorithm (described herein below under the heading “Nucleotide and Amino Acid Sequence Comparisons”) or by visual inspection. Preferably, the substantial identity exists in nucleotide sequences of at least about 100 residues, more preferably in nucleotide sequences of at least about 150 residues, and most preferably in nucleotide sequences comprising a full length coding sequence. The term “full length”, as used herein refers to a complete open reading frame encoding a functional stress response polypeptide free of an antigen binding domain (representative embodiments set forth as SEQ ID NOs:2 and 4. Preferred full-length nucleic acids encoding a stress response polypeptide free of an antigen binding site are set forth as SEQ ID NOs:1 and 3.

In one aspect, substantially identical sequences can comprise polymorphic sequences. The term “polymorphic” refers to the occurrence of two or more genetically determined alternative sequences or alleles in a population. An allelic difference can be as small as one base pair.

In another aspect, substantially identical sequences can comprise mutagenized sequences, including sequences comprising silent mutations. A mutation can comprise a single base change.

Another indication that two nucleotide sequences are substantially identical is that the two molecules specifically or substantially hybridize to each other under stringent conditions. In the context of nucleic acid hybridization, two nucleic acid sequences being compared can be designated a “probe” and a “target”. A “probe” is a reference nucleic acid molecule, and a “target” is a test nucleic acid molecule, often found within a heterogeneous population of nucleic acid molecules. A “target sequence” is synonymous with a “test sequence”.

A preferred nucleotide sequence employed for hybridization studies or assays includes probe sequences that are complementary to or mimic at least an about 14 to 40 nucleotide sequence of a nucleic acid molecule of the present invention. Preferably, probes comprise 14 to 20 nucleotides, or even longer where desired, such as 30, 40, 50, 60, 100, 200, 300, or 500 nucleotides or up to the full length of any one of odd-numbered SEQ ID NOs:1-21. Such probes can be readily prepared by, for example, chemical synthesis of the fragment, by application of nucleic acid amplification technology, or by introducing selected sequences into recombinant vectors for recombinant production.

The phrase “hybridizing specifically to” refers to the binding, duplexing, or hybridizing of a molecule only to a particular nucleotide sequence under stringent hybridization and wash conditions when that sequence is present in a complex nucleic acid mixture (e.g., total cellular DNA or RNA).

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

“Stringent hybridization conditions” and “stringent hybridization wash conditions” in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes, part I chapter 2, Elsevier, New York, N.Y. Generally, highly stringent hybridization and wash conditions are selected to be about 5° C. lower than the thermal melting point (Tₘ) for the specific sequence at a defined ionic strength and pH. Typically, under “stringent conditions” a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC at 65° C. An example of stringent wash conditions is 15 minutes in 0.2×SSC buffer at 65° C. See Sambrook et al., eds (1989) Molecular Cloning: A Laboratory Manual, Cold Spring Harbor Laboratory Press, Cold Spring Harbor, N.Y. for a description of SSC buffer. Often, a high stringency wash is preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4× to 6×SSC at 40° C. For short probes (e.g., about 10 to 50 nucleotides), stringent conditions typically involve salt concentrations of less than about 1 M Na⁺ ion, typically about 0.01 to 1 M Na⁺ ion concentration (or other salts) at pH 7.0-8.3, and the temperature is typically at least about 30° C. Stringent conditions can also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that can be used to identify nucleotide sequences that are substantially identical to reference nucleotide sequences of the present invention: a probe nucleotide sequence preferably hybridizes to a target nucleotide sequence in 7% sodium dodecyl sulfate (SDS), 0.5M NaPO₄, 1 mM EDTA at 50° C. followed by washing in 2×SSC, 0.1% SDS at 50° C.; more preferably, a probe and target sequence hybridize in 7% sodium dodecyl sulfate (SDS), 0.5M NaPO₄, 1 mM EDTA at 50° C. followed by washing in 1×SSC, 0.1% SDS at 50° C.; more preferably, a probe and target sequence hybridize in 7% sodium dodecyl sulfate (SDS), 0.5M NaPO₄, 1 mM EDTA at 50° C. followed by washing in 0.5×SSC, 0.1% SDS at 50° C.; more preferably, a probe and target sequence hybridize in 7% sodium dodecyl sulfate (SDS), 0.5M NaPO₄, 1 mM EDTA at 50° C. followed by washing in 0.1×SSC, 0.1% SDS at 50° C.; more preferably, a probe and target sequence hybridize in 7% sodium dodecyl sulfate (SDS), 0.5M NaPO₄, 1 mM EDTA at 50° C. followed by washing in 0.1×SSC, 0.1% SDS at 65°C.

A further indication that two nucleic acid sequences are substantially identical is that the proteins encoded by the nucleic acids are substantially identical, share an overall three-dimensional structure, or are biologically functional equivalents. These terms are defined further under the heading “Polypeptides” herein above. Nucleic acid molecules that do not hybridize to each other under stringent conditions are still substantially identical if the corresponding proteins are substantially identical. This can occur, for example, when two nucleotide sequences are significantly degenerate as permitted by the genetic code.

The term “conservatively substituted variants” refers to nucleic acid sequences having degenerate codon substitutions wherein the third position of one or more selected (or all) codons is substituted with mixed-base and/or deoxyinosine residues. See Batzer et al. (1991) Nucleic Acids Res 19:5081; Ohtsuka et al. (1985) J Biol Chem 260:2605-2608; and Rossolini et al. (1994) Mol Cell Probes 8:91-98

The term “subsequence” refers to a sequence of nucleic acids that comprises a part of a longer nucleic acid sequence. An exemplary subsequence is a probe, described herein above, or a primer. The term “primer” as used herein refers to a contiguous sequence comprising about 8 or more deoxyribonucleotides or ribonucleotides, preferably 10-20 nucleotides, and more preferably 20-30 nucleotides of a selected nucleic acid molecule. The primers of the invention encompass oligonucleotides of sufficient length and appropriate sequence so as to provide initiation of polymerization on a nucleic acid molecule of the present invention.

The term “elongated sequence” refers to a sequence comprising additional nucleotides (or other analogous molecules) incorporated into and/or at either end of a nucleic acid. For example, a polymerase (e.g., a DNA polymerase) can add sequences at the 3′ terminus of a nucleic acid molecule. In addition, a nucleotide sequence can be combined with other DNA sequences, such as promoters, promoter regions, enhancers, polyadenylation signals, intronic sequences, additional restriction enzyme sites, multiple cloning sites, and other coding segments.

The term “complementary sequences”, as used herein, indicates two nucleotide sequences that comprise antiparallel nucleotide sequences capable of pairing with one another upon formation of hydrogen bonds between base pairs. As used herein, the term “complementary sequences” means nucleotide sequences which are substantially complementary, as can be assessed by the same nucleotide comparison set forth above, or is defined as being capable of hybridizing to the nucleic acid segment in question under relatively stringent conditions such as those described herein. An example of a complementary nucleic acid segment is an antisense oligonucleotide.

The term “gene” refers broadly to any segment of DNA associated with a biological function. A gene encompasses sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

Nucleic acids of the present invention can be cloned, synthesized, recombinantly altered, mutagenized, or combinations thereof. Standard recombinant DNA and molecular cloning techniques used to isolate nucleic acids are known in the art. Site-specific mutagenesis to create base pair changes, deletions, or small insertions are also known in the art as exemplified by publications. See e.g., Sambrook et al. (eds.) (1989) Molecular Cloning: A Laboratory Manual. Cold Spring Harbor Laboratory Press, Cold Spring Harbor; Silhavy et al. (1984) Experiments with Gene Fusions. Cold Spring Harbor Laboratory, Cold Spring Harbor, N.Y.; Glover & Hames (1995) DNA Cloning: A Practical Approach, 2nd ed. IRL Press at Oxford University Press, Oxford/N.Y.; and Ausubel (ed.) (1995) Short Protocols in Molecular Biology, 3rd ed. Wiley, New York.

I.E. Nucleotide and Amino Acid Sequence Comparisons

The terms “identical” or percent “identity” in the context of two or more nucleotide or polypeptide sequences, refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term “substantially identical” in regards to a nucleotide or polypeptide sequence means that a particular sequence varies from the sequence of a naturally occurring sequence by one or more deletions, substitutions, or additions, the net effect of which is to retain biological activity of a gene, gene product, or sequence of interest.

For sequence comparison, typically one sequence acts as a reference sequence to which test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison can be conducted, e.g., by the local homology algorithm of Smith & Waterman (1981) Adv Appl Math 2:482-489, by the homology alignment algorithm of Needleman & Wunsch (1970) J Mol Biol 48:443-453, by the search for similarity method of Pearson & Lipman (1988) Proc Natl Acad Sci USA 85:2444-2448, by computerized implementations of these algorithms (GAP, BESTFIT, FASTA, and TFASTA in the Wisconsin Genetics Software Package, Genetics Computer Group, Madison, Wis.), or by visual inspection. See generally, Ausubel (ed.) (1995) Short Protocols in Molecular Biology, 3rd ed. Wiley, New York.

A preferred algorithm for determining percent sequence identity and sequence similarity is the BLAST algorithm, which is described by Altschul et al. (1990) J Mol Biol 215:403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=4, and a comparison of both strands. For amino acid sequences, the BLASTP program uses as defaults a wordlength (W) of 3, an expectation (E) of 10, and the BLOSUM62 scoring matrix. See Henikoff & Henikoff (1992) Proc Natl Acad Sci USA 89:10915-10919.

In addition to calculating percent sequence identity, the BLAST algorithm also performs a statistical analysis of the similarity between two sequences. See e.g., Karlin & Altschul (1993) Proc Natl Acad Sci USA 90:5873-5877. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

II. Therapeutic Applications

The present invention provides therapeutic compositions comprising a recombinant stress response polypeptide free of an antigen binding domain. Provision of a recombinant stress response polypeptide lacking an antigen binding domain can elicit an innate immune response, as described in Example 7. Administration to a subject of a recombinant stress response polypeptide can also elicit and adaptive immune response in the subject, the specificity of the response directed to antigens present in the subject or to exogenously provided antigens (Example 6).

The compositions of the present invention can also be used to elicit an anti-cancer response in a subject via administration of the stress response polypeptide to the subject. While applicants do not intend to be bound to any particular theory of operation, an “anti-cancer response” can comprise an immune response, an anti-angiogenic response, or a combination thereof. See Example 6.

The methods of the present invention involve administering a stress response polypeptide extracellularly. In one embodiment of the invention, the administering comprises administering a gene therapy construct encoding a stress response polypeptide, wherein the stress response polypeptide is designed for extracellular transport, as described herein above. In another embodiment of the invention, a stress response polypeptide is produced in a heterologous expression system, purified from the expression system, and formulated for administration. Representative methods for heterologous expression and formulation are also described herein above.

The term “immune system” includes all the cells, tissues, systems, structures and processes, including non-specific and specific categories, that provide a defense against cells comprising antigenic molecules, including but not limited to tumors, pathogens, and self-reactive cells. Thus, an immune response can comprise an innate immune response, an adaptive immune response, or a combination thereof.

The term “innate immune system” includes phagocytic cells such as neutrophils, monocytes, tissue macrophages, Kupffer cells, alveolar macrophages, dendritic cells, and microglia. The innate immune system mediates non-specific immune responses. The innate immune system plays an important role in initiating and guiding responses of the adaptive immune system. See e.g., Janeway (1989) Cold Spring Harb Symp Quant Biol 54:1-13; Romagnani (1992) Immunol Today 13:379-381; Fearon & Locksley (1996) Science 272:50-53; and Fearon (1997) Nature 388:323-324. An innate response can comprise, for example, dendritic cell maturation, macrophage activation, cytokine or chemokine secretion, and/or activation of NFκB signaling.

The term “adaptive immune system” refers to the cells and tissues that impart specific immunity within a host. Included among these cells are natural killer (NK) cells and lymphocytes (e.g., B cell lymphocytes and T cell lymphocytes). The term “adaptive immune system” also includes antibody-producing cells and the antibodies produced by the antibody-producing cells.

The term “adaptive immune response” refers to a specific response to an antigen include humoral immune responses (e.g., production of antigen-specific antibodies) and cell-mediated immune responses (.e.g., lymphocyte proliferation), as defined herein below. An adaptive immune response can further comprise systemic immunity and humoral immunity.

The terms “cell-mediated immunity” and “cell-mediated immune response” are meant to refer to the immunological defense provided by lymphocytes, such as that defense provided by T cell lymphocytes when they come into close proximity to their victim cells. A cell-mediated immune response also comprises lymphocyte proliferation. When “lymphocyte proliferation” is measured, the ability of lymphocytes to proliferate in response to specific antigen is measured. Lymphocyte proliferation is meant to refer to B cell, T-helper cell or CTL cell proliferation.

The term “CTL response” is meant to refer to the ability of an antigen-specific cell to lyse and kill a cell expressing the specific antigen. As described herein below, standard, art-recognized CTL assays are performed to measure CTL activity.

The term “systemic immune response” is meant to refer to an immune response in the lymph node-, spleen-, or gut-associated lymphoid tissues wherein cells, such as B lymphocytes, of the immune system are developed. For example, a systemic immune response can comprise the production of serum IgG's. Further, systemic immune response refers to antigen-specific antibodies circulating in the blood stream and antigen-specific cells in lymphoid tissue in systemic compartments such as the spleen and lymph nodes.

The terms “humoral immunity” or “humoral immune response” are meant to refer to the form of acquired immunity in which antibody molecules are secreted in response to antigenic stimulation.

Thus, the compositions of the present invention can enhance the immunocompetence of a subject and elicit specific immunity against antigens associated with diseases and disorders including but not limited to cancer, infection, angiogenic disorders, and cellular necrosis. The present invention also pertains to administration of a stress response polypeptide free of an antigen binding domain to a subject at risk of developing any of the foregoing diseases and disorders due to familial history or environmental factors.

A recombinant stress response polypeptide of the present invention is further useful for cellular immunotherapies, including any adoptive immunotherapeutic approach involving ex vivo preparation of cells of the innate immune system.

A recombinant stress response polypeptide of the present invention is further useful as an adjuvant for eliciting a specific immune response to an exogenous antigen.

II.A. Subjects

The term “subject” as used herein includes any vertebrate species, preferably warm-blooded vertebrates such as mammals and birds. More particularly, the methods of the present invention are contemplated for the treatment of tumors in mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants and livestock (such as cattle, oxen, sheep, giraffes, deer, goats, bison, and camels), and horses. Also contemplated is the treatment of birds, including those kinds of birds that are endangered or kept in zoos, as well as fowl, and more particularly domesticated fowl or poultry, such as turkeys, chickens, ducks, geese, guinea fowl, and the like, as they are also of economical importance to humans.

II.B. Monitoring Immune Response

Methods for monitoring an immune response in a subject are known to one skilled in the art. Representative methods that can be used as general indicators of an immunostimulatory response are described herein below. Additional methods suitable for assessment of particular therapies or applications can also be used.

Delayed Hypersensitivity Skin Test. Delayed hypersensitivity skin tests are of great value in the overall immunocompetence and cellular immunity to an antigen. Inability to react to a battery of common skin antigens is termed anergy (Sato et al. (1995) Clin Immunol Pathol 74:35-43). Proper technique of skin testing requires that the antigens be stored sterile at 4° C., protected from light and reconstituted shortly before use. A 25- or 27-gauge needle ensures intradermal, rather than subcutaneous, administration of antigen. Twenty-four and forty-eight hours after intradermal administration of the antigen, the largest dimensions of both erythema and induration are measured with a ruler. Hypoactivity to any given antigen or group of antigens is confirmed by testing with higher concentrations of antigen or, in ambiguous circumstances, by a repeat test with an intermediate concentration.

Activity of Cytolytic T-lymphocytes In vitro. 8×10⁶ peripheral blood derived T lymphocytes isolated by the Ficoll-Hypaque centrifugation gradient technique, are re-stimulated with 4×10⁴ mitomycin C treated tumor cells in 3 ml RPMI medium containing 10% fetal calf serum. In some experiments, 33% secondary mixed lymphocyte culture supernatant or IL-2, is included in the culture medium as a source of T cell growth factors.

To measure the primary response of cytolytic T-lymphocytes after immunization, T cells are cultured without the stimulator tumor cells. In other experiments, T cells are re-stimulated with antigenically distinct cells. After six days, the cultures are tested for cytotoxicity in a 4 hour ⁵¹Cr-release assay. The spontaneous ⁵¹Cr-release of the targets preferably reaches a level less than 20%. To determine anti-MHC class I blocking activity, a ten-fold concentrated supernatant of W6/32 hybridoma is added to the test at a final concentration of about 12.5% (Heike et al. (1994) J Immunotherapy 15:165-174).

Levels of Cell-Specific Antigens. Monitoring of disease and infection can also be accomplished using any one of a variety of biochemical techniques that assay a level of antigen whose presence is indicative of disease or infection.

For example, carcinoembryonic antigen (CEA) is a glycoprotein found on human colon cancer cells, but not on normal adult colon cells. Subjects with other tumors, such as pancreatic and breast cancer, also have elevated serum levels of CEA. Therefore, monitoring the fall and rise of CEA levels in cancer patients undergoing therapy has proven useful for predicting tumor progression and responses to treatment. Similarly, serum levels of prostate-specific antigen (PSA) are indicative of a risk for developing prostrate cancer.

Immunodiagnostic methods can be used to detect antigens present on pathogens present in infected cells. For example, a pathogen-specific antigen can comprise a polypeptide that mediates disease progression, i.e. toxic shock syndrome toxin-1 or an enterotoxin.

Gene Expression. Disease and infection can also be monitored by detection of a nucleic acid presence or amount that is characteristic to disease or infection. Formats for assaying gene expression can include but are not limited to PCR amplification of a target nucleic acid and hybridization-based methods of nucleic acid detection. These assays can detect the presence and/or level of a single target nucleic acid or multiple target nucleic acids, for example by microarray analysis.

Target-specific probes can be designed according to nucleotide sequences in public sequence repositories (e.g., Sanger Centre (ftp://ftp.sanger.ac.uk/pub/tb/sequences) and GenBank (http://ncbi.nlm.nih.gov)), including cDNAs, expressed sequence tags (ESTs), sequence tagged sites (STSs), repetitive sequences, and genomic sequences.

Representative methods for detection of nucleic acids and the selection of appropriate target genes are described in, for example, Quinn (1997) in Lee et al., eds., Nucleic Acid Amplification Technologies: Application to Disease Diagnostics, pp.49-60, Birkhäuser Boston, Cambridge, Mass., United States of America; Richardson & Warnock (1993) Fungal Infection: Diagnosis and Management, Blackwell Scientific Publications Inc., Boston, Mass., United States of America; Storch (2000) Essentials of Diagnostic Virology, Churchill Livingstone, New York, N.Y.; Fisher & Cook (1998) Fundamentals of Diagnostic Mycology, W.B. Saunders Company, Philadelphia, Pa.; White & Fenner (1994) Medical Virology, 4^{th} Edition, Academic Press, San Diego, Calif.; and Schena (2000) Microarray Biochip Technology. Eaton Publishing, Natick, Mass., United States of America.

II.C. Treatment of Cancer and Other Proliferative Disorders

The present invention provides a method for inhibiting cancer growth via administration of a stress response polypeptide free of an antigen binding domain. See Example 6.

The term “cancer” as used herein generally refers to tumors, neoplastic cells and preneoplastic cells, and other disorders of cellular proliferation.

The term “tumor” encompasses both primary and metastasized solid tumors and carcinomas of any tissue in a subject, including but not limited to breast; colon; rectum; lung; oropharynx; hypopharynx; esophagus; stomach; pancreas; liver; gallbladder; bile ducts; small intestine; urinary tract including kidney, bladder and urothelium; female genital tract including cervix, uterus, ovaries (e.g., choriocarcinoma and gestational trophoblastic disease); male genital tract including prostate, seminal vesicles, testes and germ cell tumors; endocrine glands including thyroid, adrenal, and pituitary; skin (e.g., hemangiomas and melanomas), bone or soft tissues; blood vessels (e.g., Kaposi's sarcoma); brain, nerves, eyes, and meninges (e.g., astrocytomas, gliomas, glioblastomas, retinoblastomas, neuromas, neuroblastomas, Schwannomas and meningiomas). The term “tumor” also encompasses solid tumors arising from hematopoietic malignancies such as leukemias, including chloromas, plasmacytomas, plaques and tumors of mycosis fungoides and cutaneous T-cell lymphoma/leukemia, and lymphomas including both Hodgkin's and non-Hodgkin's lymphomas.

The term “neoplastic cell” refers to new and abnormal cell. The term “neoplasm” encompasses a tumor.

The term “preneoplastic” cell refers to a cell which is in transition from a normal to a neoplastic form.

The compositions of the present invention can also be use for the treatment or prevention of non-neoplastic cell growth such as hyperplasia, metaplasia, and dysplasia. See Kumar et al. (1997) Basic Pathology, 6th ed. W.B. Saunders Co., Philadelphia, Pa., United States of America.

The term “hyperplasia” refers to an abnormal cell proliferation involving an increase in cell number in a tissue or organ, without significant alteration in structure or function. As one example, endometrial hyperplasia often precedes endometrial cancer.

The term “metaplasia” refers to abnormal cell growth in which one type of adult or fully differentiated cell substitutes for another type of adult cell. Metaplasia can occur in epithelial or connective tissue cells. Atypical metaplasia can result in a disordered metaplastic epithelium.

The term “dysplasia” refers to abnormal cell proliferation involving a loss in individual cell uniformity and in the architectural orientation of cells. Dysplastic cells often have abnormally large, deeply stained nuclei, and exhibit pleomorphism. Dysplasia is frequently a forerunner of cancer, and is found mainly in the epithelia of irritated or inflamed tissues including the cervix, respiratory passages, oral cavity, and gall bladder.

Administration of a recombinant stress response polypeptide free of an antigen binding site can be combined with conventional cancer therapies. For example, administration of composition of the present invention can be used to minimize infection and other complications resulting from immunosuppression. The therapeutic methods disclosed herein are also useful for controlling metastases, for example metastases arising from tumor cells shed into the circulation during surgical removal of a tumor.

The term “cancer growth” generally refers to any one of a number of indices that suggest change within the cancer to a more developed form. Thus, indices for measuring an inhibition of cancer growth include but are not limited to a decrease in cancer cell survival, a decrease in tumor volume or morphology (for example, as determined using computed tomographic (CT), sonography, or other imaging method), a delayed tumor growth, a destruction of tumor vasculature, improved performance in delayed hypersensitivity skin test, an increase in the activity of cytolytic T-lymphocytes, and a decrease in levels of tumor-specific antigens.

The term “delayed tumor growth” refers to a decrease in a duration of time required for a tumor to grow a specified amount. For example, treatment can delay the time required for a tumor to increase in volume 3-fold relative to an initial day of measurement (day 0) or the time required to grow to 1 cm³.

II.D. Treatment of Infection

The compositions of the present invention can also be used to enhance an immune response against cells infected with an antigen. Thus, the present invention provides a method for eliciting an immune response in a subject, wherein the immune response comprises an anti-pathogen response, via administration of a stress response polypeptide free of an antigen binding domain.

The term “pathogen” and “infectious agent” are used interchangeably herein to refer to a bacterium, a virus, a fungus, a protozoan, a parasite, other infective agent, or potentially harmful or parasitic organism. Normal microbial flora are also potential pathogens.

Representative bacterial infectious that can be treated or prevented using the methods of the present invention include but are not limited to those infections caused by species of the genera Salmonella, Shigella, Actinobacillus, Porphyromonas, Staphylococcus, Bordetella, Yersinia, Haemophilus, Streptococcus, Chlamydophila, Alliococcus, Campylobacter, Actinomyces, Neisseria, Chlamydia, Treponema, Ureaplasma, Mycoplasma, Mycobacterium, Bartonella, Legionella, Ehrlichia, Escherichia, Listeria, Vibrio, Clostridium, Tropheryma, Actinomadura, Nocardia, Streptomyces, and Spirochaeta.

Representative viral infections that can be treated or prevented by the methods of the present invention include but are not limited to those infections caused by DNA viruses, such as Poxviridae, Herpesviridae, Adenoviridae, Papoviridae, Hepadnaviridae, and Parvoviridae. RNA viruses are also envisioned to be detected in accordance with the disclosed methods, including Paramyxoviridae, Orthomyxoviridae, Coronaviridae, Arenaviridae, Retroviridae, Reoviridae, Picornaviridae, Caliciviridae, Rhabdoviridae, Togaviridae, Flaviviridae, and Bunyaviridae.

Representative viruses include but are not limited to, hepatitis viruses, flaviviruses, gastroenteritis viruses, hantavi ruses, Lassa virus, Lyssavirus, picornaviruses, polioviruses, enteroviruses, nonpolio enteroviruses, rhinoviruses, astroviruses, rubella virus, HIV-1 (human immunodeficiency virus type 1), HIV-2 (human immunodeficiency virus type 2), HTLV-1 (human T-lymphotropic virus type 1), HTLV-2 (human T-lymphotropic virus type 2), HSV-1 (herpes simplex virus type 1), HSV-2 (herpes simplex virus type 2), VZV (varicellar-zoster virus), CMV (cytomegalovirus), HHV-6 (human herpes virus type 6), HHV-7 (human herpes virus type 7), EBV (Epstein-Barr virus), influenza A and B viruses, adenoviruses, RSV (respiratory syncytial virus), PIV-1 (parainfluenza virus, types 1, 2, and 3), papillomavirus, JC virus, polyomaviruses, BK virus, filoviruses, coltiviruses, orbiviruses, orthoreoviruses, retroviruses, and spumaviruses.

Representative fungal infections that can be treated or prevented using the methods of the present invention include but are not limited to those infections caused by species of the genera Aspergillus, Trichophyton, Microsporum, Epidermaophyton, Candida, Malassezia, Pityrosporum, Trichosporon, Exophiala, Cladosporium, Hendersonula, Scytalidium, Piedraia, Scopulariopis, Acremonium, Fusarium, Curvularia, Penicillium, Absidia, Pseudallescheria, Rhizopus, Cryptococcus, MuCunninghamella, Rhizomucor, Saksenaea, Blastomyces, Coccidioides, Histoplasma, Paraoccidioides, Phialophora, Fonsecaea, Rhinocladiella, Conidiobolu, Loboa, Leptosphaeria, Madurella, Neotestudina, Pyrenochaeta, Colletotrichum, Alternaria, Bipolaris, Exserohilum, Phialophora, Xylohypha, Scedosporium, Rhinosporidium, and Sporothrix.

Protozoal infections that can be treated or prevented by the methods of the present invention include but are not limited to those infections caused by species of the genera Toxoplasma, Giardia, Cryptosporidium, Trichomonas, and Leishmania. Other infections that can be treated or prevented by the methods of the present invention include but are not limited to those infections caused by parasitic species of the genera Rickettsiae and by nematodes such as species of the genera Trichinella and Anisakis.

II.E. Treatment of Angiogenic Disorders

The present invention further provides compositions and methods useful for the treatment or prevention of angiogenic disorders. The method comprises administering to a subject an effective amount of a stress response polypeptide free of an antigen binding domain, whereby blood vessel growth is inhibited.

The term “angiogenesis” refers to the process by which new blood vessels are formed. The term “anti-angiogenic response” and “anti-angiogenic activity” as used herein, each refer to a biological process wherein the formation of new blood vessels is inhibited.

Methods for assaying a level of angiogenesis include determining vascular length and microvessel density. Representative methods are described by Hironaka et al. (2002) Clin Cancer Res 8:124-130; Starnes et al. (2000) J Thorac Cardiovasc Surg 120:902-907; and El-Assal et al. (1998) Hepatology 27:1554-1562.

Angiogenesis can also be monitored by measuring blood flow. For example, Power Doppler sonography utilizes amplitude to measure flow in microvasculature. Tissues can be imaged with a 10-5 MHz ENTOS® linear probe (Advanced Technology Laboratories, Inc. of Bothell, Wash., United States of America) attached to an HDI® 5000 diagnostic ultrasound system (Advanced Technology Laboratories, Inc. of Bothell, Wash., United States of America).

II.F. Treatment of Cellular Necrosis

Also provided is a method for treating cellular necrosis resulting from cellular injury, disease, or other conditions such as ischemia/reperfusion. The method comprises administering to a subject an effective amount of a stress response polypeptide free of an antigen binding domain, whereby cellular necrosis is abrogated.

The term “cellular necrosis” refers to cell death caused by disease, physical or chemical injury, or ischemia.

The term “ischemia” refers to a loss of blood flow to a tissue. Blood loss is characterized by deprivation of both oxygen and glucose, and leads to ischemic necrosis or infarction. Thus, the term “ischemia” refers to both conditions of oxygen deprivation and of nutrient deprivation. Loss of blood flow to a particular vascular region is described as “focal ischemia”. Loss of blood flow to an entire tissue or body is referred to as “global ischemia”.

The present invention provides therapeutic compositions and methods to ameliorate cellular damage arising from conditions of ischemia/reperfusion including but not limited to cardiac arrest, asystole and sustained ventricular arrythmias, cardiac surgery, cardiopulmonary bypass surgery, organ transplantation, spinal cord injury, head trauma, stroke, thromboembolic stroke, hemorrhagic stroke, cerebral vasospasm, hypotension, hypoglycemia, status epilepticus, an epileptic seizure, anxiety, schizophrenia, a neurodegenerative disorder, Alzheimer's disease, Huntington's disease, amyotrophic lateral sclerosis (ALS), neonatal stress, and any condition in which a neuroprotectant composition that prevents or ameliorates ischemic cerebral damage is indicated, useful, recommended, or prescribed.

II.G. Cellular Immunotherapy

The present invention further provides compositions and methods for cellular immunotherapy. The term “cellular immunotherapy” refers to preparation of cells for administration to a subject to thereby elicit an immune response, including an anti-tumor response.

In one embodiment of the invention, compositions and methods are provided for administering healthy cells expressing a soluble stress response protein to a subject. The term “healthy,” as used herein to describe a cellular carrier for immunotherapy, comprises a cell other than a cell to be treated. Representative healthy cells include but are not limited to non-cancerous cells, cells free of a pathogen, and non-necrotic cells. The cells can be autologous or heterologous (e.g., allogenic) to a subject in need of treatment.

For example, a construct encoding a secreted stress response protein can be prepared as described herein above. A representative secreted stress response polypeptide is set forth as SEQ ID NO:22. The construct is transfected into healthy cells, which are then administered to a subject to thereby treat an infection or disease. In a preferred embodiment of the invention, the treatment response comprises an anti-tumor response and/or an anti-metastatic response, as described in Example 5.

In another embodiment of the invention, compositions and methods are provided for preparing antigen presenting cells (APCs) useful for adoptive immunotherapies. The term “adoptive immunotherapy” as used herein refers to a therapeutic approach whereby antigen-presenting cells are prepared ex vivo and then administered to a subject in need of treatment. See Example 7.

Antigen-presenting cells, including but not limited to macrophages, dendritic cells and B-cells, can be obtained by production in vitro from stem and from progenitor cells found in human peripheral blood and bone marrow. See Inaba (1992) J Exp Med 176:1693-1702. Preferably, the subject into which the sensitized APCs are injected is the subject from which the APC were originally isolated (autologous embodiment).

The present invention provides a method for preparing sensitized APCs via exposing APCs to stress response polypeptide free of an antigen binding domain and a danger signal of interest. For example, sensitized DCs can be prepared by exposing immature DCs to a stress response polypeptide of the present invention and to an antigen against which a specific immune response is sought.

Sensitized APCs are re-infused into a subject systemically, preferably intravenously, by conventional clinical procedures. Subjects generally receive from about 10⁶ to about 10¹² sensitized APCs, depending on the condition of the subject and the condition to be treated. In some regimens, subjects can optionally receive in addition a suitable dosage of a biological response modifier including but not limited to the cytokines IFN-α, IFN-γ, IL-2, IL-4, IL-6, TNF or other cytokine growth factor.

II.H. Adjuvant Activity

A stress response polypeptide free of an antigen binding domain can also be used as an adjuvant to promote a specific immune response against an exogenous antigen. For example, an exogenous and a recombinant stress response polypeptide of the present invention can be co-administered to a subject, whereby the specificity of an adaptive immune response in the subject is directed to the antigen.

The term “adjuvant activity” is meant to refer to a molecule having the ability to enhance or otherwise modulate the response of a vertebrate subject's immune system to an antigen.

Adjuvants can be used to improve the activity of vaccine antigens by modulating immune responses, including (1) stimulating humoral and cell mediated immunity; (2) eliciting cytokine and chemokine production by APCs; and (3) controlling the type of acquired immune response that is induced (Yip et al., 1999). See O'Hagan et al. (2001) Biomol Eng 18:69-85.

Antigens can be selected for use from among those known in the art or determined by immunoassay to be antigenic or immunogenic. The term “antigenic” refers to a quality of binding to an antibody or to a MHC molecule. The term “immunogenic” refers to a quality of eliciting an immune response.

Antigenicity of a candidate antigen can be determined by various immunoassays known in the art, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in vivo immunoassays (using colloidal gold, enzyme or radioisotope labels, for example), western blots, immunoprecipitation reactions, agglutination assays (e.g., gel agglutination assays, hemagglutination assays), complement fixation assays, immunofluorescence assays, protein A assays, and immuno-electrophoresis assays.

Immunogenicity can be determined by, for example, detecting T cell-mediated responses. Representative methods for measuring T cell responses include in vitro cytotoxicity assays or in vivo delayed-type hypersensitivity assays, as described herein above. Immunogenicity can also be assessed by detection of antigen-specific antibodies in a subject's serum, and/or by a demonstration of protective effects of antisera or immune cells specific for the antigen.

Candidate immunogenic or antigenic peptides can be isolated from either endogenous stress response protein-antigen complexes as described or from endogenous MHC-peptide complexes for use subsequently as antigenic molecules. The isolation of potentially immunogenic peptides from MHC molecules is well known in the art. See Falk et al. (1990) Nature 348:248-251; Rotzschke et al. (1990) Nature 348:252-254; Falk et al. (1991) Nature 351:290-296; Elliott et al. (1990) Nature 348:195-197; Demotz et al. (1989) Nature 342:682-684; and Rotzschke et al. (1990) Science 249:283-287.

Potentially useful antigens can also be identified by various criteria, such as the antigen's involvement in neutralization of a pathogen's infectivity (wherein it is desired to treat or prevent infection by such a pathogen). See Norrby & Cold Spring Harbor Laboratory. (1994) Vaccines 94: Modern Approaches to New Vaccines Including Prevention of Aids. Cold Spring Harbor Laboratory Press, Plainview, N.Y.

Preferably, where it is desired to treat or prevent cancer, known tumor-specific antigens or fragments or derivatives thereof are used. For example, such tumor-specific or tumor-associated antigens include but are not limited to KS 1/4 pan-carcinoma antigen (Bumol et al., 1988; Perez & Walker, 1989); ovarian carcinoma antigen (CA125) (Yu & Lian, 1991); prostatic acid phosphate (Tailor et al., 1990); prostate specific antigen (Henttu & Vihko, 1989; Israeli et al., 1993); melanoma-associated antigen p97 (Estin et al., 1989); melanoma antigen gp75 (Vijayasaradhi et al., 1990); high molecular weight melanoma antigen (Natali et al., 1987); and prostate specific membrane antigen (Mai et al., 2000).

Preferably, where it is desired to treat or prevent viral diseases, molecules comprising epitopes of known viruses are used. For example, such antigenic epitopes can be prepared from viruses including any of the viruses noted herein above.

Preferably, where it is desired to treat or prevent bacterial infections, molecules comprising epitopes of known bacteria are used including but not limited to any of the bacteria noted herein above.

Preferably, where it is desired to treat or prevent protozoan or parasitic infectious, molecules comprising epitopes of known protozoa or parasites are used. For example, such antigenic epitopes can be prepared from any protozoa or parasite, including any of those noted herein above.

An antigen to be co-administered with a stress response polypeptide of the invention can also comprise any other antigen to which an immune response is desired. A stress response polypeptide free of an antigen binding domain can be particularly useful for eliciting immune responses to poorly immunogenic antigens.

III. Therapeutic Compositions and Methods

In accordance with the methods of the present invention, a composition that is administered to elicit an immune response in a subject comprises: (a) an immunostimulatory amount of a stress response polypeptide free of an antigen binding domain; and (b) a pharmaceutically acceptable carrier.

III.A. Carriers

Any suitable carrier that facilitates drug preparation and/or administration can be used. The carrier can be a viral vector or a non-viral vector. Suitable viral vectors include adenoviruses, adeno-associated viruses (AAVs), retroviruses, pseudotyped retroviruses, herpes viruses, vaccinia viruses, Semiliki forest virus, and baculoviruses. In a preferred embodiment of the invention, the carrier comprises an adenoviral gene therapy construct that encodes a stress response protein.

Suitable non-viral vectors that can be used to deliver a stress response protein include but are not limited to a plasmid, a nanosphere (Manome et al., 1994; Saltzman & Fung, 1997), a peptide (U.S. Pat. Nos. 6,127,339 and 5,574,172), a glycosaminoglycan (U.S. Pat. No. 6,106,866), a fatty acid (U.S. Pat. No. 5,994,392), a fatty emulsion (U.S. Pat. No. 5,651,991), a lipid or lipid derivative (U.S. Pat. No. 5,786,387), collagen (U.S. Pat. No. 5,922,356), a polysaccharide or derivative thereof (U.S. Pat. No. 5,688,931), a nanosuspension (U.S. Pat. No. 5,858,410), a polymeric micelle or conjugate (Goldman et al., 1997) and U.S. Pat. Nos. 4,551,482, 5,714,166, 5,510,103, 5,490,840, and 5,855,900), and a polysome (U.S. Pat. No. 5,922,545).

Where appropriate, two or more types of carriers can be used together. For example, a plasmid vector can be used in conjunction with liposomes. Currently, a preferred embodiment of the present invention envisions the use of an adenovirus.

A carrier can be selected to effect sustained bioavailability of a recombinant stress response polypeptide to a site in need of treatment. The term “sustained bioavailability” is used herein to refer to a bioavailability of a stress response polypeptide free of an antigen binding domains sufficient to elicit an immune response. The term “sustained bioavailability” also refers to a bioavailability of a stress response polypeptide of the present invention sufficient to inhibit blood vessel growth within a tumor. The term “sustained bioavailability” encompasses factors including but not limited to prolonged release of a stress response polypeptide from a carrier, metabolic stability of a stress response polypeptide, systemic transport of a composition comprising a stress response polypeptide, and effective dose of a stress response polypeptide.

Representative compositions for sustained bioavailability of stress response polypeptide can include but are not limited to polymer matrices, including swelling and biodegradable polymer matrices, (U.S. Pat. Nos. 6,335,035; 6,312,713; 6,296,842; 6,287,587; 6,267,981; 6,262,127; and 6,221,958), polymer-coated microparticles (U.S. Pat. Nos. 6,120,787 and 6,090,925) a polyol:oil suspension (U.S. Pat. No. 6,245,740), porous particles (U.S. Pat. No. 6,238,705), latex/wax coated granules (U.S. Pat. No. 6,238,704), chitosan microcapsules, and microsphere emulsions (U.S. Pat. No. 6,190,700).

A preferred composition for sustained bioavailability of a stress response polypeptide comprises a gene therapy construct comprising a gene therapy vectors, for example a gene therapy vector described herein below.

Viral Gene Therapy Vectors. Viral vectors of the invention are preferably disabled, e.g. replication-deficient. That is, they lack one or more functional genes required for their replication, which prevents their uncontrolled replication in vivo and avoids undesirable side effects of viral infection. Preferably, all of the viral genome is removed except for the minimum genomic elements required to package the viral genome incorporating the therapeutic gene into the viral coat or capsid. For example, it is desirable to delete all the viral genome except: (a) the Long Terminal Repeats (LTRs) or Invented Terminal Repeats (ITRs); and (b) a packaging signal. In the case of adenoviruses, deletions are typically made in the E1 region and optionally in one or more of the E2, E3 and/or E4 regions. Other viral vectors can be similarly deleted of genes required for replication. Deletion of sequences can be achieved by recombinant means, for example, involving digestion with appropriate restriction enzymes, followed by re-ligation. Replication-competent self-limiting or self-destructing viral vectors can also be used.

Nucleic acid constructs of the invention can be incorporated into viral genomes by any suitable means known in the art. Typically, such incorporation is performed by ligating the construct into an appropriate restriction site in the genome of the virus. Viral genomes can then be packaged into viral coats or capsids using any suitable procedure. In particular, any suitable packaging cell line can be used to generate viral vectors of the invention. These packaging lines complement the replication-deficient viral genomes of the invention, as they include, for example by incorporation into their genomes, the genes which have been deleted from the replication-deficient genome. Thus, the use of packaging lines allows viral vectors of the invention to be generated in culture.

Suitable packaging lines for retroviruses include derivatives of PA317 ce\dot{l}ls, ψ-2 cells, CRE cells, CRIP cells, E-86-GP cells, and 293GP cells. Line 293 cells are preferred for use with adenoviruses and adeno-associated viruses.

Plasmid Gene Therapy Vectors. A stress response protein free of an antigen binding domain can also be encoded by a plasmid. Advantages of a plasmid carrier include low toxicity and easy large-scale production. A polymer-coated plasmid can be delivered using electroporation as described by Fewell et al. (2001) Mol Ther 3:574-583. Alternatively, a plasmid can be combined with an additional carrier, for example a cationic polyamine, a dendrimer, or a lipid, that facilitates delivery. See e.g., Baher et al. (1999) Anticancer Res 19:2917-2924; Maruyama-Tabata et al. (2000) Gene Ther 7:53-60; and Tam et al. (2000) Gene Ther 7:1867-1874.

Liposomes. A stress response polypeptide of the present invention can also be delivered using a liposome. For example, a recombinantly produced stress response polypeptide can be encapsulated in liposomes. Liposomes can be prepared by any of a variety of techniques that are known in the art. See e.g., (1997). Current Protocols in Human Genetics on CD-ROM. John Wiley & Sons, New York; Lasic & Martin (1995) STEALTH® Liposomes. CRC Press, Boca Raton, Fla., United States of America; Janoff (1999) Liposomes: Rational Design. M. Dekker, New York; Gregoriadis (1993) Liposome Technology, 2nd ed. CRC Press, Boca Raton, Fla., United States of America; Betageri et al. (1993) Liposome Drug Delivery Systems. Technomic Pub., Lancaster; Pa., United States of America.; and U.S. Pat. Nos. 4,235,871; 4,551,482; 6,197,333; and 6,132,766. Temperature-sensitive liposomes can also be used, for example THERMOSOMES™ as disclosed in U.S. Pat. No. 6,200,598. Entrapment of a stress response polypeptide within liposomes of the present invention can be carried out using any conventional method in the art. In preparing liposome compositions, stabilizers such as antioxidants and other additives can be used.

Other lipid carriers can also be used in accordance with the claimed invention, such as lipid microparticles, micelles, lipid suspensions, and lipid emulsions. See e.g., Labat-Moleur et al. (1996) Gene Therapy 3:1010-1017; and U.S. Pat. Nos. 5,011,634; 6,056,938; 6,217,886; 5,948,767; and 6,210,707.

III.B. Targeting Ligands

As desired, a composition of the invention can include one or more ligands having affinity for a specific cellular marker to thereby enhance delivery of a stress response polypeptide to a site in need of treatment in a subject. Ligands include antibodies, cell surface markers, peptides, and the like, which act to home the stress response polypeptide to particular cells, for example tumor cells.

The terms “targeting” and “homing”, as used herein to describe the in vivo activity of a ligand following administration to a subject, each refer to the preferential movement and/or accumulation of a ligand in a target tissue (e.g., a tumor) as compared with a control tissue.

The term “target tissue” as used herein refers to an intended site for accumulation of a ligand following administration to a subject. For example, the methods of the present invention employ a target tissue comprising a tumor.

The term “control tissue” as used herein refers to a site suspected to substantially lack binding and/or accumulation of an administered ligand. For example, in accordance with the methods of the present invention, a non-cancerous tissue is a control tissue.

The terms “selective targeting” of “selective homing” as used herein each refer to a preferential localization of a ligand that results in an amount of ligand in a target tissue that is about 2-fold greater than an amount of ligand in a control tissue, more preferably an amount that is about 5-fold or greater, and most preferably an amount that is about 10-fold or greater. The terms “selective targeting” and “selective homing” also refer to binding or accumulation of a ligand in a target tissue concomitant with an absence of targeting to a control tissue, preferably the absence of targeting to all control tissues.

The terms “targeting ligand” and “targeting molecule” as used herein each refer to a ligand that displays targeting activity. Preferably, a targeting ligand displays selective targeting. Representative targeting ligands include peptides and antibodies.

The term “peptide” encompasses any of a variety of forms of peptide derivatives, that include amides, conjugates with proteins, cyclized peptides, polymerized peptides, conservatively substituted variants, analogs, fragments, peptoids, chemically modified peptides, and peptide mimetics. Representative peptide ligands that show tumor-binding activity include, for example, those described in U.S. Pat. Nos. 6,180,084 and 6,296,832.

The term “antibody” indicates an immunoglobulin protein, or functional portion thereof, including a polyclonal antibody, a monoclonal antibody, a chimeric antibody, a hybrid antibody, a single chain antibody (e.g., a single chain antibody represented in a phage library), a mutagenized antibody, a humanized antibody, and antibody fragments that comprise an antigen binding site (e.g., Fab and Fv antibody fragments). Representative antibody ligands that can be used in accordance with the methods of the present invention include antibodies that bind the tumor-specific antigens Her2/neu (v-erb-b2 avian erythroblastic leukemia viral oncogene homologue 2) (Kirpotin et al., 1997; Becerril et al., 1999) and antibodies that bind to CEA (carcinoembryonic antigen) (Ito et al., 1991). See also U.S. Pat. Nos. 5,111,867; 5,632,991; 5,849,877; 5,948,647; 6,054,561 and PCT International Publication No. WO 98/10795.

In an effort to identify ligands that are capable of targeting to multiple tumor types, targeting ligands have been developed that bind to target molecules present on tumor vasculature (Baillie et al., 1995; Pasqualini & Ruoslahti, 1996; Arap et al., 1998; Burg et al., 1999; Ellerby et al., 1999).

Antibodies, peptides, or other ligands can be coupled to drugs (e.g., a stress response polypeptide free of an antigen binding domain) or drug carriers using methods known in the art, including but not limited to carbodiimide conjugation, esterification, sodium periodate oxidation followed by reductive alkylation, and glutaraldehyde crosslinking. See e.g., Bauminger & Wilchek (1980) Methods Enzymol 70:151-159; Goldman et al. (1997) Cancer Res 57:1447-1451; Kirpotin et al. (1997) Biochemistry 36:66-75;—(1997). Current Protocols in Human Genetics on CD-ROM. John Wiley & Sons, New York; Neri et al. (1997) Nat Biotechnol 15:1271-1275; Park et al. (1997) Cancer Lett 118:153-160; and Pasqualini et al. (1997) Nat Biotechnol 15:542-546; U.S. Pat. No. 6,071,890; and European Patent No. 0 439 095. Alternatively, pseudotyping of a retrovirus can be used to target a virus towards a particular cell (Marin et al., 1997).

III.C. Formulation

A composition of the present invention preferably comprises a stress response polypeptide free of an antigen binding domain and a pharmaceutically acceptable carrier. Suitable formulations include aqueous and non-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some preferred ingredients are sodium dodecyl sulfate (SDS), for example in the range of 0.1 to 10 mg/ml, preferably about 2.0 mg/ml; and/or mannitol or another sugar, for example in the range of 10 to 100 mg/ml, preferably about 30 mg/ml; phosphate-buffered saline (PBS), and any other formulation agents conventional in the art.

The therapeutic regimens and pharmaceutical compositions of the invention can be used with additional adjuvants or biological response modifiers including, but not limited to, the cytokines interferon alpha (IFN-α), interferon gamma (IFN-γ), interleukin 2 (IL2), interleukin 4 (IL4), interleukin 6 (IL6), tumor necrosis factor (TNF), or other cytokine affecting immune cells.

III.D. Dose and Administration

Suitable methods for administration of a composition of the present invention include but are not limited to intravascular, subcutaneous, or intratumoral administration. For delivery of compositions to pulmonary pathways, compositions can be administered as an aerosol or coarse spray. A delivery method is selected based on considerations such as the type of the type of carrier or vector, therapeutic efficacy of the stress response polypeptide, and the condition to be treated. In a preferred embodiment of the invention, intravascular administration is employed.

Preferably, an effective amount of a composition of the invention is administered to a subject. For example, an “effective amount” is an amount of a composition comprising a stress response polypeptide free of an antigen binding domain sufficient to elicit an immune response. This is also referred to herein as an “immunostimulatory amount.” By way of additional example, an effective amount for tumor therapy comprises an amount sufficient to produce a measurable anti-tumor response (e.g., an anti-angiogenic response, a cytotoxic response, and/or tumor regression).

Actual dosage levels of active ingredients in a therapeutic composition of the invention can be varied so as to administer an amount of the active compound(s) that is effective to achieve the desired therapeutic response for a particular subject. The selected dosage level will depend upon a variety of factors including the activity of the therapeutic composition, formulation, the route of administration, combination with other drugs or treatments, the disease or disorder to be treated, and the physical condition and prior medical history of the subject being treated. Determination and adjustment of an effective amount or dose, as well as evaluation of when and how to make such adjustments, are known to those of ordinary skill in the art of medicine.

For local administration of viral vectors, previous clinical studies have demonstrated that up to 10¹³ pfu (plaque forming units) of virus can be injected with minimal toxicity. In human patients, 1×10⁹-1×10¹³ pfu are routinely used. See Habib et al. (1999) Hum Gene Ther 10:2019-2034. To determine an appropriate dose within this range, preliminary treatments can begin with 1×10⁹ pfu, and the dose level can be escalated in the absence of dose-limiting toxicity. Toxicity can be assessed using criteria set forth by the National Cancer Institute and is reasonably defined as any grade 4 toxicity or any grade 3 toxicity persisting more than 1 week. Dose is also modified to maximize anti-tumor and/or anti-angiogenic activity. Representative criteria and methods for assessing anti-tumor and/or anti-angiogenic activity are described herein below.

For soluble formulations of a stress response polypeptide of the present invention, conventional methods of extrapolating human dosage are based on doses administered to a murine animal model can be carried out using the conversion factor for converting the mouse dosage to human dosage: Dose Human per kg=Dose Mouse per kg×12 (Freireich et al., 1966). Drug doses are also given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich et al. (1966) Cancer Chemother Rep 50:219-244. Briefly, to express a mg/kg dose in any given species as the equivalent mg/m² dose, the dose is multiplied by the appropriate km factor. In adult humans, 100 mg/kg is equivalent to 100 mg/kg×37 kg/m²=3700 mg/m².

For the purposes of cell therapy, it is preferred to deliver cells, for example cells for ex vivo therapy, by intradermal or subcutaneous administration. A person of skill in the art will be able to choose an appropriate dosage, e.g. the number and concentration of cells, to take into account the fact that only a limited volume of fluid can be administered in this manner.

Additional dose techniques have been described in the art. See e.g., U.S. Pat. Nos. 5,326,902 and 5,234,933, and PCT International Publication No. WO 93/25521.

### EXAMPLES

The following Examples have been included to illustrate preferred modes of the invention. Certain aspects of the following Examples are described in terms of techniques and procedures found or contemplated by the present inventors to work well in the practice of the invention. These Examples are exemplified through the use of standard laboratory practices of the inventors. In light of the present disclosure and the general level of skill in the art, those of skill will appreciate that the following Examples are intended to be exemplary only and that numerous changes, modifications and alterations can be employed without departing from the spirit and scope of the invention.

### Example 1

### Preparation of GRP94ΔKDEL

In accordance with the present invention, this Example pertains to an alternative approach to biochemical purification of immunostimulatory stress response polypeptides. This approach employs secreted forms of GRP94 and GRP94 structural domains, as disclosed herein. GRP94 residence in the endoplasmic reticulum (ER) lumen is conferred by its C-terminal Lys-Asp-Glu-Leu (KDEL; SEQ ID NO:23) sequence (Munro & Pelham, 1987). Thus, a secretory form of GRP94 was engineered by deletion of its KDEL sequence to yield GRPΔKDEL.

Canine GRP94 cDNA was used as the template for all PCR reactions. For creation of GRP94ΔKDEL, the 5′ sense primer (SEQ ID NO:24) and the 3′ antisense primer (SEQ ID NO:25) were used to prepare a PCR product corresponding to the 5′ 2403 base pairs of the GRP94 coding region flanked by 5′ Sal I and 3′ Not I restriction sites. The PCR product was digested with Sal I/Not I then ligated into Sal I/Not I-digested pEF/myc/cyto vector (INVITROGEN™ Life Technologies of Carlsbad, Calif., United States of America). For creation of GRP94(1-337), the 5′ sense primer (SEQ ID NO:26) and the 3′ antisense primer (SEQ ID NO:27) were used to prepare a PCR product corresponding to the 5′ 1111 base pairs of the GRP94 coding region flanked by 5′ Sal I and 3′ Not I restriction sites. The PCR product was digested with Sal I/Not I then ligated into Sal I/Not I-digested pEF/myc/cyto vector. GRP94 NTD for recombinant expression was prepared using the 5′ sense primer (5′GGAATTCCATATGGACGATGAAGTCGATGTG3′) and the 3′antisense primer (5′CGGATCCTCAATTCATAAGCTCCCAATCCCA3′) to obtain a PCR product corresponding to bp 64-1,008 of the GRP94 coding sequence, flanked by 5′NdeI and 3′BamHI restriction sites. The PCR product was digested with NdeI/BamHI and ligated into NdeI/BamHI-digested pGEX vector (provided by D. Gewirth, Duke University Medical Center, Durham, N.C., United States of America). A preprolactin construct was also prepared to use as a control (Haynes et al., 1997).

### Example 2

### Expression of GRP94ΔKDEL in 4T1 Mammary Carcinoma Cells

A GRPΔKDEL cDNA construct, prepared as described in Example 1, was transfected into 4T1 mammary carcinoma cells. 4T1 cells (H-2d) and NIH-3T3 cells were cultured in Dulbecco's Modified Eagle's Medium (DMEM) supplemented with 10% fetal calf serum, 100 U/ml penicillin, and 100 μg/ml streptomycin. All cell lines were negative for mycoplasma DNA.

All transfections were performed using Lipofectamine™ reagent (Gibco BRL of Rockville, Md., United States of America) according to manufacturer's instructions. Mock transfections were performed with serum-free DMEM or with pEF/myc/cyto vector plus Lipofecatamine™ reagent. For dendritic cell (DC) maturation experiments, cells were transfected for 5 hours in serum-free DMEM plus DNA and Lipofectamine™ reagent. Cells were then rinsed gently with sterile phosphate buffered saline (PBS) and transferred to DC culture media. Conditioned media were collected for 72 hours, then subjected to low-speed centrifugation to clear cell debris. These media were then applied to day 6 dendritic cells, as described below.

To prepare transfected cells for fluorescence microscopy, cells were grown on glass coverslips in 6-well plates overnight to 50% confluence. Cells were then fixed in 4% paraformaldehyde in PBS for 10 minutes on ice. Fixed cells were permeabilized in 0.1% Triton X-100 in PBS for 15 minutes on ice. Blocking was performed by incubation in 1% bovine serum albumin (BSA) in PBS for 30 minutes at room temperature. Blocked cells were incubated in a 1:200 dilution of anti-myc antibody in 0.1% BSA in PBS for 1 hour at room temperature. Following extensive washing, cells were incubated in a 1:200 dilution of TEXAS RED® fluorescent dye (Molecular Probes, Inc. of Eugene, Wash., United States of America)-conjugated goat anti-mouse antibody conjugated (Cappel Laboratories of Westchester, Pa., United States of America) in 0.1% BSA in PBS for 1 hour at room temperature. Cells were again washed and mounted onto glass slides using mounting media (Difco Laboratories, Inc. of Detroit, Mich., United States of America). Fluorescently-labeled cells were visualized using a Zeiss LSM-410 scanning laser confocal microscope (Carl Zeiss Microimaging, Inc. of Thronwood, N.Y., United States of America). All images were processed using PHOTOSHOP® Version 6.0 software (Adobe Systems, Inc. of San Jose, Calif., United States of America).

Following transfection into 4T1 cells, GRPΔKDEL was distinguished from endogenous, full-length GRP94 through a myc epitope tag conferred by the expression vector. Anti-peptide antiserum against GRP94 (DU-120) was prepared according to the protocol of Harlow and Lane (Harlow & Lane, 1988), with antibody production being performed by Cocalico Biologicals of Reamstown, Pa., United States of America. Monoclonal antibody 9E10 to the myc epitope was purchased from Zymed Laboratories of South San Francisco, Calif., Unites States of America. Typically, a transfection efficiency of 25% was observed, with myc-positive cells displaying a canonical ER staining pattern. Transfection in the absence of plasmid DNA or in the presence of vector alone did not yield myc staining.

### Example 3

### Secretion and Processing of GRP94ΔKDEL by 4T1 Mammary Carcinoma Cells

To determine whether GRPΔKDEL was secreted, immunoprecipitations were performed on supernatants from GRPΔKDEL-transfected 4T1 cells and mock-transfected control cells. 4T1 cells were grown on glass coverslips, fixed, permeabilized, and incubated with anti-myc antibody (9E10). The myc tag was detected using a secondary antibody conjugated to TEXAS RED® fluorescent dye (Molecular Probes, Inc. of Eugene, Wash., United States of America).

Supernatants derived from transfected cells and immunoprecipitated with anti-myc antibody yielded a doublet of proteins of 100 and 110 kDa. Supernatants of mock-transfected cells yielded neither protein species. Similar patterns were observed in anti-myc immunoprecipitates of cell lysates, though as expected, immunoprecipitation with anti-GRP94 antibody yielded a prominent band in mock-transfected cells representing endogenous GRP94. Comparison of the relative mobilities of protein bands indicated that GRPΔKDEL has a slightly higher molecular weight than endogenous GRP94 due to the presence of the myc tag.

The appearance of GRPΔKDEL as a doublet can result from oligosaccharide modification during transit of the polypeptide through the Golgi apparatus. To explore this possibility, immunoprecipitates of chase media or cell lysates from GRPΔKDEL-transfected cells were subjected to digestion with endoglycosidase H (Endo H; available from Boehringer Mannheim of Indianapolis, Ind., United States of America) or peptide N-glycosidase F (PNGase-F; available from New England Biolabs of Beverly, Mass., United States of America) and separated by SDS-PAGE.

At 24 hours post-transfection or mock transfection, cells were starved by incubation in serum-, methionine-, and cysteine-free DMEM at 37° C. for 20 minutes. Pulse labeling was performed by incubation in serum-free, methionine-free, and cysteine-free DMEM supplemented with 100 μCi/ml ³⁵S-labeled Pro-Mix (Amersham Biosciences of Piscataway, N.J., United States of America) at 37° C. for 30 minutes. Cells were then washed and incubated in chase medium (growth medium plus 1 mM unlabeled L-methionine) at 37° C. for the indicated times. Samples of chase media were collected and cleared by centrifugation at 13,000 rpm for 5 minutes in a microfuge. Cells were lysed in ice-cold lysis buffer (150 mM NaCl, 50 mM Tris, pH 7.5, 0.05% SDS, 1% NP-40). Lysates were cleared of cell debris by centrifugation at 13,000 rpm for 5 minutes in a microfuge. All samples were pre-cleared with normal mouse serum and Pansorbin cells (Calbiochem of La Jolla, Calif., United States of America).

Proteins were immunoprecipitated from pre-cleared chase media and lysates using anti-GRP94 (DU-120) or anti-myc (9E10) antibodies and protein-A sepharose beads. Immunoprecipitates were processed for SDS-PAGE and resolved on 6%, 10%, or 12.5% polyacrylamide gels. Alternatively, immunoprecipitates were processed for glycosidase digestion as follows. Samples were incubated in denaturing buffer (0.5% SDS, 1% 2-mercaptoethanol) at 100° C. for 10 minutes.

For Endo H digestions, denatured proteins were incubated in G5 buffer (50 mM sodium citrate, pH 5.5) with or without 5 mU Endo H at 37° C. for 2.5 hours. For PNGase-F digestions, denatured proteins were incubated in G7 buffer (50 mM sodium phosphate, pH 7.5) plus 1% NP-40 with or without 0.8 mU PNGase-F at 37° C. for 2.5 hours. Samples were then processed for SDS-PAGE, resolved on 6% acrylamide gels. Radiolabeled proteins were visualized using a BASTM system for phoshpor imaging and MACBAS™-2.0 software (Fuji Medical Systems USA, Inc. of Stamford, Conn., United States of America).

In both chase media and cell lysates, the doublet resolved to a single protein species upon digestion with PNGase-F. Endogenous GRP94 in cell lysates shifted to a higher-mobility position upon PNGase-F digestion but remained distinct from GRPΔKDEL species. Endo H, an enzyme that cleaves high mannose oligosaccharides present on ER-resident proteins, did not affect the doublet present in chase media but resolved that present in cell lysates to a single species. These experiments showed that GRPΔKDEL is a single protein species, which undergoes heterogeneous oligosaccharide modification along the exocytic pathway.

### Example 4

### GRPΔKDEL Secretion Kinetics

Deletion of the KDEL retention/retrieval sequence of ER resident lumenal proteins allowed secretion of GRPΔKDEL, albeit often at markedly slower rates than that observed in bona fide secretory proteins.

To assess the relative rate of GRPΔKDEL secretion, pulse-chase studies were performed on 4T1 cells that had been transfected with constructs encoding either GRPΔKDEL or the secretory hormone preprolactin. 4T1 breast carcinoma cells were metabolically labeled for 30 minutes. Following initiation of the chase period, cell and media samples were collected, and GRPDKDEL or prolactin were recovered by immunoprecipitation and the GRP94 treated with PNGase-F. Proteins were resolved by SDS-PAGE on 6% gels for GRPΔKDEL or 10% gels for prolactin. Protein bands were analyzed using a BASTM system for phoshpor imaging and MACBAS™-2.0 software (Fuji Medical Systems USA, Inc. of Stamford, Conn., United States of America). An amount of protein quantified in each band was used to determine the percent total GRPΔKDEL or prolactin present in the media or cell lysate at each time point.

These experiments indicated that GRPΔKDEL secretion is efficient, with a half-time of 120 minutes versus 60 minutes for native prolactin. Interestingly, endogenous GRP94, was seen as a distinct band in immunoprecipitates of cell lysates, and remained at fairly constant levels over time, indicating that heterodimerization of full-length GRP94 with GRPΔKDEL was not a significant competing assembly reaction.

### Example 5

### GRPΔKDEL Secreted from 4T1 Mammary Carcinoma Cells or NIH3T3 Fibroblasts Protects Against 4T1 Tumor Challenge

To assess the importance of antigen-independent effects in GRP94-mediated tumor rejection, a 4T1 murine tumor progression model was studied. 4T1 mammary carcinoma cells were chosen as a model tumor cell line because they are highly aggressive, metastasize widely, and respond poorly to therapy (Coveney et al., 1996; Lohr et al., 2001). To ensure that cells used in the immunization phase did not establish tumors, cells were irradiated prior to injection into animals. Irradiation did not affect levels of GRPΔKDEL expression or secretion (FIG. 1A).

Transfected 4T1 and NIH3T3 (H-2q) cells (American Type Culture Collection of Manassas, Va., United States of America) were prepared as described in Example 2. Cells were irradiated (10,000 rad) at 24 hours post-transfection.

Female BALB/c mice (H-2d) were obtained from Charles River Laboratories (Raleigh, N.C., United States of America). Female C57BU6 mice (H-2^{b}) were obtained from NCI Frederick Cancer Research and Development Center (Frederick, Md., United States of America). Animals were maintained and treated in accordance with all applicable guidelines of the Institutional Animal Care and Use Committee (IACUC) of the American Association for Laboratory Animal Science.

Transfected, irradiated cells were washed extensively with sterile PBS, then injected into the left hind limb skin of BALB/c mice at 2-4×10⁶ cells per animal. Immunizations were given weekly for four consecutive weeks. At week 5, mice were challenged with 1×10⁶ 4T1 cells in sterile PBS by injection into the skin of the right back. Tumor length, width, and height were measured every 2-3 days following challenge, and tumor volume was calculated using the following formula:

Volume=(π/6)×length×width×height

At the completion of the study, animals were sacrificed, and lungs were resected and weighed. For tumor volume and lung weight data, the significance of differences between groups was analyzed with the Wilcoxon rank sum test.

In one set of studies, GRPΔKDEL-transfected or mock-transfected 4T1 cells were used in the vaccination phase prior to challenge with live 4T1 cells. As expected, both control mice vaccinated with PBS and mice vaccinated with mock-transfected 4T1 cells (4T1-mock) displayed rapid tumor progression (FIGS. 1B, 1C, and 1E). Mock-transfected 4T1 cells provided a modest induction of anti-tumor immune responses compared to PBS, but the difference in tumor volumes between these two groups was not statistically significant (p=0.33). Notably, mice vaccinated with GRPΔKDEL-secreting 4T1 cells (4T1-ΔKDEL) displayed markedly delayed tumor progression compared to control animals (FIGS. 1D-1E). The difference in tumor volumes between this group and control groups was statistically significant (p=0.00005 for PBS versus 4T1-ΔKDEL, and p=0.0021 for 4T1-mock versus 4T1-ΔKDEL).

In a second study, GRPΔKDEL-transfected or mock-transfected NIH-3T3 fibroblasts were used in the vaccination phase preceding challenge with 4T1 cells. Again, both control mice vaccinated with PBS and mice vaccinated with mock-transfected NIH-3T3 cells (NIH-mock) displayed rapid tumor progression (FIGS. 1B, 1F, and 1H). The difference in tumor volumes between these groups was not statistically significant (p=0.57). Interestingly, animals that were immunized with GRPΔKDEL-secreting NIH-3T3 cells (NIH-ΔKDEL) displayed markedly delayed tumor progression (FIGS. 1G-1H; p=0.0013 for PBS versus NIH-ΔKDEL, and p=0.0022 for NIH-mock versus NIH-ΔKDEL).

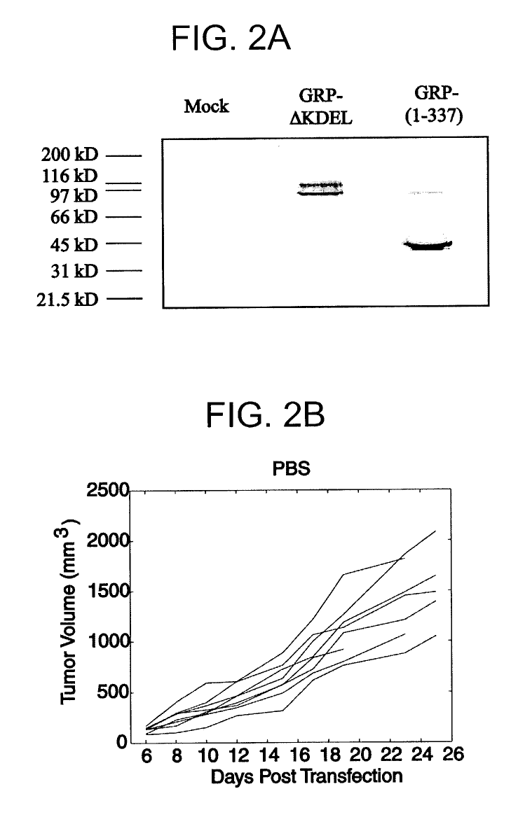

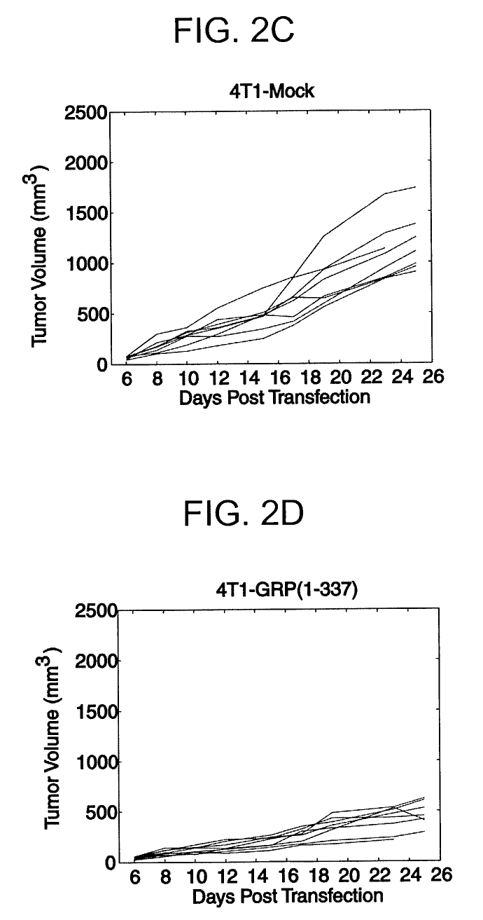

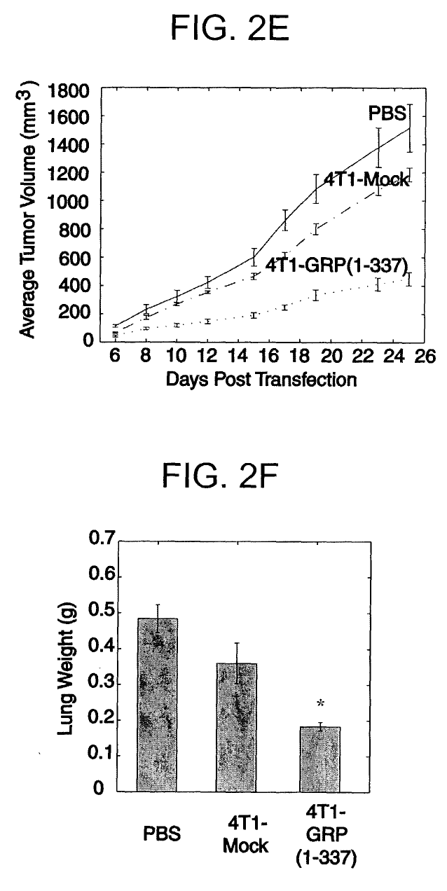

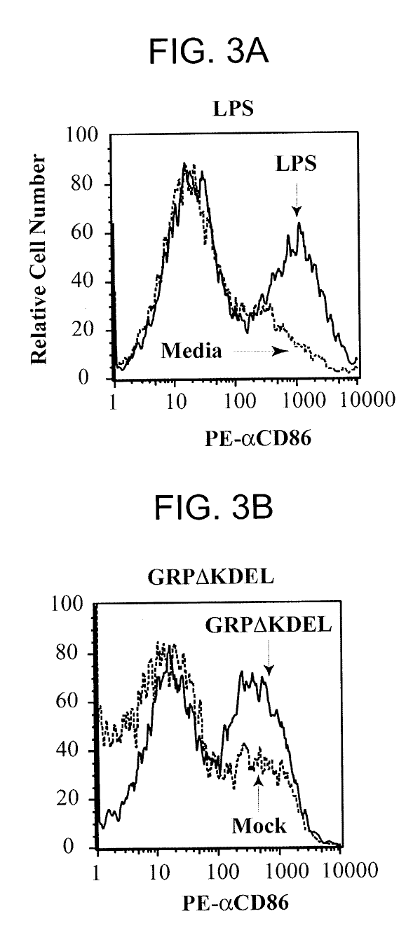

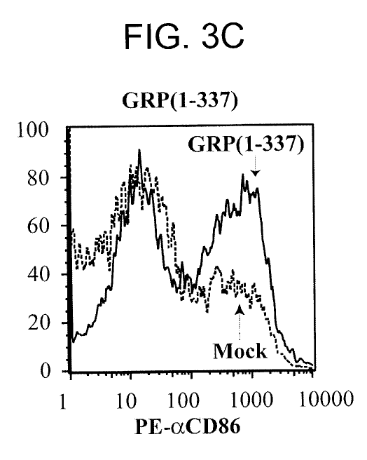

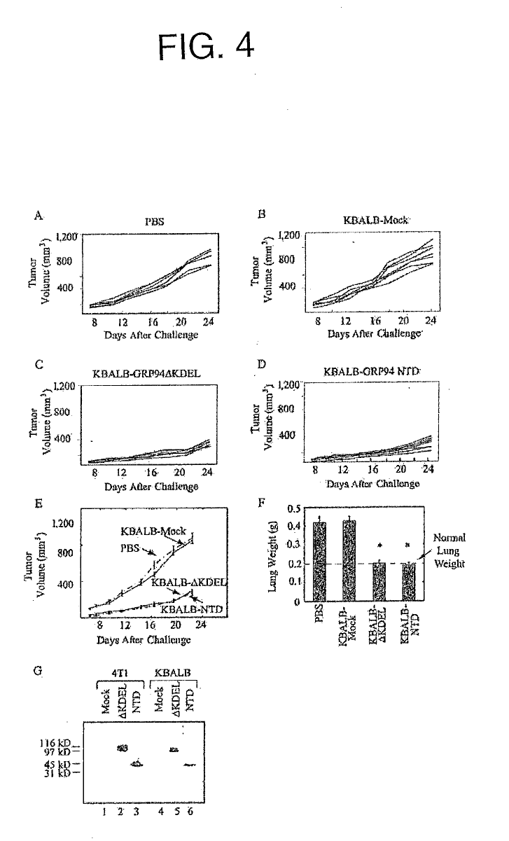

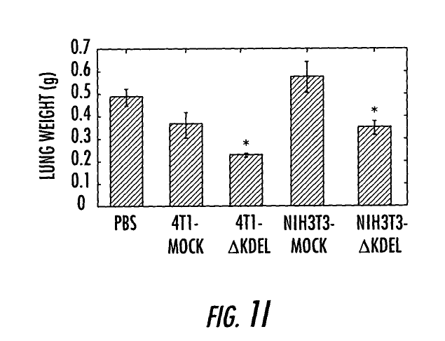

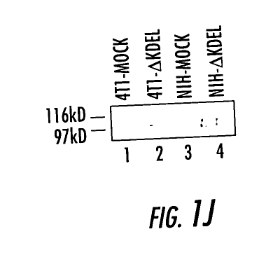

Following sacrifice, lungs were excised from animals in each group and weighed as a measure of tumor metastasis. Lungs from animals vaccinated with GRPΔKDEL-secreting 4T1 cells weighed significantly less than those of control animals (FIG. 11; p=0.0012 for PBS versus 4T1-ΔKDEL, and p=0.010 for 4T1-mock vs. 4T1-ΔKDEL). The lungs of animals vaccinated with GRPΔKDEL-secreting NIH3T3 cells also weighed significantly less than those of control mice (FIG. 11; p=0.025 for PBS-vaccinated versus NIH-ΔKDEL, and p=0.026 for NIH-mock versus NIH-ΔKDEL). Animals receiving immunizations of mock-transfected 4T1 cells demonstrated slightly reduced lung weights compared to PBS-vaccinated controls, though this difference was not statistically significant (p=0.07). These data demonstrate that secretion of GRP94 by irradiated tumor cells provides a significant suppression of tumor growth and metastatic progression. Further, these data were unexpected, as they indicate that the tissue source of GRP94 was not an essential determinant in the induction of GRP94-dependent suppression of tumor growth and metastatic progression.

To compare the relative levels of GRPΔKDEL secretion by 4T1 and NIH-3T3 cells, pulse-chase experiments were performed (FIG. 1J). The level of GRPΔKDEL secretion by both cell types was comparable, indicating that the tumor suppression observed after immunization with GRP94-secreting fibroblasts does not result from an increased GRP94 dose as compared with GRP94-secreting 4T1 cells.

### Example 6

### The Amino-Terminal Regulatory Domain of GRP94 Protects Against Tumor Challenge

The observation that GRP94 secreted from NIH3T3 cells protected against 4T1 tumor challenge suggested that antigen-independent mechanisms play an important role in GRP94-mediated tumor rejection. Alternatively, 4T1 and NIH-3T3 cell lines shared common, immunodominant antigens that were responsible for the observed results. To distinguish between these explanations, a form of GRP94 that lacked the ability to bind peptides but retained the ability to directly activate immune responses was prepared.

The peptide-binding site of GRP94 has been identified previously to reside in the C-terminal region of the molecule (Linderoth et al., 2000). To create a non-peptide binding GRP94 polypeptide, a construct was prepared to encode the amino-terminal regulatory domain of GRP94, corresponding to amino acids 1-337 of the protein, GRP(1-337) (SEQ ID NO:2). This region of GRP94 comprises a discrete structural domain that serves as the binding site for anti-tumor compounds and adenosine nucleotides (Prodromou et al., 1997b; Prodromou et al., 1997a; Stebbins et al., 1997; Rosser & Nicchitta, 2000). Importantly, no structural motifs exist in this domain that could function in the binding of peptides of suitable length for assembly onto MHC class I molecules (≧9 amino acids). See Stebbins et al. (1997) Cell 89:239-250. Upon transfection of GRP(1-337 cDNA into 4T1 cells, a 36 kDa protein was expressed and recognized by a polyclonal antibody raised against the N-terminal domain of GRP94. GRP94(1-337) appeared as a single species in anti-GRP94 immunoprecipitations, indicating it did not undergo the extensive heterogeneous glycosylation observed for GRPΔKDEL.

In vivo tumor rejection studies were performed using 4T1 cells transfected with GRP(1-337) in the vaccination phase (FIGS. 2A-2D). Mice receiving immunizations of GRP(1-337)-transfected 4T1 cells displayed substantially smaller tumor size and overall slower tumor growth rates as compared with mice vaccinated with PBS or mock-transfected cells (p=0.0002 for PBS versus 4T1-GRP(1-337), and p=0.0006 for 4T1-mock versus 4T1-GRP(1-337)).

At the time of sacrifice, lungs were excised from animals in all groups and weighed (FIG. 2D). Animals vaccinated with GRP(1-337)-secreting 4T1 cells displayed lung weights that were significantly lower than those of control animals (p=0.0031 for PBS versus 4T1-GRP(1-337) and p=0.0008 for 4T1-mock versus 4T1-GRP(1-337)). These observations demonstrated that the amino-terminal domain of GRP94 was effective in protecting against subsequent 4T1 tumor challenge and that antigen-independent mechanisms play an important role in the immunomodulatory activities of GRP94.

### Example 7

GRP94ΔKDEL and GRP94(1-337) Elicit Dendritic Cell Maturation

Bone marrow-derived dendritic cells (DCs) were propagated from bone marrow progenitor cells according to the method of Inaba et al. (1992) J Exp Med 176:1693-1702 with minor modifications. Bone marrow precursors were flushed from the tibiae and femurs of C57BL/6 mice and plated at 1×10⁶ cells/ml in DC culture media (RPMI 1640 plus 5% heat-inactivated fetal calf serum, 100 U/ml penicillin, 100 μg/ml streptomycin, 20 μg/ml gentamicin, 50 μM 2-mercaptoethanol) supplemented with granulocyte macrophage-colony stimulating factor (GM-CSF; 5% culture supernatant from X63 cells stably transfected with murine GM-CSF cDNA). Cultures were washed on day 2 and day 4.

For maturation assays, day 6 DCs were harvested, pelleted by brief centrifugation, and transferred to fresh 6-well plates at 5×10⁵ cells/ml after resuspension in the appropriate control media or conditioned media. For DC maturation studies, cells were harvested on day 7, and Fc receptors blocked with immunoglobulin prior to staining with Phycoerythrin (PE)-conjugated rat anti-mouse CD86 antibody (BD PharMingen of San Diego, Calif., United States of America). Following fixation, cells were then analyzed by flow cytometry using FACSCAN™ software (Becton, Dickinson & Company of Franklin Lakes, N.J., United States of America) and CELLQUEST™ software (Becton, Dickinson & Company of Franklin Lakes, N.J., United States of America).

Exposure of immature dendritic cells to GRP94 results in upregulation of major histocompatibility class I and class II, expression of co-stimulatory molecules such as B7-2 (CD86), and secretion of cytokines (Basu et al., 2000; Binder et al., 2000b; Singh-Jasuja et al., 2000a). To test the ability of a non-peptide binding stress response polypeptide to modulate immune responses, the ability of secreted GRPΔKDEL and GRP(1-337) to elicit dendritic cell maturation was assayed in vitro.

Dendritic cells isolated on day 6 of culture typically display an immature phenotype characterized by expression of CD11 c (CD11c⁺), intermediate levels of MHC Class II polypeptides (MHC Class II^{intermediate}), lack of GR-1 expression (GR-1⁻), low levels of CD80 polypeptides (CD80^{low}), and low levels of CD86 polypeptides (CD86^{low}). See Inaba et al. (1992) J Exp Med 176:1693-1702.

Upon exposure to a stimulatory molecule such as lipopolysaccharride (LPS), dendritic cells convert to a mature phenotype characterized by expression of CD11c (CD11c⁺), high levels of MHC Class II polypeptides (MHC Class II^{high}), lack of GR-1 expression (GR-1⁻), high levels of CD80 polypeptides (CD80^{high}), and high levels of and CD86 polypeptides (CD₈₆^{high}). See Brinker et al. (2001) Am J Physiol Lung Cell Mol Physiol 281:L1453-1463.

GRP94 was chosen as a marker to monitor the DC response to GRPΔKDEL and GRP(1-337) based on its ability to upregulate CD86 expression on dendritic cells (Basu et al., 2000; Singh-Jasuja et al., 2000a). As expected, incubation of dendritic cells in GM-CSF-free media resulted in the majority of cells expressing low levels of CD86 (FIG. 3A). In contrast, incubation in LPS-containing media produced a robust upregulation of cell-surface CD86 (FIG. 3A). Compared to cells incubated in media alone, DCs exposed to conditioned media from mock-transfected, GRPΔKDEL-transfected, or GRP(1-337)-transfected 4T1 cells displayed an upregulation of CD86 expression. The level of CD86 observed following exposure of dendritic cells to GRPΔKDEL- and GRP(1-337)-transfected 4T1 supernatants was higher than a level observed following exposure of dendritic cells to mock-transfected 4T1 supernatant. The ability of conditioned media from mock-transfected 4T1 cells to mature DCs indicates that this cell type likely secretes factors other than GRP94 that are capable of eliciting this response. Incubation of immature DCs in conditioned media from mock-transfected NIH3T3 cells, on the other hand, produced little upregulation of CD86 expression compared to media alone (FIGS. 3B-3C). Notably, conditioned media from GRPΔKDEL-transfected or GRP (1-337)-transfected NIH-3T3 cells yielded a robust upregulation of CD86 (FIGS. 3B-3C). These data indicate that both secreted GRP94 and its amino-terminal domain are able to elicit dendritic cell maturation regardless of cell type of origin.

### Example 8

### Interaction of GRP94 NTD with APC

The interaction of GRP94 NTD with APC was also examined. GRP94 NTD displayed cell surface binding to bone marrow-derived DCs, elicited peritoneal macrophages, and the macrophage-derived cell line RAW264.7. Little or no binding of GRP94 NTD was observed in B16-F10 melanoma cells, COS7 kidney cells, or NIH-3T3 fibroblasts. Fluorescently labeled full-length GRP94 similarly displayed binding to DCs, peritoneal macrophages, and RAW264.7 cells with little to no binding to B16-F10, COS7, or NIH-3T3 cells.

As a result of cell surface binding to APCs, GRP94 undergoes receptor-mediated endocytosis. To investigate the fate of cell surface-bound GRP94 NTD, fluorescently labeled GRP94 or GRP94 NTD was first bound to elicited peritoneal macrophages at 40° C. After binding, unbound protein was removed by washing and the cells were warmed to 37° C. In cells fixed before warming, prominent cell surface binding of both GRP94 and the GRP94 NH2-terminal domain was observed (0 minutes). After 10 minutes at 37° C., both GRP94 and GRP94 NH2-terminal domain gained entry to the cell as indicated by a punctate intracellular peri-plasmalemmal staining pattern (10 minutes). At longer incubation intervals, GRP94 and GRP94 NH2-terminal domain were more widely dispersed throughout the cell interior in prominent vesicular structures. At each time point, full-length GRP94 co-localized with the GRP94 NH2-terminal domain. The internalization of GRP94 and GRP94 NH2-terminal domain was not interdependent. Both proteins were internalized and displayed a similar trafficking pattern in the absence of the other. These observations indicate that the NH2-terminal domain of GRP94 displays the pattern elements necessary for recognition and clearance by APCs.

### Example 9

### Vaccination Trials

Vaccination trials were performed with haplotype-matched KBALB fibroblasts transfected with GRPΔKDEL or GRP94 NTD cDNA (transfections performed substantially as disclosed herein above, see e.g. Example 5). The results of these studies are depicted in FIGS. 4A-4G, where it was observed that animals immunized with GRP94 NTD secreting KBALB cells displayed reduced primary tumor burden than animals immunized with PBS or mock-transfected cells (P≦0.0003 for PBS vs. KBALB-GRPΔKDEL, P≦0.0003 for PBS vs. KBALB-GRP94 NTD, and P≦0.24 for PBS vs. KBALB-Mock; FIGS. 4A-4E). In addition, animals immunized with syngeneic fibroblasts secreting GRPΔKDEL or GRP94 NTD had decreased metastatic tumor burden (P≦0.0003 for PBS vs. KBALB-GRPΔKDEL, P≦0.0002 for PBS vs. KBALB-GRP94 NTD, and P≦0.8 for PBS vs. KBALB-Mock; FIG. 4F). Together, these observations demonstrate that the NH2-terminal domain of GRP94 recapitulates the activity of GRPΔKDEL in suppressing tumor growth and metastatic progression.

To compare the relative levels of GRPΔKDEL and GRP94 NTD secretion by 4T1 and KBALB cells, pulse chase experiments were performed (FIG. 4G). The level of GRPΔKDEL and GRP94 NTD secretion by both cell types was comparable, indicating that the tumor suppression observed after immunization did not reflect differences in GRP94 dose.

### Example 10

### Tumor Histology

To gain insight into variations in the tumor microenvironment among the vaccination groups in the immunization and challenge protocols described above, tumors from the control and experimental groups were excised at the time of sacrifice, fixed, and prepared for histological analysis. In all cases, 4T1 tumors were characterized by the predominance of malignant-appearing cells with hyperchromatic nuclei and high nuclear to cytoplasmic ratios. Mitotic figures were abundant and several a typical mitoses were observed, although the mitotic rate did not differ significantly among the various vaccination groups. The tumors featured large tracts of necrosis with obvious pyknosis and karyolysis of nuclear material. At the midpoint of the study, tumors were characterized by the presence of macrophages, neutrophils, and rare lymphocytes, although the relative number of inflammatory cells did not differ greatly among the various vaccination groups. As seen at low power, tumors in control animals receiving vaccinations of PBS, mock-transfected 4T1 cells or mock-transfected NIH-3T3 cells were larger in size and contained larger areas of necrosis than tumors in animals receiving vaccinations of GRPΔKDEL of GRP94 NTD transfected 4T1 or NIH-3T3 cells.

### REFERENCES

The references listed below as well as all references cited in the specification are incorporated herein by reference to the extent that they supplement, explain, provide a background for or teach methodology, techniques and/or compositions employed herein.—

(1997) Current Protocols in Human Genetics on CD-ROM. New York: John Wiley & Sons.

Altschul S F, Gish W, Miller W, Myers E W & Lipman D J (1990) Basic Local Alignment Search Tool. J Mol Biol 215:403-410.

Amato R, Murray L, Wood L, Savary C, Tomasovic S & Reitsma D (1999) Active Specific Immunotherapy in Patients with Renal Cell Carcinoma (RCC) Using Autologous Tumor Derived Heat Shock Protein-Peptide Complex-96 (HSPP-96) Vaccine. ASCO Meeting abstract.

Amato R, Murray L, Wood L, Savary C, Tomasovic S & Reitsma D (2000) Active Specific Immunotherapy in Patients with Renal Cell Carcinoma (RCC) Using Autologous Tumor Derived Heat Shock Protein-Peptide Complex-96 (HSPP-96) Vaccine. ASCO Meeting abstract.

Arap W, Pasqualini R & Ruoslahti E (1998) Cancer Treatment by Targeted Drug Delivery to Tumor Vasculature in a Mouse Model. Science 279:377-380.

Arnold D, Faath S, Rammensee H & Schild H (1995) Cross-Priming of Minor Histocompatibility Antigen-Specific Cytotoxic T Cells Upon Immunization with the Heat Shock Protein GP96. J Exp Med 182:885-889.

Arnold D, Wahl C, Faath S, Rammensee H G & Schild H (1997) Influences of Transporter Associated with Antigen Processing (TAP) on the Repertoire of Peptides Associated with the Endoplasmic Reticulum-Resident Stress Protein GP96. J Exp Med 186:461-466.

Arnold-Schild D, Hanau D, Spehner D, Schmid C, Rammensee H G, de la Salle H & Schild H (1999) Cutting Edge: Receptor-Mediated Endocytosis of Heat Shock Proteins by Professional Antigen-Presenting Cells. J Immunol 162:3757-3760.

Asea A, Kabingu E, Stevenson MA & Calderwood SK (2000) Hsp70 Peptide-bearing and Peptide-Negative Preparations Act as Chaperokines. Cell Stress Chaperones 5:425-431.

Asea A, Kraeft S K, Kurt-Jones E A, Stevenson M A, Chen L B, Finberg R W, Koo G C & Calderwood S K (2000) Hsp70 Stimulates Cytokine Production through a CD14-Dependant Pathway, Demonstrating Its Dual Role as a Chaperone and Cytokine. Nat Med 6:435-442.

Ausubel F, ed (1995) Short Protocols in Molecular Biology, 3rd ed. Wiley, New York.

Baher A G, Andres M L, Folz-Holbeck J, Cao J D & Gridley D S (1999) A Model Using Radiation and Plasmid-Mediated Tumor Necrosis Factor-Alpha Gene Therapy for Treatment of Glioblastomas. Anticancer Res 19:2917-2924.

Baillie C T, Winslet M C & Bradley N J (1995) Tumour Vasculature—a Potential Therapeutic Target. Br J Cancer 72:257-267.

Barton G J (1998) Protein Sequence Alignment Techniques. Acta Crystallogr D Biol Crystallogr 54:1139-1146.

Basu S & Srivastava PK (2000) Heat Shock Proteins: The Fountainhead of Innate and Adaptive Immune Responses. Cell Stress Chaperones 5:443-451.

Basu S, Binder R J, Ramalingam T & Srivastava P K (2001) Cd91 Is a Common Receptor for Heat Shock Proteins Gp96, Hsp90, Hsp70, and Calreticulin. Immunity 14:303-313.

Basu S, Binder R J, Suto R, Anderson K M & Srivastava P K (2000) Necrotic but Not Apoptotic Cell Death Releases Heat Shock Proteins, Which Deliver a Partial Maturation Signal to Dendritic Cells and Activate the NF-Kappa B Pathway. Int Immunol 12:1539-1546.

Batzer M A, Carlton J E & Deininger P L (1991) Enhanced Evolutionary PCR Using Oligonucleotides with Inosine at the 3′-Terminus. Nucleic Acids Res 19:5081.

Bauminger S & Wilchek M (1980) The Use of Carbodiimides in the Preparation of Immunizing Conjugates. Methods Enzymol 70:151-159.

Becerril B, Poul M A & Marks J D (1999) Toward Selection of Internalizing Antibodies from Phage Libraries. Biochem Biophys Res Commun 255:386-393.

Betageri G V, Jenkins S A & Parsons D L (1993) Liposome Drug Delivery Systems. Technomic Pub., Lancaster, Pa., United States of America.

Binder R J, Han D K & Srivastava P K (2000a) CD91: A Receptor for Heat Shock Protein GP96. Nat Immunol 1:151-155.

Binder R J, Anderson K M, Basu S & Srivastava P K (2000b) Cutting Edge: Heat Shock Protein GP96 Induces Maturation and Migration of CD11c+ Cells in Vivo. J Immunol 165:6029-6035.

Blachere N E & Srivastava P K (1995) Heat Shock Protein-Based Cancer Vaccines and Related Thoughts on Immunogenicity of Human Tumors. Semin Cancer Biol 6:349-355.

Blachere N E, Li Z, Chandawarkar R Y, Suto R, Jaikaria N S, Basu S, Udono H & Srivastava P K (1997) Heat Shock Protein-Peptide Complexes, Reconstituted in Vitro, Elicit Peptide-Specific Cytotoxic T Lymphocyte Response and Tumor Immunity. J Exp Med 186:1315-1322.

Botzler C, Issels R & Multhoff G (1996a) Heat-Shock Protein 72 Cell-Surface Expression on Human Lung Carcinoma Cells in Associated with an Increased Sensitivity to Lysis Mediated by Adherent Natural Killer Cells. Cancer Immunol Immunother 43:226-230.

Botzler C, Kolb H J, Issels R D & Multhoff G (1996b) Noncytotoxic Alkyl-Lysophospholipid Treatment Increases Sensitivity of Leukemic K562 Cells to Lysis by Natural Killer (NK) Cells. Int J Cancer 65:633-638.

Breloer M, Marti T, Fleischer B & von Bonin A (1998) Isolation of Processed, H-2 kb-Binding Ovalbumin-Derived Peptides Associated with the Stress Proteins Hsp70 and Gp96. Eur J Immunol 28:1016-1021.

Brinker K G, Martin E, Borron P, Mostaghel E, Doyle C, Harding C V & Wright J R (2001) Surfactant Protein D Enhances Bacterial Antigen Presentation by Bone Marrow-Derived Dendritic Cells. Am J Physiol Lung Cell Mol Physiol 281 L1453-1463.

Bumol T F, Marder P, DeHerdt S V, Borowitz M J & Apelgren L D (1988) Characterization of the Human Tumor and Normal Tissue Reactivity of the KS 1/4 Monoclonal Antibody. Hybridoma 7:407-415.

Burg M A, Pasqualini R, Arap W, Ruoslahti E & Stallcup W B (1999) Ng2 Proteoglycan-Binding Peptides Target Tumor Neovasculature. Cancer Res 59:2869-2874.

Castellino F, Boucher P E, Eichelberg K, Mayhew M, Rothman J E, Houghton A N & Germain R N (2000) Receptor-Mediated Uptake of Antigen/Heat Shock Protein Complexes Results in Major Histocompatibility Complex Class I Antigen Presentation Via Two Distinct Processing Pathways. J Exp Med 191:1957-1964.

Chapman J R (2000) Mass Spectrometry of Protein and Peptides. Humana Press, Totowa, N.J., United States of America.

Chappell T G, Konforti B B, Schmid S L & Rothman J E (1987) The Atpase Core of a Clathrin Uncoating Protein. J Biol Chem 262:746-751.

Chen W, Syldath U, Bellmann K, Burkart V & Kolb H (1999) Human 60-kDa Heat-Shock Protein: A Danger Signal to the Innate Immune System. J Immunol 162:3212-3219.

Coveney E, Clary B, lacobucci M, Philip R & Lyerly K (1996) Active Immunotherapy with Transiently Transfected Cytokine-Secreting Tumor Cells Inhibits Breast Cancer Metastases in Tumor-Bearing Animals. Surgery 120:265-272; discussion 272-263.

Csermely P, Schnaider T, Soti C, Prohaszka Z & Nardai G (1998) The 90-kDa Molecular Chaperone Family: Structure, Function, and Clinical Applications. A Comprehensive Review. Pharmacol Ther 79:129-168.

Demotz S, Grey H M, Appella E & Sette A (1989) Characterization of a Naturally Processed Mhc Class Ii-Restricted T-Cell Determinant of Hen Egg Lysozyme. Nature 342:682-684.

Easton D P, Kaneko Y & Subjeck J R (2000) The Hsp110 and GRP170 Stress Proteins: Newly Recognized Relatives of the Hsp70s. Cell Stress Chaperones 5:276-290.

El-Assal O N, Yamanoi A, Soda Y, Yamaguchi M, Igarashi M, Yamamoto A, Nabika T & Nagasue N (1998) Clinical Significance of Microvessel Density and Vascular Endothelial Growth Factor Expression in Hepatocellular Carcinoma and Surrounding Liver: Possible Involvement of Vascular Endothelial Growth Factor in the Angiogenesis of Cirrhotic Liver. Hepatology 27:1554-1562.

Ellerby H M, Arap W, Ellerby L M, Kain R, Andrusiak R, Rio G D, Krajewski S, Lombardo C R, Rao R, Ruoslahti E, Bredesen D E & Pasqualini R (1999) Anti-Cancer Activity of Targeted Pro-Apoptotic Peptides. Nat Med 5:1032-1038.

Elliott T, Townsend A & Cerundolo V (1990) Antigen Presentation. Naturally Processed Peptides. Nature 348:195-197.

Estin CD, Stevenson U, Kahn M, Hellstrom I & Hellstrom K E (1989) Transfected Mouse Melanoma Lines That Express Various Levels of Human Melanoma-Associated Antigen P97. J Natl Cancer Inst 81:445-448.

Eton O, East M, Ross M, Savary C, Tomasovic S, Reitsma D, Hawkins E & Srivastava P (2000) Autologous Tumor-Derived Heat Shock Protein Complex-96 (HSPPc-96) in Patients(Pts) with Metastatic Melanoma. Proc Am Assoc Canc Res 41:543.

European Patent No. 0 439 095

Falk K, Rotzschke O & Rammensee H G (1990) Cellular Peptide Composition Governed by Major Histocompatibility Complex Class I Molecules. Nature 348:248-251.

Falk K, Rotzschke O, Stevanovic S, Jung G & Rammensee H G (1991) Allele-Specific Motifs Revealed by Sequencing of Self-Peptides Eluted from Mhc Molecules. Nature 351:290-296.

Fearon D T (1997) Seeking Wisdom in Innate Immunity. Nature 388:323-324.

Fearon D T & Locksley R M (1996) The Instructive Role of Innate Immunity in the Acquired Immune Response. Science 272:50-53.

Feder M E & Hofmann G E (1999) Heat-Shock Proteins, Molecular Chaperones, and the Stress Response: Evolutionary and Ecological Physiology. Annu Rev Physiol 61:243-282.

Fewell J G, MacLaughlin F, Mehta V, Gondo M, Nicol F, Wilson E & Smith L C (2001) Gene Therapy for the Treatment of Hemophilia B Using PINC-Formulated Plasmid Delivered to Muscle with Electroporation. Mol Ther 3:574-583.

Fisher F W & Cook N B (1998) Fundamentals of Diagnostic Mycology. W.B. Saunders, Philadelphia.

Freireich E J, Gehan E A, Rall D P, Schmidt L H & Skipper H E (1966) Quantitative Comparison of Toxicity of Anticancer Agents in Mouse, Rat, Hamster, Dog, Monkey, and Man. Cancer Chemother Rep 50:219-244.

Glover D M & Hames B D (1995) DNA Cloning: A Practical Approach, 2nd ed. IRL Press at Oxford University Press, Oxford; N.Y.

Goldman C K, Rogers B E, Douglas J T, Sosnowski B A, Ying W, Siegal G P, Baird A, Campain J A & Curiel D T (1997) Targeted Gene Delivery to Kaposi's Sarcoma Cells Via the Fibroblast Growth Factor Receptor. Cancer Res 57:1447-1451.

Gossen M & Bujard H (1992) Tight Control of Gene Expression in Mammalian Cells by Tetracycline-Responsive Promoters. Proc Natl Acad Sci USA 89:5547-5551.

Gossen M & Bujard H (1993) Anhydrotetracycline, a Novel Effector for Tetracycline Controlled Gene Expression Systems in Eukaryotic Cells. Nucleic Acids Res 21:4411-4412.

Gossen M, Freundlieb S, Bender G, Muller G, Hillen W & Bujard H (1995) Transcriptional Activation by Tetracyclines in Mammalian Cells. Science 268:1766-1769.

Gragerov A, Zeng L, Zhao X, Burkholder W & Gottesman M E (1994) Specificity of Dnak-Peptide Binding. J Mol Biol 235:848-854.

Gregoriadis G (1993) Liposome Technology, 2nd ed. CRC Press, Boca Raton, Fla., United States of America.

Habib N A, Hodgson H J, Lemoine N & Pignatelli M (1999) A Phase I/Ii Study of Hepatic Artery Infusion with Wtp53-Cmv-Ad in Metastatic Malignant Liver Tumours. Hum Gene Ther 10:2019-2034.

Harlow E & Lane D (1988) Antibodies: A Laboratory Manual. Cold Spring Harbor Laboratory Press, Cold Spring Harbor, N.Y.

Hauck W & Stanners C P (1995) Transcriptional Regulation of the Carcinoembryonic Antigen Gene. Identification of Regulatory Elements and Multiple Nuclear Factors. J Biol Chem 270:3602-3610.

Haynes R L, Zheng T & Nicchitta C V (1997) Structure and Folding of Nascent Polypeptide Chains During Protein Translocation in the Endoplasmic Reticulum. J Biol Chem 272:17126-17133.

Henikoff J G, Pietrokovski S, McCallum C M & Henikoff S (2000) Blocks-Based Methods for Detecting Protein Homology. Electrophoresis 21:1700-1706.

Henikoff S & Henikoff J G (1992) Amino Acid Substitution Matrices from Protein Blocks. Proc Natl Acad Sci USA 89:10915-10919.

Henikoff S & Henikoff J G (2000) Amino Acid Substitution Matrices. Adv Protein Chem 54:73-97.

Henttu P & Vihko P (1989) cDNA Coding for the Entire Human Prostate Specific Antigen Shows High Homologies to the Human Tissue Kallikrein Genes. Biochem Biophys Res Commun 160:903-910.

Hironaka S, Hasebe T, Kamijo T, Ohtsu A, Boku N, Yoshida S, Saitoh H & Ochiai A (2002) Biopsy Specimen Microvessel Density Is a Useful Prognostic Marker in Patients with T(2-4)M(0) Esophageal Cancer Treated with Chemoradiotherapy. Clin Cancer Res 8:124-130.

Huang C C, Novak W R, Babbitt P C, Jewett A I, Ferrin T E & Klein T E (2000) Integrated Tools for Structural and Sequence Alignment and Analysis. Pac Symp Biocomput230-241.

Hurley J H & Misra S (2000) Signaling and Subcellular Targeting by Membrane-Binding Domains. Annu Rev Biophys Biomol Struct 29:49-79.

Inaba K, Inaba M, Romani N, Aya H, Deguchi M, Ikehara S, Muramatsu S & Steinman R M (1992) Generation of Large Numbers of Dendritic Cells from Mouse Bone Marrow Cultures Supplemented with Granulocyte/Macrophage Colony-Stimulating Factor. J Exp Med 176:1693-1702.

Israeli R S, Powell C T, Fair W R & Heston W D (1993) Molecular Cloning of a Complementary DNA Encoding a Prostate-Specific Membrane Antigen. Cancer Res 53:227-230.

Ito T, Qiu H, Collins J A, Brill A B, Johnson D K & Griffin T W (1991) Preclinical Assessments of 90Y-Labeled C110 Anti-Carcinoembryonic Antigen Immunotoxin: A Therapeutic Immunoconjugate for Human Colon Cancer. Cancer Res 51:255-260.

Janetzki S, Palla D, Rosenhauer V, Lochs H, Lewis J J & Srivastava P K (2000) Immunization of Cancer Patients with Autologous Cancer-Derived Heat Shock Protein Gp96 Preparations: A Pilot Study. Int J Cancer 88:232-238.

Janeway C A, Jr. (1989) Approaching the Asymptote? Evolution and Revolution in Immunology. Cold Spring Harb Symp Quant Biol 54:1-13.

Janoff A S (1999) Liposomes: Rational Design. M. Dekker, New York.

Johnson J E, Giorgione J & Newton A C (2000) The C1 and C2 Domains of Protein Kinase C Are Independent Membrane Targeting Modules, with Specificity for Phosphatidylserine Conferred by the C1 Domain. Biochemistry 39:11360-11369.

Joki T, Nakamura M & Ohno T (1995) Activation of the Radiosensitive Egr-1 Promoter Induces Expression of the Herpes Simplex Virus Thymidine Kinase Gene and Sensitivity of Human Glioma Cells to Ganciclovir. Hum Gene Ther 6:1507-1513.

Karlin S & Altschul SF (1993) Applications and Statistics for Multiple High-Scoring Segments in Molecular Sequences. Proc Natl Acad Sci USA 90:5873-5877.

Kirpotin D, Park J W, Hong K, Zalipsky S, Li W L, Carter P, Benz C C & Papahadjopoulos D (1997) Sterically Stabilized Anti-Her2 Immunoliposomes: Design and Targeting to Human Breast Cancer Cells in Vitro. Biochemistry 36:66-75.

Kol A, Bourcier T, Lichtman A H & Libby P (1999) Chlamydial and Human Heat Shock Protein 60s Activate Human Vascular Endothelium, Smooth Muscle Cells, and Macrophages. J Clin Invest 103:571-577.

Kol A, Lichtman A H, Finberg R W, Libby P & Kurt-Jones E A (2000) Cutting Edge: Heat Shock Protein (Hsp) 60 Activates the Innate Immune Response: CD14 Is an Essential Receptor for Hsp60 Activation of Mononuclear Cells. J Immunol 164:13-17.

Kumar V, Cotran RS & Robbins S L (1997) Basic Pathology, 6th ed. W.B. Saunders Co., Philadelphia.

Kyte J & Doolittle R F (1982) A Simple Method for Displaying the Hydropathic Character of a Protein. J Mol Biol 157:105-132.

Labat-Moleur F, Steffan A M, Brisson C, Perron H, Feugeas O, Furstenberger P, Oberling F, Brambilla E & Behr J P (1996) An Electron Microscopy Study into the Mechanism of Gene Transfer with Lipopolyamines. Gene Ther 3:1010-1017.

Lammert E, Stevanovic S, Brunner J, Rammensee H G & Schild H (1997) Protein Disulfide Isomerase Is the Dominant Acceptor for Peptides Translocated into the Endoplasmic Reticulum. Eur J Immunol 27:1685-1690.

Lasic D D & Martin F J (1995) Stealth® Liposomes. CRC Press, Boca Raton, Fla., United States of America.

Lee H H, Morse SA & Olsvik Ø (1997) Nucleic Acid Amplification Technologies: Application to Disease Diagnosis. Birkhauser Boston, Cambridge, Mass., United States of America.

Lemmon M A & Ferguson K M (2000) Signal-Dependent Membrane Targeting by Pleckstrin Homology (PH) Domains. Biochem J 350 Pt 1:1-18.

Li Z & Srivastava PK (1993) Tumor Rejection Antigen Gp96/GRP94 Is an ATPase: Implications for Protein Folding and Antigen Presentation. EMBO J 12:3143-3151.

Linderoth N A, Popowicz A & Sastry S (2000) Identification of the Peptide-Binding Site in the Heat Shock Chaperone/Tumor Rejection Antigen Gp96 (GRP94). J Biol Chem 275:5472-5477.

Lohr F, Lo D Y, Zaharoff D A, Hu K, Zhang X, Li Y, Zhao Y, Dewhirst MW, Yuan F & Li C Y (2001) Effective Tumor Therapy with Plasmid-Encoded Cytokines Combined with in Vivo Electroporation. Cancer Res 61:3281-3284.

Mai K T, Isotalo P A, Green J, Perkins D G, Morash C & Collins J P (2000) Incidental Prostatic Adenocarcinomas and Putative Premalignant Lesions in Turp Specimens Collected before and after the Introduction of Prostrate-Specific Antigen Screening. Arch Pathol Lab Med 124:1454-1456.

Manome Y, Abe M, Hagen M F, Fine H A & Kufe D W (1994) Enhancer Sequences of the Df3 Gene Regulate Expression of the Herpes Simplex Virus Thymidine Kinase Gene and Confer Sensitivity of Human Breast Cancer Cells to Ganciclovir. Cancer Res 54:5408-5413.

Marin M, Noel D & Piechaczyk M (1997) Towards Efficient Cell Targeting by Recombinant Retroviruses. Mol Med Today 3:396-403.

Martoglio B & Dobberstein B (1998) Signal Sequences: More Than Just Greasy Peptides. Trends Cell Biol 8:410-415.

Maruyama-Tabata H, Harada Y, Matsumura T, Satoh E, Cui F, Iwai M, Kita M, Hibi S, Imanishi J, Sawada T & Mazda O (2000) Effective Suicide Gene Therapy in Vivo by EBV-Based Plasmid Vector Coupled with Polyamidoamine Dendrimer. Gene Ther7:53-60.

Medzhitov R & Janeway C A, Jr. (1997) Innate Immunity: The Virtues of a Nonclonal System of Recognition. Cell 91:295-298.

Multhoff G, Botzler C, Jennen L, Schmidt J, Ellwart J & Issels R (1997) Heat Shock Protein 72 on Tumor Cells: A Recognition Structure for Natural Killer Cells. J Immunol 158:4341-4350.

Munro S & Pelham HR (1987) A C-Terminal Signal Prevents Secretion of Luminal Er Proteins. Cell 48:899-907.

Natali P G, Roberts J T, Difilippo F, Bigotti A, Dent P B, Ferrone S & Liao S K (1987) Immunohistochemical Detection of Antigen in Human Primary and Metastatic Melanomas by the Monoclonal Antibody 140.240 and Its Possible Prognostic Significance. Cancer 59:55-63.

Needleman S B & Wunsch C D (1970) A General Method Applicable to the Search for Similarities in the Amino Acid Sequence of Two Proteins. J Mol Biol 48:443-453.

Neri D, Carnemolla B, Nissim A, Leprini A, Querze G, Balza E, Pini A, Tarli L, Halin C, Neri P, Zardi L & Winter G (1997) Targeting by Affinity-Matured Recombinant Antibody Fragments of an Angiogenesis Associated Fibronectin Isoform. Nat Biotechnol 15:1271-1275.

Nieland T J, Tan M C, Monne-van Muijen M, Koning F, Kruisbeek A M & van Bleek G M (1996) Isolation of an Immunodominant Viral Peptide That Is Endogenously Bound to the Stress Protein Gp96/GRP94. Proc Natl Acad Sci USA 93:6135-6139.

Norrby E & Cold Spring Harbor Laboratory. (1994) Vaccines 94 Modern Approaches to New Vaccines Including Prevention of AIDS. Cold Spring Harbor Laboratory Press, Plainview, N.Y.

O'Hagan D T, MacKichan M L & Singh M (2001) Recent Developments in Adjuvants for Vaccines against Infectious Diseases. Biomol Eng 18:69-85. Ohashi K, Burkart V, Flohe S & Kolb H (2000) Cutting Edge: Heat Shock Protein 60 Is a Putative Endogenous Ligand of the Toll-Like Receptor-4 Complex. J Immunol 164:558-561.

Ohtsuka E, Matsuki S, Ikehara M, Takahashi Y & Matsubara K (1985) An Alternative Approach to Deoxyoligonucleotides as Hybridization Probes by Insertion of Deoxyinosine at Ambiguous Codon Positions. J Biol Chem 260:2605-2608.

Ohtsuka K & Hata M (2000) Molecular Chaperone Function of Mammalian Hsp70 and Hsp40—a Review. Int J Hyperthermia 16:231-245. Park J W, Hong K, Kirpotin D B, Meyer O, Papahadjopoulos D & Benz CC (1997) Anti-Her2 Immunoliposomes for Targeted Therapy of Human Tumors. Cancer Lett 118:153-160.

Pasqualini R & Ruoslahti E (1996) Organ Targeting in Vivo Using Phage Display Peptide Libraries. Nature 380:364-366.

Pasqualini R, Koivunen E & Ruoslahti E (1997) Alpha V Integrins as Receptors for Tumor Targeting by Circulating Ligands. Nat Biotechnol 15:542-546.

PCT International Publication No. WO 93/25521 PCT International Publication No. WO 98/10795 Pearson W R & Lipman D J (1988) Improved Tools for Biological Sequence Comparison. Proc Natl Acad Sci USA 85:2444-2448.

Perez M S & Walker L E (1989) Isolation and Characterization of a Cdna Encoding the KS1/4 Epithelial Carcinoma Marker. J Immunol 142:3662-3667.

Prodromou C, Roe S M, Piper P W & Pearl L H (1997a) A Molecular Clamp in the Crystal Structure of the N-Terminal Domain of the Yeast Hsp90 Chaperone. Nat Struct Biol 4:477-482.

Prodromou C, Roe S M, O'Brien R, Ladbury J E, Piper P W & Pearl L H (1997b) Identification and Structural Characterization of the ATP/ADP-Binding Site in the Hsp90 Molecular Chaperone. Cell 90:65-75.

Richards C A, Austin E A & Huber B E (1995) Transcriptional Regulatory Sequences of Carcinoembryonic Antigen: Identification and Use with Cytosine Deaminase for Tumor-Specific Gene Therapy. Hum Gene Ther 6:881-893.

Richardson M D & Warnock D W (1993) Fungal Infection: Diagnosis and Management. Blackwell Scientific Publications, Oxford/Boston, Mass., United States of America.

Robert J, Menoret A, Srivastava P K & Cohen N (2001) Immunological Properties of Heat Shock Proteins Are Phylogenetically Conserved. Adv Exp Med Biol 484:237-249.

Romagnani S (1992) Induction of Th1 and Th2 Responses: A Key Role for the ‘Natural’ Immune Response? Immunol Today 13:379-381.

Rosser M F & Nicchitta C V (2000) Ligand Interactions in the Adenosine Nucleotide-Binding Domain of the Hsp90 Chaperone, GRP94. I. Evidence for Allosteric Regulation of Ligand Binding. J Biol Chem 275:22798-22805.

Rossolini G M, Cresti S, Ingianni A, Cattani P, Riccio M L & Satta G (1994) Use of Deoxyinosine-Containing Primers Vs Degenerate Primers for Polymerase Chain Reaction Based on Ambiguous Sequence Information. Mol Cell Probes 8:91-98.

Rotzschke O, Falk K, Wallny H J, Faath S & Rammensee H G (1990a) Characterization of Naturally Occurring Minor Histocompatibility Peptides Including H-4 and H-Y. Science 249:283-287.

Rotzschke O, Falk K, Deres K, Schild H, Norda M, Metzger J, Jung G & Rammensee H G (1990b) Isolation and Analysis of Naturally Processed Viral Peptides as Recognized by Cytotoxic T Cells. Nature 348:252-254.

Saltzman W M & Fung L K (1997) Polymeric Implants for Cancer Chemotherapy. Adv Drug Deliv Rev 26:209-230.

Sambrook et al. e (1989) Molecular Cloning: A Laboratory Manual. Cold Spring Harbor Laboratory Press, Cold Spring Harbor, N.Y.

Santoro M G (2000) Heat Shock Factors and the Control of the Stress Response. Biochem Pharmacol 59:55-63.

Saqi M A, Wild D L & Hartshorn M J (1999) Protein Analyst—a Distributed Object Environment for Protein Sequence and Structure Analysis. Bioinformatics 15:521-522.

Schena M (2000) Microarray Biochip Technology. Eaton Publishing, Natick, Mass., United States of America.

Schild H, Arnold-Schild D, Lammert E & Rammensee H G (1999) Stress Proteins and Immunity Mediated by Cytotoxic T Lymphocytes. Curr Opin Immunol 11:109-113.

Silhavy T J, Berman M L, Enquist L W & Cold Spring Harbor Laboratory. (1984) Experiments with Gene Fusions. Cold Spring Harbor Laboratory, Cold Spring Harbor, N.Y.

Simonova M, Weissleder R, Sergeyev N, Vilissova N & Bogdanov A, Jr. (1999) Targeting of Green Fluorescent Protein Expression to the Cell Surface. Biochem Biophys Res Commun 262:638-642.

Singh-Jasuja H, Scherer H U, Hilf N, Arnold-Schild D, Rammensee H G, Toes R E & Schild H (2000a) The Heat Shock Protein Gp96 Induces Maturation of Dendritic Cells and Down-Regulation of Its Receptor. Eur J Immunol 30:2211-2215.

Singh-Jasuja H, Toes R E, Spee P, Munz C, Hilf N, Schoenberger S P, Ricciardi-Castagnoli P, Neefjes J, Rammensee H G, Arnold-Schild D & Schild H (2000b) Cross-Presentation of Glycoprotein 96-Associated Antigens on Major Histocompatibility Complex Class I Molecules Requires Receptor-Mediated Endocytosis. J Exp Med 191:1965-1974.

Smith T F & Waterman M (1981) Comparison of Biosequences. Adv Appl Math 2:482-489.

Spee P & Neefjes J (1997) Tap-Translocated Peptides Specifically Bind Proteins in the Endoplasmic Reticulum, Including Gp96, Protein Disulfide Isomerase and Calreticulin. Eur J Immunol 27:2441-2449.

Starnes S L, Duncan B W, Kneebone J M, Fraga C H, States S, Rosenthal G L & Lupinetti F M (2000) Pulmonary Microvessel Density Is a Marker of Angiogenesis in Children after Cavopulmonary Anastomosis. J Thorac Cardiovasc Surg 120:902-907.

Stebbins C E, Russo A A, Schneider C, Rosen N, Hartl F U & Pavletich NP (1997) Crystal Structure of an Hsp90-Geldanamycin Complex: Targeting of a Protein Chaperone by an Antitumor Agent. Cell 89:239-250.

Storch G A (2000) Essentials of Diagnostic Virology. Churchill Livingstone, New York.

Suto R & Srivastava P K (1995) A Mechanism for the Specific Immunogenicity of Heat Shock Protein-Chaperoned Peptides. Science 269:1585-1588.

Tailor P G, Govindan M V & Patel P C (1990) Nucleotide Sequence of Human Prostatic Acid Phosphatase Determined from a Full-Length cDNA Clone. Nucleic Acids Res 18:4928.

Tam P, Monck M, Lee D, Ludkovski P, Leng EC, Clow K, Stark H, Scherrer P, Graham R W & Cullis P R (2000) Stabilized Plasmid-Lipid Particles for Systemic Gene Therapy. Gene Ther7:1867-1874.

Tamura Y, Peng P, Liu K, Daou M & Srivastava P K (1997) Immunotherapy of Tumors with Autologous Tumor-Derived Heat Shock Protein Preparations. Science 278:117-120.

Tijssen (1993) Laboratory Techniques in Biochemistry and Molecular Bioloqy-Hybridization with Nucleic Acid Probes. Elsevier, N.Y.

Todryk S, Melcher A A, Hardwick N, Linardakis E, Bateman A, Colombo M P, Stoppacciaro A & Vile R G (1999) Heat Shock Protein 70 Induced During Tumor Cell Killing Induces Th1 Cytokines and Targets Immature Dendritic Cell Precursors to Enhance Antigen Uptake. J Immunol 163:1398-1408.

Udono H & Srivastava P K (1993) Heat Shock Protein 70-Associated Peptides Elicit Specific Cancer Immunity. J Exp Med 178:1391-1396. Vijayasaradhi S, Bouchard B & Houghton A N (1990) The Melanoma Antigen GP75 Is the Human Homologue of the Mouse B (Brown) Locus Gene Product. J Exp Med 171:1375-1380.

U.S. Pat. No. 4,554,101

U.S. Pat. No. 4,554,101

U.S. Pat. No. 5,834,228

U.S. Pat. No. 5,872,011

U.S. Pat. No. 6,127,339

U.S. Pat. No. 5,574,172

U.S. Pat. No. 6,106,866

U.S. Pat. No. 5,994,392

U.S. Pat. No. 5,651,991

U.S. Pat. No. 5,786,387

U.S. Pat. No. 5,922,356

U.S. Pat. No. 5,688,931

U.S. Pat. No. 5,858,410

U.S. Pat. No. 4,551,482

U.S. Pat. No. 5,714,166

U.S. Pat. No. 5,510,103

U.S. Pat. No. 5,490,840

U.S. Pat. No. 5,855,900

U.S. Pat. No. 5,922,545)

U.S. Pat. No. 6,335,035

U.S. Pat. No. 6,312,713

U.S. Pat. No. 6,296,842

U.S. Pat. No. 6,287,587

U.S. Pat. No. 6,267,981

U.S. Pat. No. 6,262,127

U.S. Pat. No. 6,221,958

U.S. Pat. No. 6,120,787

U.S. Pat. No. 6,090,925

U.S. Pat. No. 6,245,740

U.S. Pat. No. 6,238,705

U.S. Pat. No. 6,238,704

U.S. Pat. No. 6,190,700

U.S. Pat. No. 4,235,871

U.S. Pat. No. 4,551,482

U.S. Pat. No. 6,197,333

U.S. Pat. No. 6,132,766

U.S. Pat. No. 6,200,598

U.S. Pat. No. 5,011,634

U.S. Pat. No. 6,056,938

U.S. Pat. No. 6,217,886

U.S. Pat. No. 5,948,767

U.S. Pat. No. 6,210,707

U.S. Pat. No. 6,180,084

U.S. Pat. No. 6,296,832

U.S. Pat. No. 5,111,867

U.S. Pat. No. 5,632,991

U.S. Pat. No. 5,849,877

U.S. Pat. No. 5,948,647

U.S. Pat. No. 6,054,561

U.S. Pat. No. 6,071,890

U.S. Pat. No. 5,326,902

U.S. Pat. No. 5,234,933

von Heijne G (1990) The Signal Peptide. J Membr Biol 115:195-201.

von Heijne G (1998) Life and Death of a Signal Peptide. Nature 396:111,113.

Walter P & Blobel G (1983) Preparation of Microsomal Membranes for Cotranslational Protein Translocation. Methods Enzymol 96:84-93.

Wang T F, Chang J H & Wang C (1993) Identification of the Peptide Binding Domain of HSC70. 18-Kilodalton Fragment Located Immediately after ATPase Domain Is Sufficient for High Affinity Binding. J Biol Chem 268:26049-26051.

Wassenberg J J, Dezfulian C & Nicchitta C V (1999) Receptor Mediated and Fluid Phase Pathways for Internalization of the ER Hsp90 Chaperone GRP94 in Murine Macrophages. J Cell Sci 112:2167-2175.

Wearsch P A & Nicchitta C V (1996) Purification and Partial Molecular Characterization of Grp94, an ER Resident Chaperone. Protein Expr Purif 7:114-121.

Wearsch P A & Nicchitta C V (1997) Interaction of Endoplasmic Reticulum Chaperone GRP94 with Peptide Substrates Is Adenine Nucleotide-Independent. J Biol Chem 272:5152-5156.

Weichselbaum R R, Hallahan D, Fuks Z & Kufe D (1994) Radiation Induction of Immediate Early Genes: Effectors of the Radiation-Stress Response. Int J Radiat Oncol Biol Phys 30:229-234.

White D O & Fenner F (1994) Medical Virology, 4th ed. Academic Press, San Diego, Calif., United States of America.

Whitley D, Goldberg S P & Jordan W D (1999) Heat Shock Proteins: A Review of the Molecular Chaperones. J Vasc Surg 29:748-751.

Yamazaki K, Nguyen T & Podack ER (1999) Cutting Edge: Tumor Secreted Heat Shock-Fusion Protein Elicits CD8 Cells for Rejection. J Immunol 163:5178-5182.

Yip HC, Karulin AY, Tary-Lehmann M, Hesse M D, Radeke H, Heeger P S, Trezza R P, Heinzel F P, Forsthuber T & Lehmann P V (1999) Adjuvant-Guided Type-1 and Type-2 Immunity: Infectious/Noninfectious Dichotomy Defines the Class of Response. J Immunol 162:3942-3949.

Yu O & Lian L J (1991) [Ca125 and Radioimmunoimaging in Monitoring of Epithelial Ovarian Carcinoma]. Zhonghua Fu Chan Ke Za Zhi 26:235-238, 252.

Zheng H, Dai J, Stoilova D & Li Z (2001) Cell Surface Targeting of Heat Shock Protein Gp96 Induces Dendritic Cell Maturation and Antitumor Immunity. J Immunol 167:6731-6735.

Zhu X, Zhao X, Burkholder WF, Gragerov A, Ogata CM, Gottesman ME & Hendrickson W A (1996) Structural Analysis of Substrate Binding by the Molecular Chaperone Dnak. Science 272:1606-1614.

It will be understood that various details of the invention can be changed without departing from the scope of the invention. Furthermore, the foregoing description is for the purpose of illustration only, and not for the purpose of limitation—the invention being defined by the claims.

What is claimed is:

1. An isolated polypeptide comprising a recombinant stress response polypeptide free of an antigen binding domain, wherein the recombinant stress response polypeptide comprises an extracellularly transported polypeptide when expressed in a host cell.

2. The polypeptide of claim 1, wherein the recombinant stress response polypeptide comprises a recombinant polypeptide selected from the group consisting of a Hsp60 polypeptide, a Hsp70 polypeptide, a Hsp90 polypeptide, and a calreticulin polypeptide.

3. The polypeptide of claim 2, wherein the Hsp90 polypeptide comprises a GRP94 polypeptide or a HSP90 polypeptide.

4. The polypeptide of claim 3, wherein the GRP94 polypeptide comprises:
(a) a polypeptide comprising an amino acid sequence of SEQ ID NO:2;
(b) a polypeptide substantially identical to SEQ ID NO:2;
(c) a polypeptide encoded by a nucleic acid of SEQ ID NO:1; or
(d) a polypeptide peptide encoded by a nucleic acid substantially identical to SEQ ID NO:1.

5. The polypeptide of claim 3, wherein the GRP94 polypeptide comprises a polypeptide encoded by a nucleic acid molecule comprising:
(a) an isolated nucleic acid molecule that hybridizes to a nucleic acid comprising a nucleotide sequence of SEQ ID NO:1 under wash stringency conditions represented by a wash solution having less than about 200 mM salt concentration and a wash temperature of greater than about 45° C., and that encodes a GRP94 polypeptide free of an antigen binding domain; and
(b) an isolated nucleic acid differing by at least one functionally equivalent codon from the isolated nucleic acid molecule of (a) above in nucleic acid sequence due to the degeneracy of the genetic code, and that encodes a GRP94 polypeptide encoded by the isolated nucleic acid of (a) above.

6. A composition for eliciting an immune response in a subject, the composition comprising:
(a) an immunostimulatory amount of a recombinant stress response polypeptide free of an antigen binding domain; and
(b) a pharmaceutically acceptable carrier.

7. The composition of claim 6, wherein the immunostimulatory amount comprises an amount sufficient to elicit an innate immune response.

8. The composition of claim 7, wherein the innate immune response comprises dendritic cell maturation.

9. The composition of claim 6, wherein the immunostimulatory amount comprises an amount sufficient to elicit an adaptive immune response.

10. The method of claim 9, wherein the adaptive immune response comprises an anti-tumor response.

11. The composition of claim 6, wherein the recombinant stress response polypeptide comprises a recombinant polypeptide selected from the group consisting of a a Hsp60, a Hsp70 polypeptide, a Hsp90 polypeptide, and a calreticulin polypeptide.

12. The composition of claim 11, wherein the Hsp90 polypeptide comprises a GRP94 polypeptide or a HSP90 polypeptide.

13. The composition of claim 12, wherein the GRP94 polypeptide comprises:
(a) a polypeptide comprising an amino acid sequence of SEQ ID NO:2;
(b) a polypeptide substantially identical to SEQ ID NO:2;
(c) a polypeptide encoded by a nucleic acid of SEQ ID NO:1; or
(d) a polypeptide peptide encoded by a nucleic acid substantially identical to SEQ ID NO:1.

14. The composition of claim 12, wherein the GRP94 polypeptide comprises a polypeptide encoded by a nucleic acid molecule comprising:
(a) an isolated nucleic acid molecule that hybridizes to a nucleic acid comprising a nucleotide sequence of SEQ ID NO:1 under wash stringency conditions represented by a wash solution having less than about 200 mM salt concentration and a wash temperature of greater than about 45° C., and that encodes a GRP94 polypeptide free of an antigenic peptide binding domain; and
(b) an isolated nucleic acid differing by at least one functionally equivalent codon from the isolated nucleic acid molecule of (a) above in nucleic acid sequence due to the degeneracy of the genetic code, and that encodes a GRP94 polypeptide encoded by the isolated nucleic acid of (a) above.

15. A method for eliciting an immune response in a subject, the method comprising administering to a subject a recombinant stress response polypeptide free of an antigenic peptide binding site, whereby an immune response in the subject is elicited.

16. The method of claim 15, wherein the subject comprises a mammal.

17. The method of claim 16, wherein the mammal comprises a human.

18. The method of claim 15, wherein the recombinant stress response polypeptide comprises an extracellularly transported polypeptide when expressed in a host cell.

19. The method of claim 15, wherein the recombinant stress response polypeptide comprises a recombinant polypeptide selected from the group consisting of a Hsp60 polypeptide, a Hsp70 polypeptide, a Hsp90 polypeptide, and a calreticulin polypeptide.

20. The method of claim 19, wherein the Hsp90 polypeptide comprises a GRP94 polypeptide or a HSP90 polypeptide.

21. The method of claim 15, wherein the GRP94 polypeptide comprises:
(a) a polypeptide comprising an amino acid sequence of SEQ ID NO:2;
(b) a polypeptide substantially identical to SEQ ID NO:2;
(c) a polypeptide encoded by a nucleic acid of SEQ ID NO:1; or
(d) a polypeptide encoded by a nucleic acid substantially identical to SEQ ID NO:1.

22. The method of claim 15, wherein the GRP94 polypeptide comprises a polypeptide encoded by a nucleic acid molecule comprising:
(a) an isolated nucleic acid molecule that hybridizes to a nucleic acid comprising a nucleotide sequence of SEQ ID NO:1 under wash stringency conditions represented by a wash solution having less than about 200 mM salt concentration and a wash temperature of greater than about 45° C., and that encodes a GRP94 polypeptide free of an antigenic peptide binding domain; and
(b) an isolated nucleic acid differing by at least one functionally equivalent codon from the isolated nucleic acid molecule of (a) above in nucleic acid sequence due to the degeneracy of the genetic code, and that encodes a GRP94 polypeptide encoded by the isolated nucleic acid of (a) above.

23. The method of claim 15, wherein the immune response comprises an innate immune response.

24. The method of claim 23, wherein the innate immune response comprises dendritic cell maturation.

25. The method of claim 15, wherein the immune response comprises an adaptive immune response.

26. The method of claim 25, wherein the adaptive immune response comprises an anti-tumor response.

27. The method of claim 25, wherein the adaptive immune response comprises an anti-infection response.

28. A method for inhibiting tumor growth in a subject, the method comprising administering to a subject a recombinant stress response polypeptide free of an antigen binding site, whereby tumor growth in a subject is inhibited.

29. The method of claim 28, wherein the subject comprises a mammal.

30. The method of claim 29, wherein the mammal comprises a human.

31. The method of claim 28, wherein the recombinant stress response polypeptide comprises an extracellularly transported polypeptide when expressed in a host cell.

32. The method of claim 28, wherein the recombinant stress response polypeptide comprises a recombinant polypeptide selected from the group consisting of a Hsp60 polypeptide, a Hsp70 polypeptide, a Hsp90 polypeptide, and a calreticulin polypeptide.

33. The method of claim 32, wherein the Hsp90 polypeptide comprises a GRP94 polypeptide or a HSP90 polypeptide.

34. The method of claim 33, wherein the GRP94 polypeptide comprises:
(a) a polypeptide comprising an amino acid sequence of SEQ ID NO:2;
(b) a polypeptide substantially identical to SEQ ID NO:2;
(c) a polypeptide encoded by a nucleic acid of SEQ ID NO:1; or
(d) a polypeptide encoded by a nucleic acie substantially identical to SEQ ID NO:1.

35. The method of claim 33, wherein the GRP94 polypeptide comprises a polypeptide encoded by a nucleic acid molecule comprising:
(a) an isolated nucleic acid molecule that hybridizes to a nucleic acid comprising a nucleotide sequence of SEQ ID NO:1 under wash stringency conditions represented by a wash solution having less than about 200 mM salt concentration and a wash temperature of greater than about 45° C., and that encodes a GRP94 polypeptide free of an antigenic peptide binding domain; and
(b) an isolated nucleic acid differing by at least one functionally equivalent codon from the isolated nucleic acid molecule of (a) above in nucleic acid sequence due to the degeneracy of the genetic code, and that encodes a GRP94 polypeptide encoded by the isolated nucleic acid of (a) above.

36. A method for inhibiting tumor metastasis in a subject, the method comprising administering to a subject a recombinant stress response polypeptide free of an antigen binding site, whereby tumor metastasis is inhibited.

37. The method of claim 36, wherein the subject comprises a mammal.

38. The method of claim 36, wherein the mammal comprises a human.

39. The method of claim 36, wherein the recombinant stress response polypeptide comprises an extracellularly transported polypeptide when expressed in a host cell.

40. The method of claim 36, wherein the recombinant stress response polypeptide comprises a recombinant polypeptide selected from the group consisting of a a Hsp60 polypeptide, a Hsp70 polypeptide, a Hsp90 polypeptide, and a calreticulin polypeptide.

41. The method of claim 40, wherein the Hsp90 polypeptide comprises a GRP94 polypeptide or a HSP90 polypeptide.

42. The method of claim 41, wherein the GRP94 polypeptide comprises:
(a) a polypeptide comprising an amino acid sequence of SEQ ID NO:2;
(b) a polypeptide substantially identical to SEQ ID NO:2;
(c) a polypeptide encoded by a nucleic acid of SEQ ID NO:1; or
(d) a polypeptide encoded by a nucleic acid substantially identical to SEQ ID NO:1.

43. The method of claim 41, wherein the GRP94 polypeptide comprises a polypeptide encoded by a nucleic acid molecule comprising:
(a) an isolated nucleic acid molecule that hybridizes to a nucleic acid comprising a nucleotide sequence of SEQ ID NO:1 under wash stringency conditions represented by a wash solution having less than about 200 mM salt concentration and a wash temperature of greater than about 45° C., and that encodes a GRP94 polypeptide free of an antigenic peptide binding domain; and
(b) an isolated nucleic acid differing by at least one functionally equivalent codon from the isolated nucleic acid molecule of (a) above in nucleic acid sequence due to the degeneracy of the genetic code, and that encodes a GRP94 polypeptide encoded by the isolated nucleic acid of (a) above.

44. A method of inhibiting tumor growth in a subject, the method comprising:
(a) transfecting a culture of healthy cells with a construct encoding a stress response polypeptide, wherein the stress response polypeptide comprises an extracellularly transported polypeptide when expressed in the healthy cell; and
(b) administering to a subject the culture of transfected healthy cells, whereby tumor growth in the subject is inhibited.

45. The method of claim 44, wherein the culture of healthy cells comprises a culture of non-cancerous cells.

46. The method of claim 44, wherein the culture of healthy cells comprises cells heterolgous to the subject.

47. The method of claim 44, wherein the stress response polypeptide comprises a secreted polypeptide.

48. The method of claim 44, wherein the recombinant stress response polypeptide comprises a recombinant polypeptide selected from the group consisting of a Hsp60 polypeptide, a Hsp70 polypeptide, a Hsp90 polypeptide, and a calreticulin polypeptide.

49. The method of claim 48, wherein the Hsp90 polypeptide comprises a GRP94 polypeptide or a HSP90 polypeptide.

50. The method of claim 49, wherein the GRP94 polypeptide comprises:
(a) a polypeptide comprising an amino acid sequence of SEQ ID NO:22;
(b) a polypeptide substantially identical to SEQ ID NO:22;
(c) a polypeptide encoded by a nucleic acid of SEQ ID NO:21; or
(d) a polypeptide peptide encoded by a nucleic acid substantially identical to SEQ ID NO:21.

51. The method of claim 49, wherein the GRP94 polypeptide comprises a polypeptide encoded by a nucleic acid molecule comprising:
(a) an isolated nucleic acid molecule that hybridizes to a nucleic acid comprising a nucleotide sequence of SEQ ID NO:21 under wash stringency conditions represented by a wash solution having less than about 200 mM salt concentration and a wash temperature of greater than about 45° C., and that encodes a GRP94 polypeptide free of an antigen binding domain; and
(b) an isolated nucleic acid differing by at least one functionally equivalent codon from the isolated nucleic acid molecule of (a) above in nucleic acid sequence due to the degeneracy of the genetic code, and that encodes a GRP94 polypeptide encoded by the isolated nucleic acid of (a) above.

52. The method of claim 44, wherein the subject comprises a mammal.

53. The method of claim 52, wherein the mammal comprises a human.

54. A method of inhibiting tumor metastasis in a subject, the method comprising:
(a) transfecting a culture of healthy cells with a construct encoding a stress response polypeptide, wherein the stress response polypeptide comprises an extracellularly transported polypeptide when expressed in the healthy cell; and
(b) administering to a subject the culture of transfected healthy cells, whereby tumor metastasis in the subject is inhibited.

55. The method of claim 54, wherein the culture of healthy cells comprises a culture of non-cancerous cells.

56. The method of claim 54, wherein the culture of healthy cells comprises cells heterolgous to the subject.

57. The method of claim 54, wherein the stress response polypeptide comprises a secreted polypeptide.

58. The method of claim 54, wherein the recombinant stress response polypeptide comprises a recombinant polypeptide selected from the group consisting of a Hsp60 polypeptide, a Hsp70 polypeptide, a Hsp90 polypeptide, and a calreticulin polypeptide.

59. The method of claim 58, wherein the Hsp90 polypeptide comprises a GRP94 polypeptide or a HSP90 polypeptide.

60. The method of claim 59, wherein the GRP94 polypeptide comprises:
(a) a polypeptide comprising an amino acid sequence of SEQ ID NO:22;
(b) a polypeptide substantially identical to SEQ ID NO:22;
(c) a polypeptide encoded by a nucleic acid of SEQ ID NO:21; or
(d) a polypeptide peptide encoded by a nucleic acid substantially identical to SEQ ID NO:21.

61. The method of claim 69, wherein the GRP94 polypeptide comprises a polypeptide encoded by a nucleic acid molecule comprising:
(a) an isolated nucleic acid molecule that hybridizes to a nucleic acid comprising a nucleotide sequence of SEQ ID NO:21 under wash stringency conditions represented by a wash solution having less than about 200 mM salt concentration and a wash temperature of greater than about 45° C., and that encodes a GRP94 polypeptide free of an antigen binding domain; and
(b) an isolated nucleic acid differing by at least one functionally equivalent codon from the isolated nucleic acid molecule of (a) above in nucleic acid sequence due to the degeneracy of the genetic code, and that encodes a GRP94 polypeptide encoded by the isolated nucleic acid of (a) above.

62. The method of claim 54, wherein the subject comprises a mammal.

63. The method of claim 62, wherein the mammal comprises a human.
